SEQUENCE LISTING (24 sequences)

SEQ ID NO: 1 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - DNA, 1302 bp, synthetic construct
  source 1..1302: mol_type = other DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaaccggttccggagccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgac

SEQ ID NO: 4 - protein, 431 aa, synthetic construct
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQTGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - DNA, 2559 bp, synthetic construct
  source 1..2559: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccaccggttccggagccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 6 - protein, 852 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGLASGGSMVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSATGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 7 - DNA, 1032 bp, synthetic construct
  source 1..1032: mol_type = other DNA
atggacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcccctggcggcggctga

SEQ ID NO: 8 - protein, 343 aa, synthetic construct
MDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGG

SEQ ID NO: 9 - DNA, 1614 bp, synthetic construct
  source 1..1614: mol_type = other DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaaccggttccggagccacgaacttctctctgttaaagcaagcaggagacgtggaagaaaaccccggtcctacgcgtatggacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcccctggcggcggctga

SEQ ID NO: 10 - protein, 537 aa, synthetic construct
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQTGSGATNFSLLKQAGDVEENPGPTRMDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGG

SEQ ID NO: 11 - DNA, 2877 bp, synthetic construct
  source 1..2877: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccaccggttccggagccacgaacttctctctgttaaagcaagcaggagacgtggaagaaaaccccggtcctacgcgtatggacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcccctggcggcggctga

SEQ ID NO: 12 - protein, 958 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGLASGGSMVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSATGSGATNFSLLKQAGDVEENPGPTRMDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGG

SEQ ID NO: 13 - DNA, 816 bp, synthetic construct
  source 1..816: mol_type = other DNA
atggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctag

SEQ ID NO: 14 - protein, 271 aa, synthetic construct
MDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

SEQ ID NO: 15 - DNA, 2655 bp, synthetic construct
  source 1..2655: mol_type = other DNA
atgtgccctcagaagctcactatctcctggtttgctattgtcctcctggtcagccccctcatggctatgtgggaactggaaaaagacgtgtacgtggtcgaggtggactggacacccgatgcccctggcgagacagtgaacctgacatgtgacacccccgaggaggacgatatcacctggacatccgatcagaggcacggagtgatcggatctggcaagaccctgacaatcaccgtgaaggagttcctggacgctggccagtacacatgccacaagggcggagagacactgtctcacagccacctgctgctgcacaagaaggagaacggaatctggtccacagagatcctgaagaacttcaagaacaagacctttctgaagtgtgaggcccccaactactctggccggttcacctgcagctggctggtgcagcgcaacatggacctgaagtttaacatcaagagctcctctagctcccctgatagcagggctgtgacatgtggcatggcctccctgtctgctgagaaggtgaccctggaccagagagattacgagaagtacagcgtgtcctgtcaggaggacgtgacatgcccaaccgccgaggagacactgccaatcgagctggccctggaggctcggcagcagaacaagtacgagaactactctaccagcttctttatccgcgacatcatcaagcctgatccccctaagaacctgcagatgaagccactgaagaactcccaggtggaggtgtcttgggagtaccccgactcctggtctacacctcactcttacttcagcctgaagttctttgtgcggatccagcgcaagaaggagaagatgaaggagacagaggagggatgtaaccagaagggcgcctttctggtggagaagacaagcaccgaggtgcagtgtaagggcggaaacgtgtgcgtgcaggcccaggatcggtactacaactctagctgctccaagtgggcttgcgtgccttgtagggtgagaagcggaggaggaggatccggaggaggaggatctggaggaggcctggcttccggaggatctatggtgagcgtgccaacagctagcccttccgcttcctctagctcctctcagtgtcggagctccatgtgccagtcccgctacctgctgttcctggccaccctggctctgctgaaccacctgagcctggctcgcgtgatcccagtgtccggaccagctaggtgtctgtctcagagccgcaacctgctgaagaccacagacgatatggtgaagacagcccgggagaagctgaagcactacagctgcaccgctgaggacatcgatcacgaggacatcacacgcgatcagacatctaccctgaagacctgtctgcctctggagctgcacaagaacgagagctgcctggccacaagggagacaagcagcaccacaagaggatcttgtctgccaccccagaagacaagcctgatgatgaccctgtgcctgggctccatctacgaggacctgaagatgtaccagaccgagtttcaggccatcaacgccgctctgcagaaccacaaccaccagcagatcatcctggacaagggcatgctggtggctatcgatgagctgatgcagtccctgaaccacaacggagagacactgaggcagaagcctccagtgggcgaggccgatccatacagagtgaagatgaagctgtgcatcctgctgcacgctttctctacaagggtggtgaccatcaacagagtgatgggatacctgtcctctgccacaggatccggcgctaccaacttttctctgctgaagcaggctggcgacgtggaggagaaccctggaccaatggatggacctaggctcctcctgctgctgctgctgggcgtgtccctgggaggagctaaggaggcttgcccaacaggactgtacacccactctggcgagtgctgtaaggcctgtaacctgggagagggagtggctcagccatgcggagctaaccagacagtgtgcgagccttgtctggactccgtgaccttctctgacgtggtgagcgccacagagccatgcaagccctgtaccgagtgcgtgggcctgcagtccatgtctgccccatgcgtggaggctgacgatgccgtgtgcagatgtgcttacggatactaccaggacgagacaacaggcaggtgtgaggcttgcagggtgtgcgaggctggaagcggcctggtgttttcctgccaggataagcagaacacagtgtgcgaggagtgtccagacggaacctacagcgatgaggccaaccacgtggacccttgcctgccatgtacagtgtgcgaggataccgagaggcagctgagagagtgtaccagatgggctgacgctgagtgcgaggagatcccaggacggtggatcacacgcagcaccccacctgagggaagcgactccacagctccttccacccaggagccagaggctccaccagagcaggatctgatcgcctctacagtggctggagtggtgaccacagtgatgggcagctcccagccagtggtgaccagaggcaccacagacaacctgatccccgtgtactgcagcatcctggccgctgtggtggtggggctggtggcatacattgctttcaagagatggaataggggaatcctgtga

SEQ ID NO: 16 - protein, 884 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGLASGGSMVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSATGSGATNFSLLKQAGDVEENPGPMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

SEQ ID NO: 17 - DNA, 3417 bp, synthetic construct
  source 1..3417: mol_type = other DNA
atgtgccctcagaaactgactatctcctggtttgctattgtcctcctcgtgtcccctctcatggctatgtgggaactggaaaaagacgtgtacgtggtcgaggtggactggaccccagatgctccaggagagacagtgaacctgacatgtgacacccctgaggaggacgatatcacctggacatctgatcagagacacggcgtgatcggaagcggcaagaccctgacaatcaccgtgaaggagttcctggacgccggacagtacacctgccacaagggcggagagacactgtctcacagccacctgctgctgcacaagaaggagaacggcatctggagcaccgagatcctgaagaacttcaagaacaagacatttctgaagtgtgaggcccccaactacagcggacggttcacctgctcctggctggtgcagcgcaacatggacctgaagtttaacatcaagagctcctctagctcccctgattccagggctgtgacctgtggcatggcctccctgtctgctgagaaggtgacactggaccagagagattacgagaagtacagcgtgtcctgtcaggaggacgtgacatgccctaccgccgaggagacactgccaatcgagctggccctggaggctcggcagcagaacaagtacgagaactactctacaagcttctttatccgcgacatcatcaagcccgatccccctaagaacctgcagatgaagcctctgaagaactctcaggtggaggtgagctgggagtacccagactcctggtctaccccccactcttacttcagcctgaagttctttgtgcggatccagcgcaagaaggagaagatgaaggagacagaggagggctgtaaccagaagggagcctttctggtggagaagacaagcaccgaggtgcagtgtaagggcggaaacgtgtgcgtgcaggcccaggataggtactacaactctagctgctccaagtgggcttgcgtgccatgtagggtgagatccggaggaggaggatctggaggaggaggaagcggaggaggcctggcttccggaggctctatggtgagcgtgccaaccgctagcccatccgcttcctctagctcctctcagtgtcggagctccatgtgccagagccgctacctgctgttcctggccacactggctctgctgaaccacctgtccctggctagagtgatccccgtgagcggaccagctaggtgtctgtctcagagccgcaacctgctgaagaccacagacgatatggtgaagaccgcccgggagaagctgaagcactactcctgcacagctgaggacatcgatcacgaggacatcacccgcgatcagacatccaccctgaagacatgtctgcccctggagctgcacaagaacgagtcttgcctggccacaagggagacaagcagcaccacaagaggcagctgtctgccaccccagaagacatccctgatgatgaccctgtgcctgggaagcatctacgaggacctgaagatgtaccagaccgagttccaggccatcaacgccgctctgcagaaccacaaccaccagcagatcatcctggacaagggcatgctggtggctatcgatgagctgatgcagagcctgaaccacaacggcgagacactgaggcagaagcctccagtgggagaggccgatccttacagagtgaagatgaagctgtgcatcctgctgcacgctttttccacaagggtggtgaccatcaacagagtgatgggctacctgtcctctgccacaggatctggcgctaccaacttcagcctgctgaagcaggctggcgacgtggaggagaacccaggacctatggattttcaggtgcagatcttctcctttctgctgatctccgcttctgtgatcatgtccaggggagaggtgcagctgctgcagtctggccctgagctggagaagccaggagcctccgtgatgatctcttgcaaggctagcggcagctccttcaccggatacaacatgaactgggtgaggcagaacatcggcaagagcctggagtggatcggagccatcgacccatactacggaggcacctcctacaaccagaagtttaagggcagagccacactgaccgtggacaagtctagctccaccgcttacatgcacctgaagtccctgacatctgaggatagcgccgtgtactactgcgtgagcggaatggagtactggggacagggcacatccgtgaccgtgtctagcggcggaggaggctctggcggcggcggctccggcggaggaggaagcgacgtggtcatgacacagaccccactgtccctgcccgtgagcctgggcgatcaggccagcatctcctgcaggtcctctcagtccctggtgcacagaaacggcaacacctacctgcactggtacctgcagaagccaggacagtctcccaagctgctgatccacaaggtgagcaaccggttctccggcgtgcctgaccgcttttctggaagcggctccggaacagatttcaccctgaagatcagcagggtggaggctgaggacctgggagtgtacttctgttctcagagcacccacgtgccccctctgacatttggcgctggaaccaagctggagctgacaggcctgctgggagtgtctctgggaggagctaaggaggcttgcccaacaggcctgtacacccacagcggagagtgctgtaaggcctgtaacctgggagagggagtggctcagccatgcggagctaaccagaccgtgtgcgagccctgtctggactctgtgacattcagcgatgtggtgtccgctaccgagccttgcaagccatgtacagagtgcgtgggcctgcagtccatgtctgccccatgcgtggaggctgacgatgccgtgtgcaggtgtgcttacggctactaccaggacgagacaacaggaaggtgtgaggcttgcagggtgtgcgaggctggatccggactggtgttttcttgccaggataagcagaacaccgtgtgcgaggagtgtccagacggcacatacagcgatgaggccaaccacgtggacccctgcctgccttgtaccgtgtgcgaggatacagagaggcagctgagagagtgtaccagatgggctgacgctgagtgcgaggagatcccaggccggtggatcacacgctctaccccaccagagggaagcgactccacagctcccagcacccaggagcctgaggctcctccagagcaggatctgatcgcctctaccgtggctggcgtggtgaccacagtgatgggaagctcccagcctgtggtgacacgcggcaccacagataacctgatcccagtgtactgctccatcctggccgctgtggtggtgggactcgtggcatacattgctttcaaacggtggaataggggaatcctgtga

SEQ ID NO: 18 - protein, 1138 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGLASGGSMVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSATGSGATNFSLLKQAGDVEENPGPMDFQVQIFSFLLISASVIMSRGEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELTGLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

SEQ ID NO: 19 - DNA, 1342 bp, synthetic construct
  source 1..1342: mol_type = other DNA
atggattttcaggtccagattttctccttcctcctcatttcagccagcgtcattatgtctcgggggacacctgactgttacttcagccacagtcccatctcctccaacttcaaagtgaagtttagagagttgactgaccacctgcttaaagattacccagtcactgtggccgtcaatcttcaggacgagaagcactgcaaggccttgtggagcctcttcctagcccagcgctggatagagcaactgaagactgtggcagggtctaagatgcaaacgcttctggaggacgtcaacaccgagatacattttgtcacctcatgtaccttccagcccctaccagaatgtctgcgattcgtccagaccaacatctcccacctcctgaaggacacctgcacacagctgcttgctctgaagccctgtatcgggaaggcctgccagaatttctctcggtgcctggaggtgcagtgccagccggactcctccaccctgctgcccccaaggagtcccatagccctagaagccacggagctcccagagcctcggcccaggcagaccggttccggagccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaag

SEQ ID NO: 20 - protein, 446 aa, synthetic construct
MDFQVQIFSFLLISASVIMSRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQTGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - DNA, 1317 bp, synthetic construct
  source 1..1317: mol_type = other DNA
atggattttcaggtccagatcttctcctttctgctgatttccgcctctgtgatcatgtccagaggcacccaggactgctctttccagcacagccctatcagctccgattttgccgtgaagatcagggagctgtctgactacctgctgcaggattatccagtgaccgtggccagcaatctgcaggacgaggagctgtgcggaggcctgtggaggctggtgctggcacagcggtggatggagagactgaagacagtggccggctctaagatgcagggcctgctggagcgggtgaacaccgagatccacttcgtgacaaagtgcgcctttcagccccctccaagctgtctgcggtttgtgcagaccaatatcagcagactgctgcaggagacatccgagcagctggtggccctgaagccatggatcaccaggcagaacttcagcaggtgcctggagctgcagtgtcagccagactctagcacactgccccctccatggtccccccgccctctggaggcaaccgcaccaacagccggctccggcgccaccaacttttctctgctgaagcaggccggcgatgtggaggagaatccaggccccatggtgagcaagggcgaggagctgttcacaggcgtggtgcccatcctggtggagctggacggcgatgtgaatggccacaagtttagcgtgtccggagagggagagggcgatgcaacctacggcaagctgacactgaagttcatctgcaccacaggcaagctgcctgtgccatggcccaccctggtgaccacactgacatacggcgtgcagtgtttctcccggtatcccgaccacatgaagcagcacgatttctttaagtctgccatgcctgagggctacgtgcaggagaggaccatcttctttaaggacgatggcaactataagacccgcgccgaggtgaagttcgagggcgacacactggtgaaccggatcgagctgaagggcatcgactttaaggaggatggcaatatcctgggccacaagctggagtacaactataattcccacaacgtgtatatcatggccgataagcagaagaacggcatcaaggtgaacttcaagatcagacacaatatcgaggacggctctgtgcagctggccgatcactaccagcagaacaccccaatcggcgacggacctgtgctgctgccagataatcactatctgtctacacagagcgccctgtccaaggaccctaacgagaagagggatcacatggtgctgctggagtttgtcactgccgctgggattaccctgggaatggatgagctgtataaataa

SEQ ID NO: 22 - protein, 438 aa, synthetic construct
MDFQVQIFSFLLISASVIMSRGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - DNA, 2502 bp, synthetic construct
  source 1..2502: mol_type = other DNA
atgtgccatcagcagctggtcatctcttggtttagtctggtgtttctggcttctccactggtcgcaatctgggaactgaaaaaggacgtgtacgtggtggagctggactggtatcctgatgccccaggcgagatggtggtgctgacctgtgacacacccgaggaggatggcatcacctggacactggatcagagctccgaggtgctgggaagcggcaagaccctgacaatccaggtgaaggagttcggcgacgcaggacagtacacctgccacaagggaggcgaggtgctgtctcacagcctgctgctgctgcacaagaaggaggacggcatctggtccacagacatcctgaaggatcagaaggagcccaagaacaagaccttcctgaggtgtgaggccaagaattattctggccgctttacctgctggtggctgaccacaatctccaccgatctgacattttctgtgaagtctagcaggggatcctctgaccctcagggagtgacatgcggagcagccaccctgagcgccgagcgggtgagaggcgataacaaggagtacgagtattctgtggagtgtcaggaggacagcgcctgcccagcagcagaggagtccctgcccatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccccgacacctggtccacacctcacagctatttctccctgaccttttgcgtgcaggtgcagggcaagtccaagagggagaagaaggaccgcgtgttcaccgataagacatctgccaccgtgatctgccggaagaacgcctccatctctgtgagggcccaggatcgctactattctagctcctggtctgagtgggccagcgtgccttgctccggcggcggcggctctggaggaggaggaagcggaggaggaggctccagaaatctgccagtggcaaccccagaccctggaatgttcccatgtctgcaccactcccagaacctgctgcgggccgtgtctaatatgctgcagaaggccagacagacactggagttttacccttgcaccagcgaggagatcgaccacgaggatatcacaaaggataagacctccacagtggaggcctgtctgccactggagctgaccaagaacgagtcttgcctgaatagcagggagacatccttcatcaccaacggctcttgtctggccagccgcaagacatcctttatgatggccctgtgcctgtctagcatctacgaggacctgaagatgtatcaggtggagttcaagaccatgaacgccaagctgctgatggacccaaagcggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttaggatccgcgccgtgacaatcgacagagtgatgtcttatctgaacgcaagcggatccggagcaaccaacttcagcctgctgaagcaggccggcgatgtggaggagaatccaggaccaatggacggaccaaggttattactgctgctgctgctgggcgtgtctctgggaggagcaaaggaggcatgcccaacaggcctgtacacccacagcggagagtgctgtaaggcatgtaacctgggagagggagtggcacagccttgcggagccaatcagacagtgtgcgagccatgtctggactctgtgaccttcagcgatgtggtgtccgccacagagccctgcaagccttgtaccgagtgcgtgggcctgcagtctatgagcgccccttgcgtggaggcagacgatgccgtgtgcagatgtgcctacggctactatcaggatgagacaacaggccggtgtgaggcatgccgggtgtgcgaggcaggatctggcctggtgtttagctgccaggacaagcagaacacagtgtgcgaggagtgtccagacggcacctattccgatgaggccaatcacgtggatccatgcctgccctgtacagtgtgcgaggacaccgagcggcagctgagagagtgtaccagatgggcagatgcagagtgcgaggagatcccaggccggtggatcacaagaagcaccccacccgagggctccgattctacagcaccttccacccaggagccagaggcacctccagagcaggacctgatcgccagcacagtggcaggagtggtgaccacagtgatgggaagctcccagccagtggtgaccaggggcaccacagacaacctgatccccgtgtactgctccatcctggcagcagtggtggtgggcctggtggcctatatcgctttcaagaggtggaatcggggcatcctgtaa

SEQ ID NO: 24 - protein, 833 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

(19) United States
(12) Patent Application Publication
(10) Pub. No.: US 2025/0325583 A1
(43) Pub. Date: Oct. 23, 2025

(54) ENGINEERED PROGENITOR CELLS AND METHODS OF USE

(71) Applicant: ECOLE POLYTECHNIQUE FEDERALE DE LAUSANNE, LAUSANNE (CH)

(72) Inventors: MICHELE DE PALMA, LAUSANNE (CH); ALI GHASEMI, RENENS VD (CH); AMAIA MARTINEZ-USATORRE, LAUSANNE (CH)

(21) Appl. No.: 18/870,729

(22) PCT Filed: May 30, 2023
(86) PCT No.: PCT/EP2023/064391

§ 371 Date: Dec. 2, 2024

(30) Foreign Application Priority Data
Jun. 1, 2022 (EP) 22176819.5

(51) Int. Cl. A61K 35/15; A61K 38/20; A61K 40/19; A61K 40/24; A61P 35/00; C12N 5/0784

(52) U.S. Cl. CPC A61K 35/15; A61K 38/208; A61K 40/19; A61K 40/24; A61P 35/00; C12N 5/0639; C12N 2501/145; C12N 2501/22; C12N 2501/2306; C12N 2501/2312; C12N 2501/26

(57) ABSTRACT

Disclosed herein are engineered progenitor cells and methods of using the same. Also disclosed herein are engineered cells differentiated from engineered progenitor cells of the present disclosure. Also disclosed herein are methods of treating a condition in a subject by administering an engineered progenitor cell or an engineered cell differentiated from an engineered progenitor cell of the present disclosure.

### CROSS-REFERENCED APPLICATION

This application claims the benefit of priority to European Patent Application No. EP22176819, filed on Jun. 1, 2022, the entire contents of each of which is incorporated herein by reference.

### INCORPORATION BY REFERENCE OF SEQUENCE LISTING

The instant application contains a Sequence Listing, which has been submitted via Patent Center. The Sequence Listing titled 212225.701601_PCT_SL.xml, which was created on May 17, 2023, and is 51,207 bytes in size, is hereby incorporated by reference in its entirety.

### FIELD

The present disclosure relates generally to engineered dendritic progenitor cells and methods of using the same.

### BACKGROUND

Immunotherapy can be employed for the treatment of various human diseases, such as infections, degenerative conditions, and cancer. In cancer, immunotherapy sometimes involves stimulating the patient's own immune system to attack cancer cells or other cellular components of the tumor.

Eliciting or enhancing cancer-specific T lymphocytes by vaccinating the patient against tumor-associated antigens (TAAs) represents an attractive means of treating a subject by immunotherapy. One type of cancer vaccine involves the use of dendritic cells (DCs). DCs are a family of immune cells endowed with the ability to capture and present TAAs to T lymphocytes through a variety of mechanisms, priming potent effector responses against the tumor. DCs are also capable of migration between lymphoid and non-lymphoid tissues and modulating cytokine and chemokine gradients to control inflammation and lymphocyte homing. However, improving the efficacy of DCs for therapeutic use has been challenging.

Immune cell engineering as described herein provides a means to improve the efficacy of DC vaccines. Autologous cell-based platform capable of producing and expanding cDC1 in vivo as described herein efficiently uptake and present tumor-associated antigens (TAAs) and induce strong and broad T-cell responses against multiple TAAs, making them attractive therapeutics against a broad range of cancers. Accordingly, the present disclosure provides methodology for the generation of a DC progenitor that efficiently generates cDC1 in vivo and that does not require antigen loading ex vivo, therefore providing the means for a tumor agnostic DC vaccine.

### SUMMARY

Disclosed herein are in vitro cell compositions that comprises a synthetically partially-differentiated dendritic cell progenitor, wherein the synthetically partially-differentiated dendritic cell progenitor has a phenotype of: CD115⁺, CD11c⁻, and Clec9A⁻ as determined by flow cytometry. In some embodiments, the phenotype of the synthetically partially differentiated dendritic progenitor cell further comprises one or more phenotypes selected from CD11b⁻, MHCII⁻, CD45R/B220⁻, and cKIT⁻ as determined by flow cytometry.

Also disclosed herein are differentiated cDC1 or cDC2 dendritic cells differentiated from a synthetically partially-differentiated dendritic cell progenitor described herein. In some embodiments, the synthetically-differentiated cDC1 or cDC2 is an engineered dendritic cell expressing an interleukin or an effector. In some embodiments, the engineered dendritic cell expresses the interleukin, wherein the interleukin is IL12. In some embodiments, the engineered dendritic cell expresses the effector, wherein the effector is selected from the group consisting of: extracellular vesicle-internalizing receptor (EVIR), FMS-like tyrosine kinase 3 ligand (FLT3L), IL-12, TNF-α, IL-1, IL-2, IL-6, CXCL8, interferon (IFN), GM-CSF, and G-CSF.

Also disclosed herein are in vitro cell compositions that comprises a synthetically partially-differentiated dendritic cell progenitor, wherein the synthetically partially-differentiated dendritic cell progenitor comprises one or more phenotypes selected from CD115⁺, CD34⁺, CD3⁻, CD19⁻, CD335⁻, CD66b⁻, CD10⁻, and CD14⁻ as determined by flow cytometry.

Also disclosed herein are antigen-presenting cells (APCs) differentiated from synthetically partially-differentiated dendritic cell progenitors described herein. In some embodiments, the APC is an engineered dendritic cell expressing an interleukin or an effector. In some embodiments, the engineered dendritic cell expresses the interleukin, wherein the interleukin is IL12. In some embodiments, the engineered dendritic cell expresses the effector, wherein the effector is selected from the group consisting of: extracellular vesicle-internalizing receptor (EVIR), FMS-like tyrosine kinase 3 ligand (FLT3L), GM-CSF, IL-6, IL-12, IFNα2β, IFNγ, SCF, and TNF-α.

Also disclosed herein are methods of making a synthetically partially differentiated dendritic cell progenitor, the method comprising: (a) obtaining a shortly-expanded hematopoietic stem/progenitor cell (HSPC), and (b) contacting the shortly-expanded HSPC with a synthetic medium comprising FMS-like tyrosine kinase 3 ligand (FLT3L) and GM-CSF, with or without IL-1, IL-2, IL-4, IL-6, IL-12, CXCL8, G-CSF, TNF-α, IFNa, PGE2, or retronectin, in an amount sufficient to differentiate the HSPC cell into a synthetically partially-differentiated dendritic cell progenitor having a phenotype of: CD115⁺, CD11c⁻, and Clec9A⁻, as determined by flow cytometry. In some embodiments, the method further comprises contacting the HSPC in a medium comprising: FBS, L-glutamine, SCF, TPO, FLT3L, IL-3, IL-6, and IL-1b, thereby making the shortly-expanded HSPC prior to the contacting of (b).

Also disclosed herein are methods of making a synthetically partially differentiated dendritic cell progenitor, the method comprising: (a) obtaining a shortly-expanded CD34⁺ human hematopoietic stem progenitor cell (human HSPC); and (b) contacting the shortly-expanded human HSPC with a synthetic medium comprising FMS-like tyrosine kinase 3 ligand (FLT3L), IL-3, IL-6, TPO, and SCF, with or without IFNγ, IL-12, retronectin, TNF-α, or UM729, in an amount sufficient to differentiate the HSPC cell into a synthetically partially-differentiated dendritic cell progenitor having one or more phenotypes selected from CD115⁺, CD34⁺, CD3⁻, CD19⁻, CD335⁻, CD66b⁻, CD10⁻, and CD14⁻ as determined by flow cytometry.

Also disclosed herein are pharmaceutical compositions for use in treatment of a condition, comprising: (a) an in vitro cell composition as described herein, and (b) a pharmaceutically-acceptable excipient, diluent, or carrier. Also disclosed herein are methods of treating a condition in a subject in need thereof, the method comprising administering to the subject a pharmaceutical composition that comprises: (a) an in vitro cell composition as described herein, and (b) a pharmaceutically-acceptable excipient, diluent, or carrier. In some embodiments, the condition is a cancer. In some embodiments, the pharmaceutical composition further comprises an interleukin or an effector. In some embodiments, the differentiated cDC1 or cDC2 dendritic cell is an engineered dendritic cell that expresses an interleukin or an effector. In some embodiments, the APC is an engineered dendritic cell that expresses an interleukin or an effector. In some embodiments, the interleukin is IL-12. In some embodiments, the effector is selected from the group consisting of: extracellular vesicle-internalizing receptor (EVIR), FMS-like tyrosine kinase 3 ligand (FLT3L), IL-12, TNF-α, IL-1, IL-2, IL-6, CXCL8, interferon (IFN), GM-CSF, and G-CSF. In some embodiments, the effector is not expressed on a cell of the cancer.

### BRIEF DESCRIPTION OF THE DRAWINGS

Novel features of exemplary embodiments are set forth with particularity in the appended claims. A better understanding of the features and advantages will be obtained by reference to the following detailed description that sets forth illustrative embodiments, in which the principles of the disclosed systems and methods are utilized, and the accompanying drawings of which:

FIGS. 1A-1C depict partial differentiation of host hematopoietic stem/progenitor cells into dendritic cell progenitors (DCPs) described herein. FIG. 1A outlines a protocol for preparation of the DCPs. FIG. 1B shows the purity of the isolated DCPs. FIG. 1C shows the phenotype of the isolated DCPs.

FIGS. 2A-2K depict the ability of various dendritic cells, including monocyte-derived dendritic cells (moDCs), conventional type I dendritic cells (cDC1), and DCPs of the present disclosure, to produce mature cDC1 in a tumor-free animal. FIG. 2A outlines a protocol for the adoptive transfer of the dendritic cells. FIGS. 2B-2D, and 2K show the phenotype of the moDC (FIGS. 2B and 2K), cDC1 (FIG. 2C) and DCPs of the present disclosure (FIG. 2D). FIGS. 2B and 2K each show FACS analysis of the same data for determining % of cells with moDC phenotype utilizing different inclusion criteria. FIGS. 2E-2F show the differentiation of the dendritic cells into splenic cDC1 (FIG. 2E) and cDC2 (FIG. 2F) after infusion. FIG. 2G shows the engraftment of donor-derived cells after infusion. FIGS. 2H-2J show the differentiation of donor-derived cells into various dendritic cell types in the spleen after infusion of moDC (FIG. 2H), cDC1 (FIG. 2I) or DCPs of the present disclosure (FIG. 2J).

FIGS. 3A-3I depict differentiation of DCPs of the present disclosure after systemic administration to a tumor-bearing animal. FIG. 3A shows a workflow of the administration. FIG. 3B shows the gating strategy for the identification of intratumoral donor-derived cDC1 and cDC2. FIG. 3C shows flow cytometry analysis of tumor-derived cells and splenocytes. FIGS. 3D-3E and 3H-3I show that donor-derived DCPs efficiently differentiate into cDCs. FIGS. 3D and 3H, each shows a pie chart derived from the same data showing relative proportion of differentiated cDCs and other type of cells, with the data in each figure processed with different inclusion criteria. Similarly, FIGS. 3E and 3I, each shows a pie chart derived from the same data showing relative proportion of differentiated cDCs and other type of cells, with the data in each figure processed with different inclusion criteria. FIGS. 3F-3G show the amount of donor-derived cDCs in the tumor (FIG. 3F) and spleen (FIG. 3G).

FIGS. 4A-4F depict identification of interleukins (ILs) that promote T cell activation by cDC1-like cells. FIG. 4A depicts a workflow of the experiment. FIG. 4B-4C show the effect of ILs on DCP differentiation into cDC1. FIGS. 4D-4F show that IL-12 enables cDC1-like cells to promote robust IFNγ production by both OT-I and OT-II T cells.

FIGS. 5A-5H show differentiation of DCPs expressing IL-12 or IL-2 in a tumor-free animal. FIG. 5A shows flow cytometry analysis of cDC1-like cells transduced with lentiviral vectors expressing either IL-12 or IL-2 together with GFP. FIGS. 5B and 5H shows that transduced cells secreted IL-12 and IL-2 by ELISA. FIG. 5H is an alternate representation of the same data shown in FIG. 5B, but with different curve fitting criteria. FIG. 5C shows a workflow of the administration of transduced DCPs. FIGS. 5D-5E illustrate flow cytometry analysis of splenocytes, showing transduced (GFP+) donor-derived cells. FIGS. 5F-5G show that transduced, donor-derived DCPs efficiently differentiate into cDCs.

FIGS. 6A-6C depict activation of antigen-specific T cells from DCPs expressing IL-12 and a tumor antigen (OVA). FIG. 6A is a schematic of the experiment. FIGS. 6B-6C show flow cytometry of splenocytes, indicating robust expansion of OVA-reactive T cells.

FIGS. 7A-7G show that DCPs expressing IL-12 and a tumor antigen block tumor initiation. FIG. 7A is a workflow of the experiment. FIGS. 7B-7C show flow cytometry of blood collected from mice 4 days after tumor challenge, indicating proportions of activated CD4⁺ and CD8⁺ T cells. FIG. 7D shows the amount of OVA-reactive T cells in the same blood samples. FIG. 7E depicts the change in tumor volume over time following tumor challenge. FIGS. 7F-7G show robust expansion of OVA-reactive effector T cells in the spleen.

FIGS. 8A-8F show that DCPs expressing IL-12 and a tumor antigen inhibit tumor growth. FIG. 8A is a workflow of the experiment. FIG. 8B shows that DCPs of the present disclosure inhibited tumor growth. FIGS. 8C-8F show the presence of OVA-reactive T cells in blood (FIG. 8C), spleen (FIG. 8D), tumor-draining lymph node (tdLN) (FIG. 8E) and tumor (FIG. 8F).

FIGS. 9A-9M depict vaccination using DCPs engineered to express IL-12 and EVIR. FIGS. 9A-9M show that DCPs expressing IL-12 and an EVIR inhibit tumor growth. FIG. 9A is a workflow of the experiment. FIG. 9B outlines tumor growth as a function of time after infusion of DCPs expressing IL-12 and EVIR. FIGS. 9C-9F show flow cytometry analysis of the tumor microenvironment. FIG. 9G shows a workflow of a mixed administration of melanoma cells. FIG. 9H outlines tumor growth as a function of time based after infusion of DCPs expressing IL-12 and EVIR. FIGS. 9I-9M depict flow cytometry of spleen and tdLN, showing the presence of OVA and non-OVA reactive T cells.

FIGS. 10A-10J depict vaccination with DCPs engineered to express IL-12 and FLT3L. FIG. 10A shows flow cytometry of transduced, cDC1-like cells. FIG. 10B shows that FLT3L was produced by the transduced cells, as determined by ELISA. FIG. 10J is an alternate representation of the same data shown in FIG. 10B, but with different curve fitting criteria. FIG. 10C is a workflow for the DCP infusion study. FIG. 10D outlines tumor growth as a function of time after DCP infusion. FIG. 10E shows the serum level of transgenic cytokines at different timepoints after the last DCP infusion FIGS. 10F-10G illustrates flow cytometry analysis of intra-tumoral T cells, showing robust expansion of CD8⁺ and CD4⁺ T cells. FIGS. 10H-10I show flow cytometry analysis of T cells in tdLNs.

FIGS. 11A-11L illustrates a comparison between DCPs of the present disclosure with moDCs and cDC1, each expressing IL-12 and FLT3L. FIG. 11A is a workflow of the experiment. FIG. 11B outlines tumor growth as a function of time after dendritic cell infusion. FIGS. 11C and 11D show flow cytometry analysis of tumors in treated mice. FIG. 11E shows flow cytometry analysis of tdLNs of treated mice. FIGS. 11F-11H illustrates analysis of tumor-derived T cells stimulated ex vivo, showing robust T cells activation. FIGS. 11I-11L show massive infiltration of T cells into the tumor microenvironment of mice that received DCPs engineered to express IL-12 and FLT3L.

FIGS. 12A-12H depict vaccination using DCPs of the present disclosure against another cancer type. FIG. 12A is a workflow of the experiment. FIG. 12B outlines tumor growth as a function of time after DCP infusion. FIGS. 12C-12D show flow cytometry of tumors, indicating robust activation of T cells. FIGS. 12E-12F show flow cytometry of tdLNs, indicating robust infiltration by T cells. FIG. 12G shows reprogramming of the tumor microenvironment and massive expansion of CD8⁺ T cells in tumors of mice that received DCPs engineered to express IL-12 and FLT3L. FIG. 12H shows pie chart based on the same data showed in FIG. 12G, but the data was processed with different inclusion criteria for macrophages.

FIGS. 13A-13H depict differentiation of CD34⁺ human hematopoietic stem progenitor cells into dendritic cell progenitors (DCPs) described herein. FIG. 13A outlines a cell expansion protocol for preparation of the DCPs. FIG. 13B shows the gating strategy for enrichment of DCPs, defined as CD3⁻, CD19⁻, CD335⁻, CD66b⁻, CD10⁻, CD14⁻, CD34⁺ and CD115⁺. FIG. 13C shows % of DCPs present with or without enrichment treatment. FIG. 13D outlines a cell expansion protocol for preparation of the DCPs with or without a stem cell expansion enhancer UM729. FIG. 13E shows effect of addition of UM729 on CD34⁺ human hematopoietic stem progenitor cells obtained from two different donors. FIG. 13F outlines a protocol for in vitro preparation of the DCPs. FIG. 13G shows fluorescence-activated cell sorting (FACS) as CD34⁺ and CD115⁺ cells after cell expansion treatment for 7 days. FIG. 13H shows % of DCPs observed in APCs and other cells, respectively, that were allowed to differentiate for 7 days following cell expansion treatment for 7 days.

FIGS. 14A-14B show two antigen-presentation pathways that were examined for the presence of increased IFNγ and TNFα-producing CMV-specific CD8⁺ T cells. FIG. 14A shows presentation of pp65₄₉₅₋₅₀₄ peptide-loaded HLA-A2, which mimics direct presentation. FIG. 14B shows cross-presentation of the pp65₄₉₅₋₅₀₄ peptide endogenously processed from the native pp65 protein.

FIGS. 15A-15L depict differentiation of CD34⁺ human hematopoietic stem progenitor cells into dendritic cell progenitors (DCPs) described herein. FIG. 15A outlines a cell expansion protocol for lentiviral vector transduced CD34⁺ human hematopoietic stem progenitor cells. FIG. 15B shows flow cytometry of transduced DCPs for determining transgene-expression. FIG. 15C outlines a protocol for preparing DCPs from CD34⁺ human hematopoietic stem progenitor cells transduced with lentiviral vector encoding αGD2-EVIR. FIG. 15D outlines a protocol for assessing the functionality of GD2-EVIR-DCPs by assessing the capacity of DCPs transduced with GD2-EVIR, compared with CTRL dLNGFR, to uptake GD2⁺ or GD2⁻ tumor EVs. FIG. 15E shows uptake of GD2⁺ tumor EVs by GD2-EVIR-transduced DCP-progeny compared to CTRL-transduced DCP-progeny, wherein the uptake was determined by flow cytometry. FIG. 15F outlines a protocol for preparing DCPs from CD34⁺ human hematopoietic stem progenitor cells transduced with lentiviral vector encoding FLT3L or FLT3L/IL-12. FIG. 15G shows flow cytometry of DCPs identified as CD34⁺ and CD115⁺ cells that were transduced with FLT3L-GFP LV FIG. 15H shows ELISA analysis of day 14 culture supernatants for FLT3L production by FLT3L-transduced cells or a mixture of FLT3L⁻ and IL12-transduced DCPs (2:1 ratio). FIG. 15I outlines a protocol for preparing DCPs from CD34⁺ human hematopoietic stem progenitor cells transduced with lentiviral vector encoding IL12 or FLT3L/IL12. FIG. 15J shows flow cytometry of DCPs identified as CD34⁺ and CD115⁺ cells that were transduced with IL12-dLNGFR LV. FIG. 15K shows ELISA analysis of day 14 culture supernatants for IL12 production by IL12-transduced DCP-progeny cells or a mixture of FLT3L/IL12-transduced (2:1 ratio) DCP-progeny cells. FIG. 15L shows antigen-independent IFNγ production by CMV-specific T cells cocultured with DCP-progeny cells, wherein the DCP-progeny cells were either IL12-transduced DCP-progeny cells or a mixture of FLT3L/IL12-transduced (2:1 ratio) DCP-progeny cells.

### DETAILED DESCRIPTION

### Overview

Disclosed herein are synthetically-differentiated dendritic cell progenitors (or DCPs). A synthetically-differentiated dendritic cell progenitor as described herein can be partially differentiated from a host progenitor cell. As such, a synthetically-differentiated dendritic cell progenitor as described herein is a partially differentiated cell. In some embodiments, a synthetically-differentiated dendritic cell progenitor as described herein is not full differentiated.

A synthetically-differentiated dendritic cell progenitor as described herein, upon administration to a host, can naturally differentiate into dendritic cells such as cDC1, cDC2, or immature dendritic cells. As disclosed herein, synthetically-differentiated dendritic cell progenitors of the present disclosure efficiently differentiate into such dendritic cells to a greater extent when administered to a subject, as compared to administration of otherwise comparable dendritic cells such as monocyte-derived dendritic cells (moDCs) or conventional type 1 DC (cDC1) cells. Further, synthetically-differentiated dendritic cell progenitor of the present disclosure are capable of differentiation into dendritic cells in the presence of a tumor, and are thus are capable of differentiation in the presence of inflammation and immune-suppressive cytokines associated with the presence of a tumor.

A synthetically-differentiated dendritic cell progenitor as described herein can be differentiated from a host progenitor cell, e.g., a CD34⁺ human hematopoietic stem progenitor cell. In some embodiments, a synthetically-differentiated dendritic cell progenitor as described herein is a partially differentiated cell. In some embodiments, a synthetically-differentiated dendritic cell progenitor as described herein is not full differentiated. In some embodiments, a synthetically-differentiated dendritic cell progenitor as described herein is capable of differentiating in vitro into an antigen-presenting cells (APCs), cDC2s, monocytes, immature dendritic cells, or combinations thereof. Further, in some embodiments, a synthetically-differentiated dendritic cell progenitor of the present disclosure are capable of differentiation into APCs, cDC2s, monocytes, immature dendritic cells, or combinations thereof in the presence of a tumor, and are thus are capable of differentiation in the presence of inflammation and immune-suppressive cytokines associated with the presence of a tumor.

A synthetically-differentiated dendritic cell progenitor can be engineered to express an interleukin and/or an effector in order to stimulate production of tumor-specific T cells. In some instances, co-expression of an effector such as extracellular vesicle-internalizing receptor (EVIR) or FMS-like tyrosine kinase 3 ligand (FLT3L) along with an interleukin such as IL-12 produces differentiated dendritic cells (e.g., cDC1, cDC2, APCs, monocytes, or immature dendritic cells) that produce tumor-specific T cells that reduce tumor growth, inhibit tumor initiation, or both. Furthermore, the presence of the effector and/or interleukin (whether co-expressed by the differentiated dendritic cell or added exogenously) result in the production of tumor-specific T cells without the need to supply a tumor antigen (e.g., whether exogenously or through expression by the dendritic cell).

Thus, synthetically-differentiated dendritic cell progenitors can be used as a therapeutic to target cancer agnostic to specific tumor antigens. As a result, administration of such synthetically-differentiated dendritic cell progenitors as part of a pharmaceutical composition to a subject having cancer can be used to treat the cancer without any knowledge of antigens expressed on the cancer cell.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, such as plus or minus 10%. Where ranges and/or subranges of values are provided, the ranges and/or subranges include the endpoints of the ranges and/or subranges.

The term “substantially” as used herein refers to a value approaching 100% of a given value. For example, an expression system described herein that does not “substantially” express a transgene in the absence of an inducer can indicate that less than 10% of the transgene (e.g., less than 5%, less than 1%, less than 0.1%, or less than 0.01%) is expressed, relative to an amount of transgene expressed in the presence of the inducer.

The terms “subject,” “individual,” or “patient” can be used interchangeably herein. A “subject” refers to a plant, animal, or microorganism, including, for example, bacteria, viruses, fungi, and protozoa. The subject can be a mammal. A mammal can be any member of the Mammalian class, including but not limited to a human, a non-human primate such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like.

The term “host” and “donor” are used interchangeably herein to refer to an organism in which a progenitor cell is isolated from. A host can be a mammal as described herein. Where a progenitor cell is isolated from a “host” and differentiated into a therapeutic for administration to a “subject,” the host and subject do not have to be same class, genus, or species of animal.

The term “in vivo” refers to an event that takes place in a subject's body.

The term “in vitro” refers to an event that takes place outside of a subject's body. In vitro assays can encompass cell-based assays in which living or dead cells can be employed. In vitro assays can also encompass a cell-free assay in which no intact cells can be employed.

### Dendritic Cell Progenitors

Disclosed herein are dendritic cell progenitors that are synthetically partially differentiated from a host progenitor cell. As disclosed herein, a host progenitor cell includes a progenitor cell from a host that is capable of partial or full differentiation. In some instances, a host progenitor cell can be isolated from a host such as a mammal. In some embodiments, a progenitor cell can be a progenitor cell isolated from bone marrow or blood, such as a hematopoietic stem or progenitor cell. Examples of hematopoietic progenitor cells include hematopoietic stem cells, multipotent progenitors, and myeloid progenitor cells and lymphoid progenitor cells. In some embodiments, a progenitor cell can be a dedifferentiated cell such as an induced pluripotent stem cell or a neural progenitor cell.

As disclosed herein, a synthetically partially differentiated dendritic cell progenitor can be prepared from a host progenitor cell by contacting the host progenitor cell with a synthetic medium to induce partial differentiation. In some embodiments, the resulting synthetically partially differentiated dendritic cell progenitor is capable of additional differentiation into a dendritic cell (i.e., the dendritic progenitor cell is not fully differentiated).

Also disclosed herein is a host progenitor cell comprising a human progenitor cell. In some embodiments, the human progenitor cell can be isolated from bone marrow or blood, such as cord-blood CD34⁺ progenitor cell. In some embodiments, the human progenitor cell is capable of undergoing partial differentiation into a dendritic cell progenitor. Accordingly, in some embodiments, the dendritic cell progenitor can be prepared by contacting the human progenitor cell (e.g., CD34⁺ progenitor cell) with a synthetic medium, as described herein, to induce partial differentiation. In some embodiments, the dendritic cell progenitor derived from the human progenitor cell is capable of undergoing in vitro differentiation into antigen-presenting cells (APCs), cDC2s, monocytes, immature dendritic cells, or combinations thereof.

A synthetic medium for differentiation can include an effective amount of an effector sufficient to induce partial differentiation of the host progenitor cell. In some embodiments, the synthetic medium comprises at least about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, 30 ng/mL, 31 ng/mL, 32 ng/mL, 33 ng/mL, 34 ng/mL, 35 ng/mL, 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 51 ng/mL, 52 ng/mL, 53 ng/mL, 54 ng/mL, 55 ng/mL, 56 ng/mL, 57 ng/mL, 58 ng/mL, 59 ng/mL, 60 ng/mL, 61 ng/mL, 62 ng/mL, 63 ng/mL, 64 ng/mL, 65 ng/mL, 66 ng/mL, 67 ng/mL, 68 ng/mL, 69 ng/mL, 70 ng/mL, 71 ng/mL, 72 ng/mL, 73 ng/mL, 74 ng/mL, 75 ng/mL, 76 ng/mL, 77 ng/mL, 78 ng/mL, 79 ng/mL, 80 ng/mL, 81 ng/mL, 82 ng/mL, 83 ng/mL, 84 ng/mL, 85 ng/mL, 86 ng/mL, 87 ng/mL, 88 ng/mL, 89 ng/mL, 90 ng/mL, 91 ng/mL, 92 ng/mL, 93 ng/mL, 94 ng/mL, 95 ng/mL, 96 ng/mL, 97 ng/mL, 98 ng/mL, 99 ng/mL, 100 ng/mL, 101 ng/mL, 102 ng/mL, 103 ng/mL, 104 ng/mL, 105 ng/mL, 106 ng/mL, 107 ng/mL, 108 ng/mL, 109 ng/mL, 110 ng/mL, 111 ng/mL, 112 ng/mL, 113 ng/mL, 114 ng/mL, 115 ng/mL, 116 ng/mL, 117 ng/mL, 118 ng/mL, 119 ng/mL, 120 ng/mL, 121 ng/mL, 122 ng/mL, 123 ng/mL, 124 ng/mL, 125 ng/mL, 126 ng/mL, 127 ng/mL, 128 ng/mL, 129 ng/mL, 130 ng/mL, 131 ng/mL, 132 ng/mL, 133 ng/mL, 134 ng/mL, 135 ng/mL, 136 ng/mL, 137 ng/mL, 138 ng/mL, 139 ng/mL, 140 ng/mL, 141 ng/mL, 142 ng/mL, 143 ng/mL, 144 ng/mL, 145 ng/mL, 146 ng/mL, 147 ng/mL, 148 ng/mL, 149 ng/mL, 150 ng/mL, 151 ng/mL, 152 ng/mL, 153 ng/mL, 154 ng/mL, 155 ng/mL, 156 ng/mL, 157 ng/mL, 158 ng/mL, 159 ng/mL, 160 ng/mL, 161 ng/mL, 162 ng/mL, 163 ng/mL, 164 ng/mL, 165 ng/mL, 166 ng/mL, 167 ng/mL, 168 ng/mL, 169 ng/mL, 170 ng/mL, 171 ng/mL, 172 ng/mL, 173 ng/mL, 174 ng/mL, 175 ng/mL, 176 ng/mL, 177 ng/mL, 178 ng/mL, 179 ng/mL, 180 ng/mL, 181 ng/mL, 182 ng/mL, 183 ng/mL, 184 ng/mL, 185 ng/mL, 186 ng/mL, 187 ng/mL, 188 ng/mL, 189 ng/mL, 190 ng/mL, 191 ng/mL, 192 ng/mL, 193 ng/mL, 194 ng/mL, 195 ng/mL, 196 ng/mL, 197 ng/mL, 198 ng/mL, 199 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, 250 ng/mL, 255 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, 400 ng/mL, 405 ng/mL, 410 ng/mL, 415 ng/mL, 420 ng/mL, 425 ng/mL, 430 ng/mL, 435 ng/mL, 440 ng/mL, 445 ng/mL, 450 ng/mL, 455 ng/mL, 460 ng/mL, 465 ng/mL, 470 ng/mL, 475 ng/mL, 480 ng/mL, 485 ng/mL, 490 ng/mL, 495 ng/mL, 500 ng/mL, 505 ng/mL, 510 ng/mL, 515 ng/mL, 520 ng/mL, 525 ng/mL, 530 ng/mL, 535 ng/mL, 540 ng/mL, 545 ng/mL, 550 ng/mL, 555 ng/mL, 560 ng/mL, 565 ng/mL, 570 ng/mL, 575 ng/mL, 580 ng/mL, 585 ng/mL, 590 ng/mL, 595 ng/mL, 600 ng/mL, 605 ng/mL, 610 ng/mL, 615 ng/mL, 620 ng/mL, 625 ng/mL, 630 ng/mL, 635 ng/mL, 640 ng/mL, 645 ng/mL, 650 ng/mL, 655 ng/mL, 660 ng/mL, 665 ng/mL, 670 ng/mL, 675 ng/mL, 680 ng/mL, 685 ng/mL, 690 ng/mL, 695 ng/mL, 700 ng/mL, 705 ng/mL, 710 ng/mL, 715 ng/mL, 720 ng/mL, 725 ng/mL, 730 ng/mL, 735 ng/mL, 740 ng/mL, 745 ng/mL, 750 ng/mL, 755 ng/mL, 760 ng/mL, 765 ng/mL, 770 ng/mL, 775 ng/mL, 780 ng/mL, 785 ng/mL, 790 ng/mL, 795 ng/mL, 800 ng/mL, 805 ng/mL, 810 ng/mL, 815 ng/mL, 820 ng/mL, 825 ng/mL, 830 ng/mL, 835 ng/mL, 840 ng/mL, 845 ng/mL, 850 ng/mL, 855 ng/mL, 860 ng/mL, 865 ng/mL, 870 ng/mL, 875 ng/mL, 880 ng/mL, 885 ng/mL, 890 ng/mL, 895 ng/mL, 900 ng/mL, 905 ng/mL, 910 ng/mL, 915 ng/mL, 920 ng/mL, 925 ng/mL, 930 ng/mL, 935 ng/mL, 940 ng/mL, 945 ng/mL, 950 ng/mL, 955 ng/mL, 960 ng/mL, 965 ng/mL, 970 ng/mL, 975 ng/mL, 980 ng/mL, 985 ng/mL, 990 ng/mL, 995 ng/mL, or 1000 ng/mL of an effector.

In some embodiments, the synthetic medium can comprise a mixture of effectors. For example, a synthetic medium can comprise at least 2, 3, 4, 5, 6, 7, 8, 9, or more than 10 effectors. In some instances, an effector can be a cytokine. Examples of cytokines include IL-1, TNF-α, TPO, SCF, IL-3, IL-6, IL-12, IL-4, CXCL8, FLT3L, GM-CSF, IFNa, PGE2, retronectin, UM729, and G-CSF. In some embodiments, the synthetic medium comprises a mixture of GM-CSF and FLT3L. In some embodiments, the IFNa is IFNa2b. In some embodiments, the synthetic medium comprises a mixture of GM-CSF, FLT3L, SCF, and IFNa2b. In some embodiments, the synthetic medium does not comprise UM729.

A synthetically partially differentiated dendritic cell progenitor differentiated from a host progenitor cell using synthetic medium as described herein differs from naturally-occurring dendritic progenitor cells or mature dendritic cells. For example, a synthetically partially differentiated dendritic cell progenitor is capable of differentiation into mature dendritic cells (e.g., cDC1 or cDC2) or immature dendritic cells under conditions in which naturally-occurring dendritic cells are unable to be differentiated. For example, a synthetically partially differentiated dendritic cell progenitor is capable of differentiation into a mature dendritic cell in the presence of inflammatory or immune suppressive cytokines, such as those secreted by a tumor. For example, a synthetically partially differentiated dendritic cell progenitor that is derived from human progenitor cell is capable of differentiating into antigen-presenting cell (APC), cDC2, monocyte, immature dendritic cell, or a combination thereof. Accordingly, in some embodiments, a synthetically partially differentiated dendritic cell progenitor is capable of undergoing differentiation into APCs, cDC2s, monocytes, immature dendritic cells, or combinations thereof in the presence of inflammatory or immune suppressive cytokines, such as those secreted by a tumor.

A synthetically partially differentiated dendritic cell progenitor as disclosed herein can present with a particular phenotype that differs from a naturally-occurring dendritic cell progenitor. For example, a synthetically partially differentiated dendritic cell progenitor can have a flow cytometry phenotype that is one or more of: CD115⁺, CD11b-neg, CD11c-neg, MHCII-neg, CD45R/B220-neg, cKIT⁻ neg/low, and Clec9A-neg. Alternatively, in some embodiments, a synthetically partially differentiated dendritic cell progenitor can have a flow cytometry phenotype that is one or more of: CD3⁻, CD19⁻, CD335⁻, CD66b⁻, CD10⁻, CD14⁻, CD34⁺, and CD115⁺.

In some embodiments, a synthetically partially differentiated dendritic cell progenitor can be an engineered dendritic cell progenitor. For example, a synthetically partially differentiated dendritic cell progenitor can be engineered to co-express a transgene that, when expressed, works in concert with a dendritic cell differentiated from the synthetically partially differentiated dendritic cell progenitor to activate a subject's immune system. For example, an engineered dendritic cell progenitor as described herein can be engineered to co-express an interleukin, an effector, or both. An interleukin that can be co-expressed in an engineered dendritic cell progenitor can include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, or IL-40. In some embodiments, the interleukins described herein comprises interleukins derived from the same species as the host. For example, the interleukins described herein can comprise human interleukins where the dendritic cell progenitor is derived from a human cell. An effector that can be co-expressed in an engineered dendritic progenitor cell can include an internalizing receptor such as extracellular vesicle-internalizing receptor (EVIR); or a cytokine such as IL-1, TNF-α, IL-6, IL-12, IL-2, CXCL8, FLT3L, GM-CSF, IFNa, PGE2, retronectin, and G-CSF. In some embodiments, the effectors comprise effectors from the same species as the dendritic cell progenitor. For example, the effectors described herein comprises human effectors where the dendritic cell progenitor is derived from a human cell.

A synthetically partially differentiated dendritic cell progenitor can be included in an in vitro cell composition. In some instances, the in vitro cell composition can be used to prepare functional mature dendritic cells (e.g., antigen-presenting cell (APC), monocyte, immature dendritic cell, cDC1 or cDC2) in vitro for use as a therapeutic. In some instances, the in vitro cell composition can be included in a pharmaceutical composition further comprising a pharmaceutically-acceptable excipient, diluent, or carrier. In some embodiments, a pharmaceutical formulation can comprise an excipient. An excipient includes an excipient described in the Handbook of Pharmaceutical Excipients, American Pharmaceutical Association (1986). In some embodiments, an excipient can include a buffering agent, a preservative, a stabilizer, a binder, a compaction agent, a lubricant, a chelator, a dispersion enhancer, a disintegration agent, a flavoring agent, a sweetener, a coloring agent. A diluent can include water; glycerol; methanol; ethanol; an aqueous acid such as acetic acid, citric acid, maleic acid, hydrochloric acid, phosphoric acid, nitric acid, sulfuric acid, or similar; an alkaline metal phosphates such as calcium phosphate; an alkaline metal sulphates such as calcium sulphate; an alkaline metal carbonates such as calcium carbonate; a cellulose derivative such as cellulose, microcrystalline cellulose, cellulose acetate, mannitol, fructose, dextrose, magnesium oxide, dextrin, glyceryl palmitostearate, caoline, lactose, maltose, simethicone, sorbitol, starch, pregelatinized starch, talc, lactitol, xylitol; and/or anhydrates, hydrates and/or pharmaceutically acceptable derivatives thereof or combinations thereof.

A pharmaceutical composition containing an in vitro cell composition as described herein can be administered to a subject to treat a condition described herein. In some embodiments, a pharmaceutical composition can further comprise an interleukin, an effector or both. An interleukin that can be included in a pharmaceutical composition can include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, or IL-40. In some embodiments, the interleukins described herein comprises interleukins derived from the same species as the host. For example, the interleukins described herein can comprise human interleukins where the dendritic cell progenitor is derived from a human cell. An effector that can be included in a pharmaceutical composition can include an internalizing receptor such as extracellular vesicle-internalizing receptor (EVIR); or a cytokine such as IL-1, TNF-α, IL-6, IL-12, IL-2, CXCL8, FLT3L, IFNa, and GM-CSF. In some embodiments, the effectors comprise effectors from the same species as the dendritic cell progenitor. For example, the effectors described herein comprises human effectors where the dendritic cell progenitor is derived from a human cell.

### Methods of Making a Dendritic Progenitor Cell

Also disclosed herein are methods of making a synthetically partially-differentiated dendritic cell progenitor. As disclosed herein, a synthetically partially-differentiated dendritic cell progenitor can be prepared by contacting a host progenitor cell with a synthetic medium as described herein. In some embodiments, a host progenitor cell can be expanded prior to contacting with the synthetic medium. Expansion can include culturing host progenitor cells isolated from a sample from the host (e.g., bone marrow or blood) in an expansion medium. Such an expansion medium can include 10% FBS, 1% L-glutamine, 100 ng/ml SCF, 40 ng/ml TPO, 50 ng/ml FLT3L, 30 ng/ml IL-3, 30 ng/ml IL-6, and 30 ng/ml IL-1b. Similarly, expansion of human progenitor cells that were isolated from a sample from the human (e.g., cord-blood CD34⁺ progenitors) may include culturing the human progenitor cells in an expansion medium comprising FLT3L, SCF, IL3, IL6 and TPO.

Partial differentiation of the host progenitor cells (e.g., expanded hematopoietic stem/progenitor cells) into synthetically partially-differentiated dendritic cell progenitors can be performed by culturing the host progenitor cells with synthetic medium as described herein (e.g., medium with cytokines or effectors). In some instances, the partial differentiation can be performed for a time period of at least 12 hours, 24 hours, 36 hours, 48 hours, 60 hours, 72 hours, 84 hours, 96 hours, 108 hours, at least 120 hours, at least 144 hours, at least 168 hours, at least 192 hours, at least 216 hours, or at least 240 hours. After differentiation, the cells can be selected based on phenotype and isolated to produce purified synthetically partially-differentiated dendritic cell progenitors.

### Methods of Treatment

Also disclosed herein are methods of treating a condition in a subject in need thereof and pharmaceutical compositions for use in treatment of a condition. In some embodiments, a method of treatment can comprise administering to a subject a synthetically partially-differentiated dendritic cell progenitor as described herein, an in vitro cell composition containing a synthetically partially-differentiated dendritic progenitor cells as described herein, or a pharmaceutical composition containing synthetically partially-differentiated dendritic cell progenitors described herein. In some embodiments, a method of treatment can comprise administering to a subject a mature dendritic cell differentiated from a synthetically partially-differentiated dendritic cell progenitor as described herein (e.g., differentiated in vitro), or in vitro cell compositions or pharmaceutical compositions comprising a mature dendritic cell differentiated from a synthetically partially-differentiated dendritic cell progenitor as described herein.

Administering to a subject can include administration by inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof.

In some embodiments, a method of treatment can include treatment of a cancer in a subject. Examples of cancer can include acute lymphoblastic leukemia, acute myeloid leukemia, adrenocortical carcinoma, basal-cell carcinoma, bile duct cancer, bladder cancer, bone tumor, osteosarcoma/malignant fibrous histiocytoma, brainstem glioma, brain tumor, cerebellar astrocytoma, cerebral astrocytoma/malignant glioma, ependymoma, medulloblastoma, supratentorial primitive neuroectodermal tumors, breast cancer, bronchial adenomas/carcinoids, Burkitt's lymphoma, carcinoid tumor, cervical cancer, chronic lymphocytic leukemia, chronic myelogenous leukemia, chronic myeloproliferative disorders, colon cancer, cutaneous T-cell lymphoma, endometrial cancer, ependymoma, esophageal cancer, Ewing's sarcoma, intraocular melanoma, retinoblastoma, gallbladder cancer, gastric cancer, gastrointestinal carcinoid tumor, gastrointestinal stromal tumor, germ cell tumor, glioma, childhood visual pathway and hypothalamic, Hodgkin lymphoma, melanoma, islet cell carcinoma, Kaposi sarcoma, renal cell cancer, laryngeal cancer, leukemia, lymphomas, mesothelioma, neuroblastoma, non-Hodgkin lymphoma, oropharyngeal cancer, osteosarcoma, ovarian cancer, pancreatic cancer, parathyroid cancer, pharyngeal cancer, pituitary adenoma, plasma cell neoplasia, prostate cancer, renal cell carcinoma, retinoblastoma, sarcoma, testicular cancer, thyroid cancer, and uterine cancer.

In some embodiments, administration is sufficient to reduce the number and/or size of cancer cells. For example, where the cancer is a solid tumor cancer, the administration can result in a reduction in tumor size and/or inhibition of tumor initiation.

A method of treating cancer need not require knowledge of tumor antigens. In some embodiments, administration of a composition containing synthetically-partially differentiated dendritic cells or mature dendritic cells differentiated therefrom does not require administration of, or co-expression of, an antigen expressed on a cell of the cancer. Rather, administration of a composition containing synthetically-partially differentiated dendritic cells or mature dendritic cells differentiated therefrom results in target agnostic treatment of the cancer. In some embodiments, the effector is not expressed on a cell of the cancer.

### EXAMPLES

For a better understanding of the present disclosure and of its many advantages, the following examples are given by way of illustration and without limiting the scope of this disclosure.

### Example 1: Generation of Dendritic Cell Progenitors (DCPs) from Mouse Hematopoietic Cells

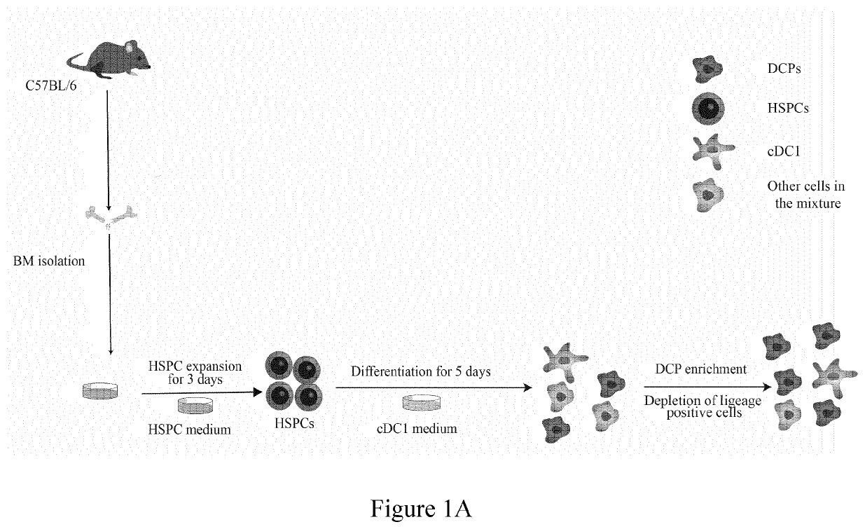

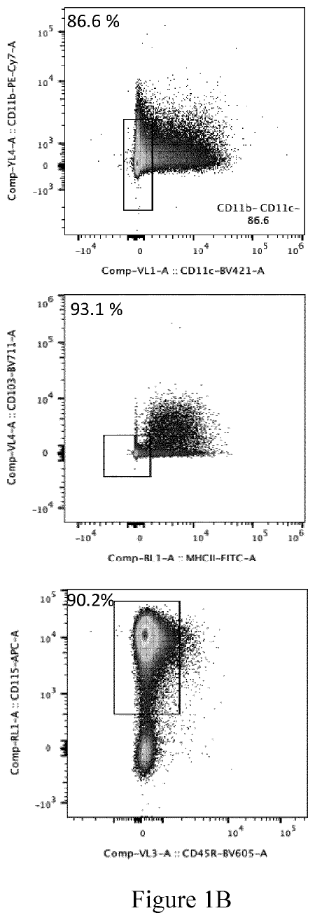

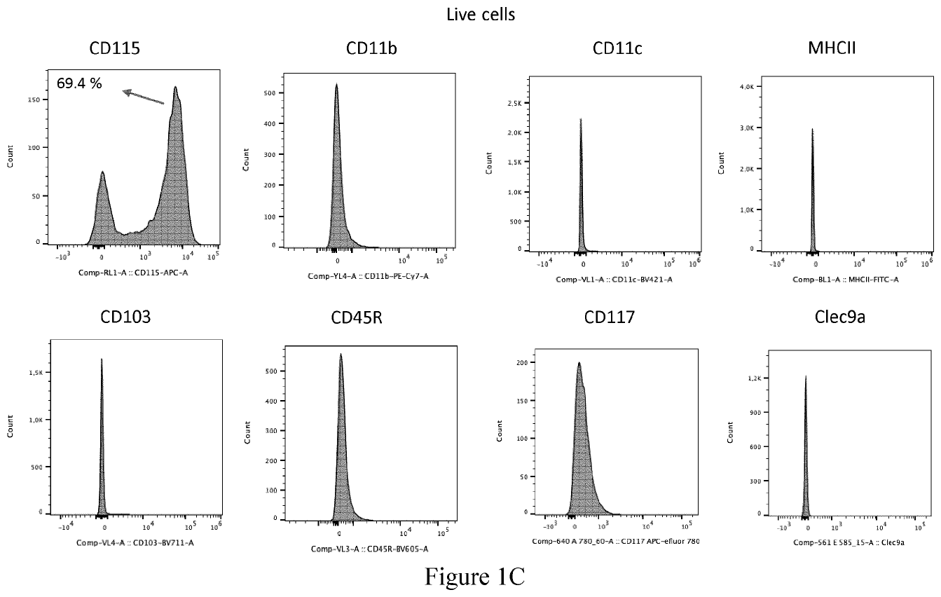

Dendritic progenitor cells (DCPs) were prepared from mouse hematopoietic stem/progenitor cells (FIG. 1A) using a two-step protocol.

### Step 1: Expansion of Hematopoietic Stem Progenitor Cells

The expansion step uses medium that supports hematopoietic stem/progenitor cell (HSPC) maintenance and expansion. This medium contains RPMI 1640 medium with 10% FBS, 1% L-glutamine, 1% Penstrep (called complete RPMI medium) supplemented with 100 ng/ml SCF, 40 ng/ml TPO, 50 ng/ml FLT3L, 30 ng/ml IL-3, 30 ng/ml IL-6, and 30 ng/ml IL-1b.

Total mouse bone marrow (BM) cells were isolated from long bones of C57BL/6 mice and red blood cells (RBCs) were depleted by incubation in 5-10 ml of RBC lysis buffer (Cat No. R7767-100 ML) for 5 minutes. The BM cells were then passed through a 70 μm cell strainer (Cat No. 352350), washed in complete RPMI medium, and resuspended and plated (1-3×10⁶ cells/ml) in the HSPC medium described above. The cells were cultured in HSPC medium for 2 days. The floating cells were then harvested and replated at the same density in HSPC medium for 1 additional day to further remove the remaining adherent cells.

### Step 2: Differentiation of HSCPs to DCPs

The floating HSPCs (end of day 3 of step 1) were harvested and washed once in complete RPMI medium. The harvested cells were then cultured in medium that supports cDC1 differentiation. This medium contains complete RPMI medium supplemented with 200 ng/ml FLT3L and 5 ng/ml GM-CSF. The cells were plated at a density of 1-3×10⁶ cells/ml. After 3 days of differentiation in cDC1 medium, an equal volume of cDC1 medium was added to each well. After 2 additional days (end of day 5 of step 2), the cell culture contained 20-50% of cells, which are called DCPs. The DCPs could then be further enriched by depleting the main lineage-positive cells (CD5⁺, CD45R/B220⁺, CD11b⁺, CD19⁺, Ly6C/G⁺, TER119⁺) through negative selection. The final purity of DCPs after negative selection at the end of the protocol (end of day 8 of steps 1 and 2 combined) was 60-90% (FIG. 1B).

After enrichment, the DCPs present the following phenotype by flow cytometry analysis: CD115⁺, CD11b-neg, CD11c-neg, MHCII-neg, CD45R/B220-neg, cKIT⁻ neg/low, and Clec9A-neg (FIG. 1C). These DCPs differ from common dendritic cell progenitors (CDPs) as the latter express Clec9A, and differ from pre-cDC1 as the latter express Clec9A and CD11c.

This example thus demonstrates a facile protocol for the efficient generation and enrichment of DCPs from mouse BM.

### Example 2: Superior In Vivo Differentiation of DCPs into Conventional Type I and II DCs (cDC1 and cDC2)

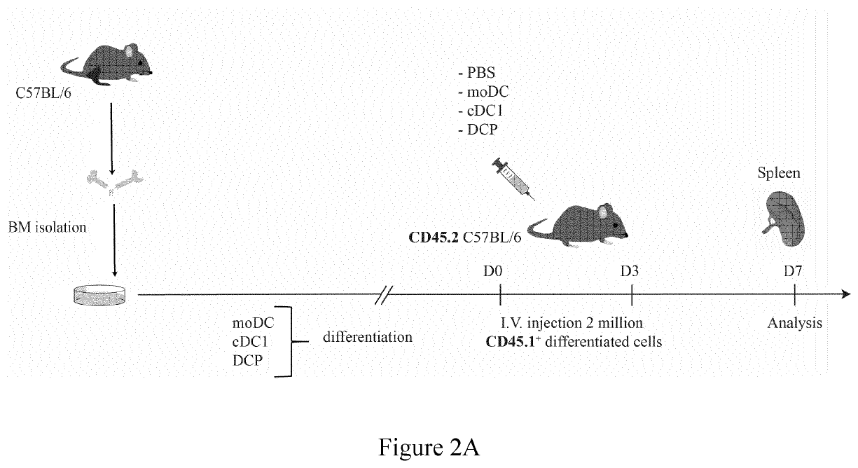

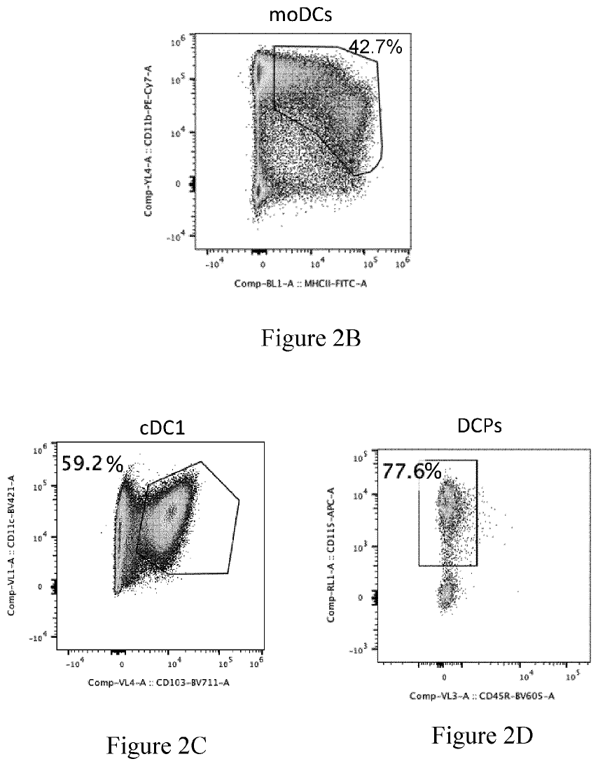

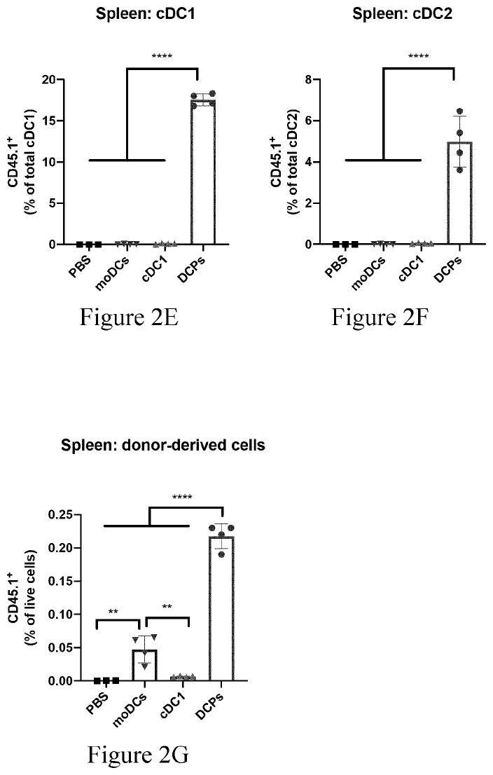

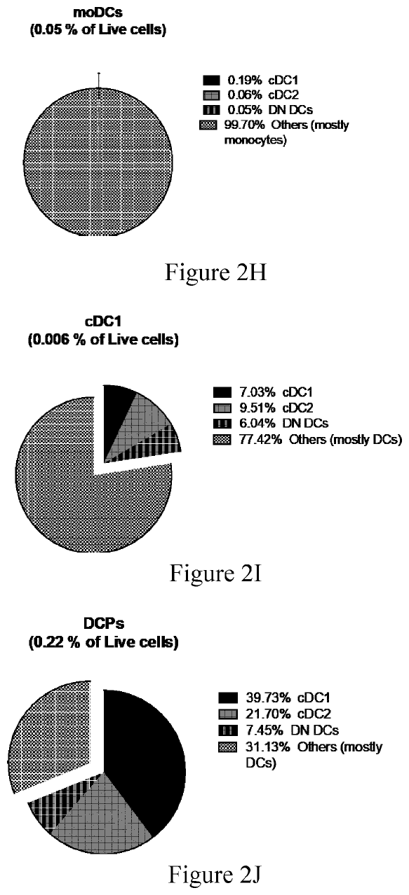

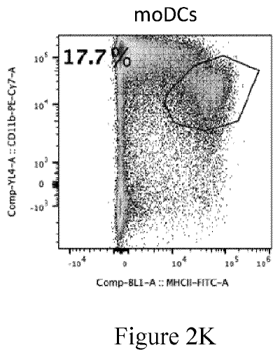

The ability of different types of DCs to form cDC1 in tumor-free mice was next investigated (FIGS. 2A-2K).

BM cells were isolated from CD45.1 mice to enable the tracking of donor-derived cells in congenic CD45.2 C57BL/6 mice. Different types of DCs were generated by differentiating mouse BM cells in different differentiation media (FIG. 2A). 3 types of DCs were compared:
(i) Monocyte-derived dendritic cells (moDCs) were generated using an established protocol involving incubating BM cells in complete RPMI medium supplemented with 100 ng/ml GM-CSF and 40 ng/ml IL4 (referred to as moDC medium). BM cells were cultured at a density of 2-3×10⁶ cell/ml for 2 days in moDC medium. The non-adherent and loosely adherent cells were then harvested, replated at a density of 2-3×10⁶/ml, and cultured in moDC medium for 6 additional days (fresh moDC medium was added once). FIGS. 2B and 2K show the phenotype of the moDC culture at the end of the differentiation protocol (end of day 8; note that moDC cultures may contain some macrophages) and before injection into recipient CD45.2 mice. FIGS. 2B and 2K illustrate two gating strategies; FIG. 2K identifies MHCII-high moDCs, whereas FIG. 2B shows MHCII+ moDCs with MHCII levels ranging from low to high.
(ii) Conventional type I DCs (cDC1) are professional antigen-presenting cells, whose important roles in anti-tumor immunity have been recently demonstrated. cDC1 cells may thus serve as a novel DC type for vaccination applications. cDC1 cells were generated. Briefly, BM cells were directly plated at a density of 2×10⁶ cells/ml in cDC1 medium. The cells were cultured for a total of 16 days, by adding 1 ml of fresh cDC1 medium every 3-4 days. FIG. 2C shows the phenotype of cDC1 cells at the end of the differentiation protocol (end of day 16) and before injection into recipient CD45.2 mice.
(iii) DCPs were generated as shown in Example 1. FIG. 2D shows the phenotype of DCPs at the end of the protocol (end of day 8) and before injection into recipient CD45.2 mice.

Each type of DC, prepared as described above, was infused systemically (via the tail vein) in syngeneic CD45.2 mice, without any prior conditioning of the mouse. Two DC doses of 2×10⁶ cells (in 200 ul of PBS) were administered 3 days apart, and the recipient mice (4 mice per condition) were sacrificed 4 days after the second DC injection to analyze the phenotype of donor-derived CD45.1⁺ cells (FIG. 2A). Control mice (3 mice) received PBS. Spleens were smashed thoroughly on a 70 μm cell strainer and RBCs were depleted using an RBS lysis buffer; the cells were then washed in PBS with 10% FBS and 2 mM EDTA before analysis. The data showed substantial chimerism among cDCs (both cDC1 and cDC2; more than 15% of donor-derived cells were cDC1) in mice that received DCPs, while there were negligible amounts of donor-derived cDCs in mice infused with moDCs or cDC1 cells (FIGS. 2E-2F). In addition to substantial cDC chimerism following DCP injection, DCP-treated mice had higher frequency of donor-derived cells (FIG. 2G), which indicates superior longevity of injected DCPs compared to other types of DCs.

The analysis of CD45.1⁺ splenocytes revealed that moDC-derived cells did not contain cDCs or double-negative (DN; CD11b-neg, CD11c⁺, MHC-II⁺, CD8a-neg) immature DCs (FIG. 2H). Also, only a minority of the cDC1-derived cells were either cDC1, cDC2 or DN DCs (FIG. 2I). Importantly, the vast majority of the DCP-derived cells derived were cDC1, cDC2 or DN DCs (FIG. 2J).

Statistical analysis: e-g) One-way ANOVA with Tukey's multiple comparison test. P values are coded as *: P<0.05; **: P<0.01; ***: P<0.001; and ****: P<0.0001.

This example thus demonstrates that DCPs efficiently generate cDC1, cDC2 and immature DCs in mice, whereas traditional moDCs or mature cDC1 fail to do so.

### Example 3: DCPs Differentiate into Intratumoral cDC1 and cDC2 after Systemic Injection

Example 2 showed that DCPs, unlike moDCs and mature cDC1, efficiently differentiate into cDC1 and cDC2 after systemic injection in tumor-free mice. The ability of DCPs to differentiate in the presence of inflammation and upregulation of immune-suppressive cytokines in tumor-bearing mice. DCPs were then infused in tumor-bearing mice to study migration and phenotype of donor-derived cells in both spleen and tumor.

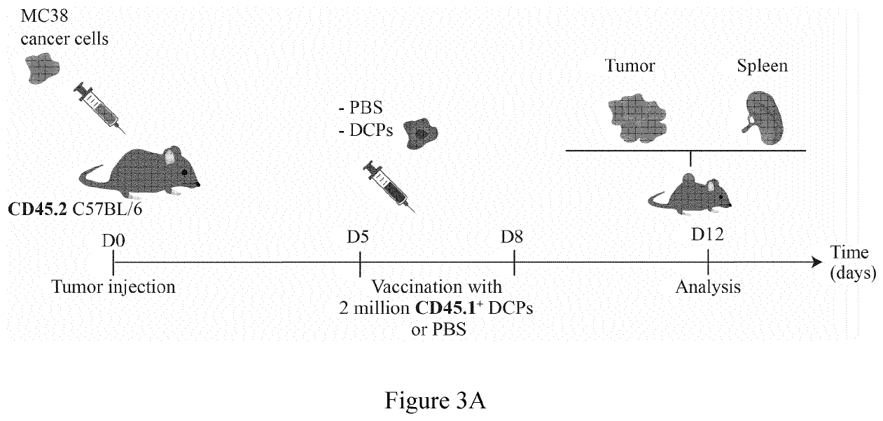

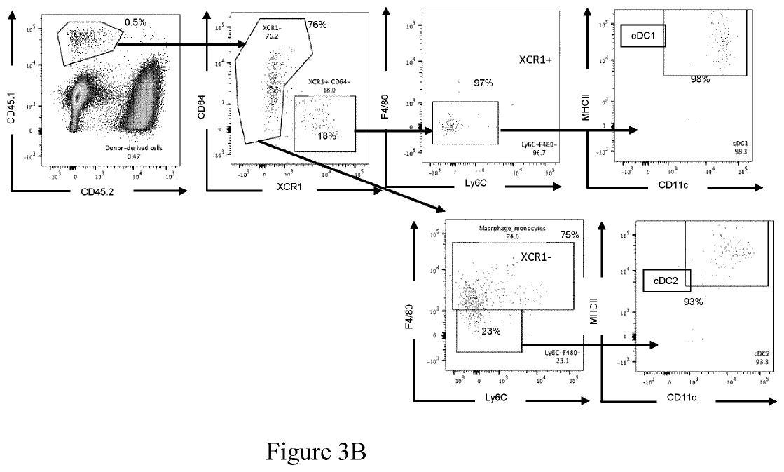

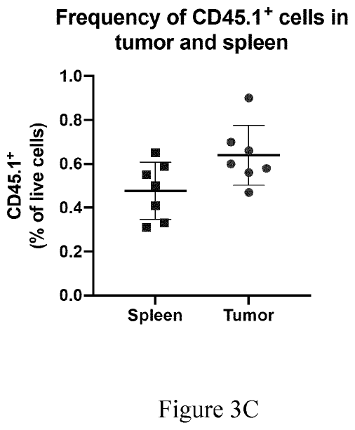

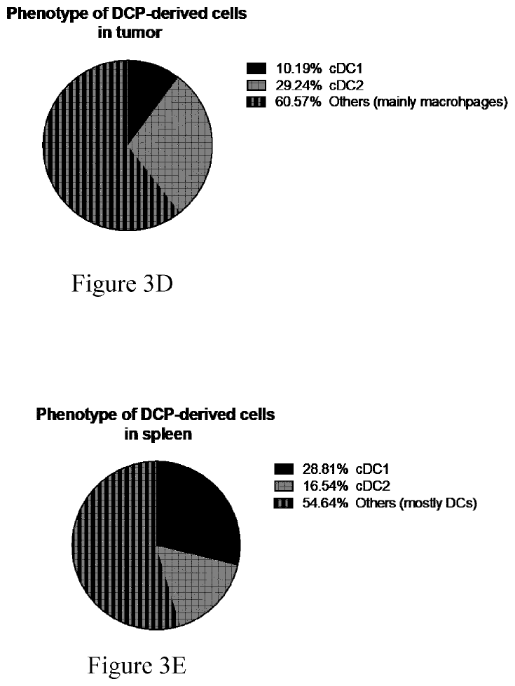

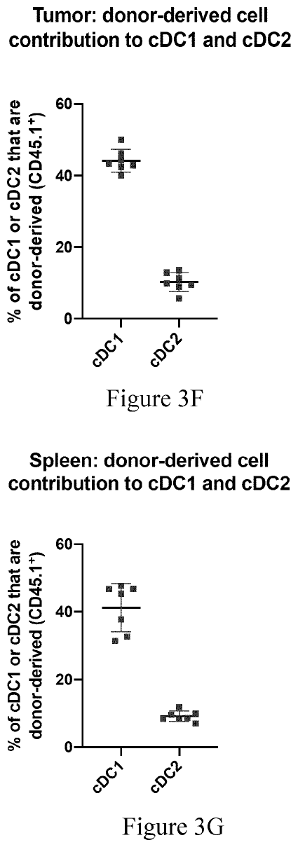

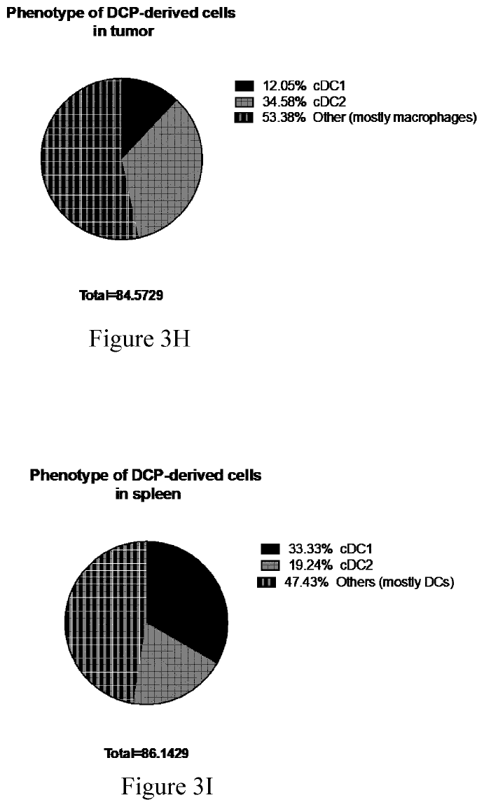

MC38 cancer cells (5×10⁵ cells) were injected into the right flank of CD45.2 mice (8 mice per condition). The tumor-bearing mice were then infused with DCPs generated and enriched according to the two-step protocol shown in Example 1. Enriched DCPs (2×10⁶ cells in 200 ul of PBS) were injected systemically through tail vein at both day 5 and day 8 post-tumor injection. Control mice (8 mice) received PBS. The mice were sacrificed 4 days after the second DCP injection. The spleen and tumor of each mouse were analyzed for the presence of donor-derived (CD45.1⁺) cells. FIG. 3A shows the workflow of the experiment. At the end of the experiment, spleens were smashed thoroughly on a 70 μm cell strainer and RBCs were depleted using an RBS lysis buffer; the cells were then washed in PBS with 10% FBS and 2 mM EDTA before analysis. Single cells from tumors were isolated using conventional enzymatic digestion.

FIG. 3B shows the gating strategy for the identification of intratumoral donor-derived cDC1 and cDC2. Flow cytometry analysis of tumor-derived cells and splenocytes revealed that donor CD45.1⁺ cells infiltrate the tumor and spleen after systemic injection (FIG. 3C) and efficiently differentiate into cDCs (FIGS. 3D-3E and 3H-3I). Almost half of all cDC1 were donor-derived (CD45.1⁺) in both tumor and spleen (FIGS. 3F-3G), which shows substantial chimerism of the most professional antigen-presenting cells after DCP infusion in non-conditioned mice. Chimerism of cDC2 was lower but well detectable in all mice.

Thus, this example demonstrates that DCPs efficiently generate cDC1 and cDC2 in tumor and spleen of tumor-bearing mice without prior conditioning of the mouse.

### Example 4: Identification of Cytokines that Enable DCP Differentiation into cDC1 with Co-Stimulatory Capacity

Cytokines were screened in an in vitro screening study for the ability to retain the differentiation potential of DCPs into cDC1 while enabling the T-cell co-stimulatory capacity of the resulting cDC1 in co-culture with T cells.

To study differentiation, DCPs were cultured in cDC1 medium supplemented with selected interleukins (ILs) at different concentrations. To study T cell co-stimulation, the same cytokines were used in co-cultures of antigen-loaded cDC1-like cells and antigen-specific T cells.

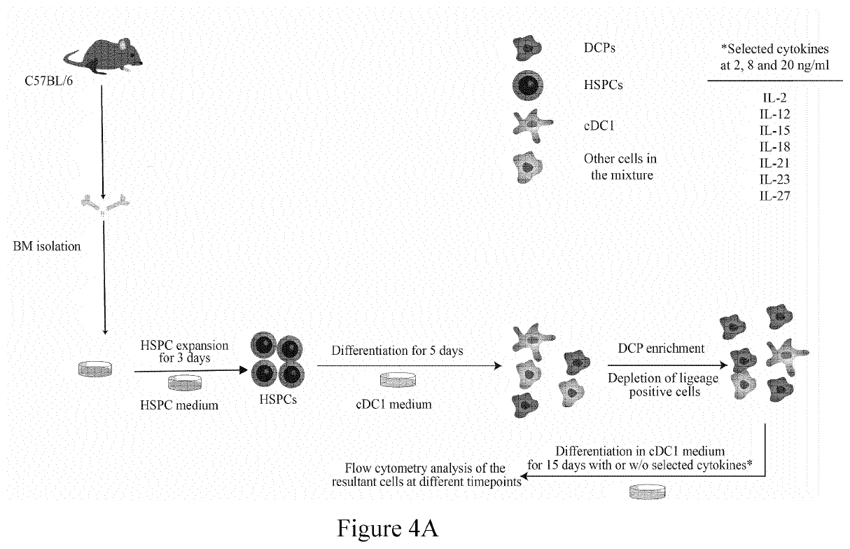

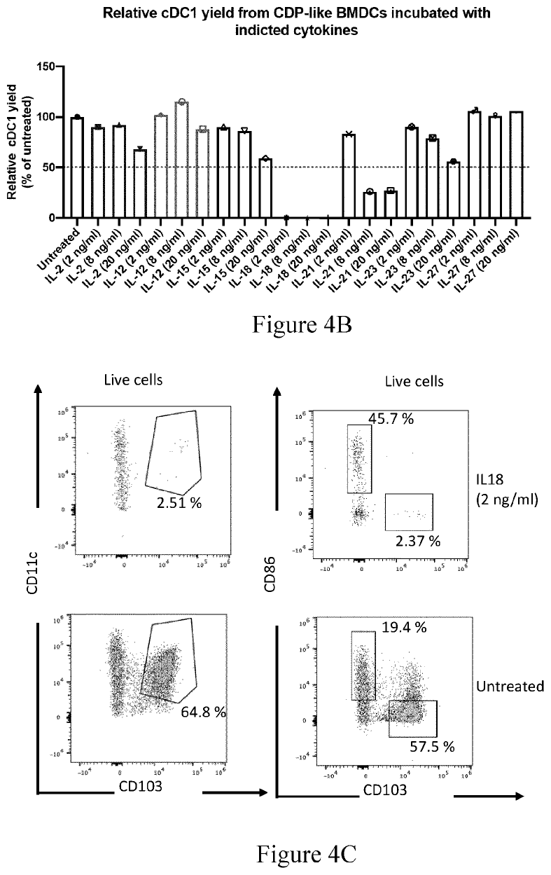

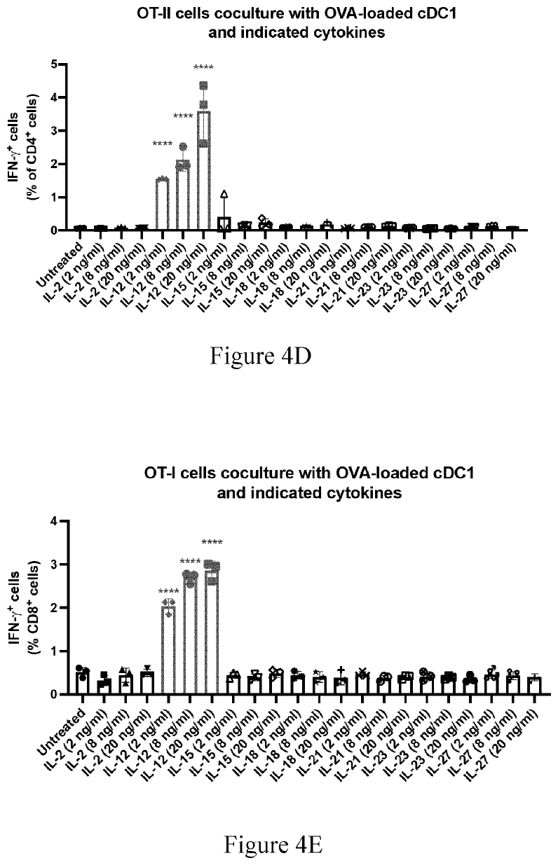

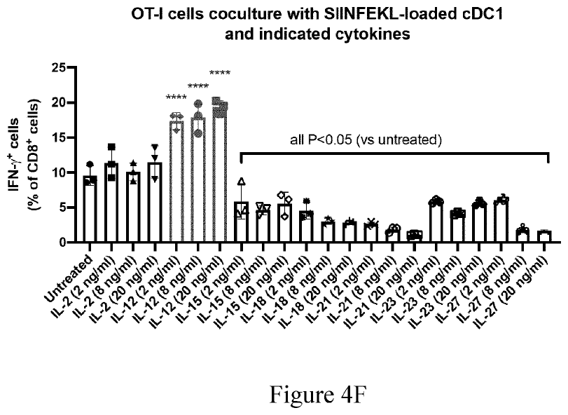

DCPs were generated as explained in Example 1. Enriched DCPs were cultured in cDC1 medium supplemented with various ILs (IL-2, IL-12, IL-15, IL-18, IL-21, IL-23, or IL-27) at the indicated concentrations, ranging 2-20 ng/ml. One ml of IL-supplemented cDC1 medium was added to DCPs every 3-4 days for 15 days (FIG. 4A). The cells were analyzed for the presence of cDC1 (identified as CD11c⁺ CD103⁺) after 15 days and were compared with DCPs cultured in cDC1 medium without ILs. Flow cytometry analysis demonstrated that whereas IL-18 and IL-21 induced premature activation of DCPs (indicated by increased proportion of CD86⁺ CD103-neg cells) and compromised the differentiation of DCPs to cDC1 (indicated by decreased proportion of CD103⁺ CD86-neg cells), other ILs (IL-2, IL-12, IL-15, IL-23 and IL-27) did not impair differentiation of DCPs to cDC1 (FIGS. 4B-4C).

To study the effects of the abovementioned ILs on the polarization of T cells, OVA-loaded cDC1-like cells were then cocultured with OVA-specific CD8⁺ (OT-I) or CD4⁺ (OT-II) T cells, in the presence of the selected ILs (3 independent replicates per condition). Briefly, 50,000 cDC1 were seeded on U-bottom 96 wells with 0.5 mg OVA protein (vac-stova) or the OVA peptide SIINFEKL for 4 h. The cDC1 were washed and then co-cultured with OT-I cells for 3 days or OT-II cells for 5 days. T cell activation was measured by intracellular staining with antibodies against interferon-gamma (IFNγ) using BD Golgi Stop kit (Cat No. 554715), according to the manufacturer's protocol. Results indicated that IL-12 could induce robust IFNγ production by both OT-I and OT-II T cells (FIGS. 4D-4F).

Statistical analysis: d-e) One-way ANOVA with Tukey's multiple comparison test. All samples are compared to “untreated” sample. P values are coded as *: P<0.05; **: P<0.01; ***: P<0.001; and ****: P<0.0001.

This example thus demonstrates that IL-12 does not impair differentiation of DCPs into cDC1 and enhances the co-stimulatory capacity of the cDC1 progeny in vitro.

### Example 5: DCPs Engineered to Express IL-12 and IL-2 Differentiate into cDCs in Mice

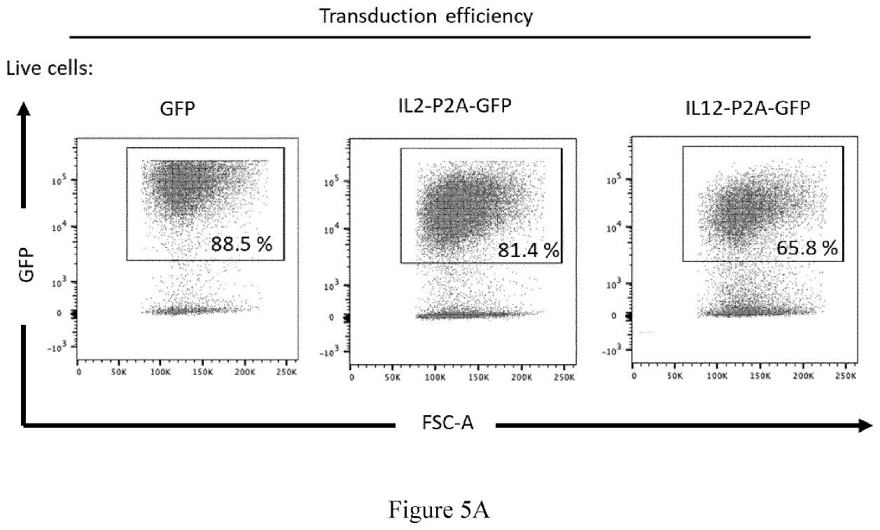

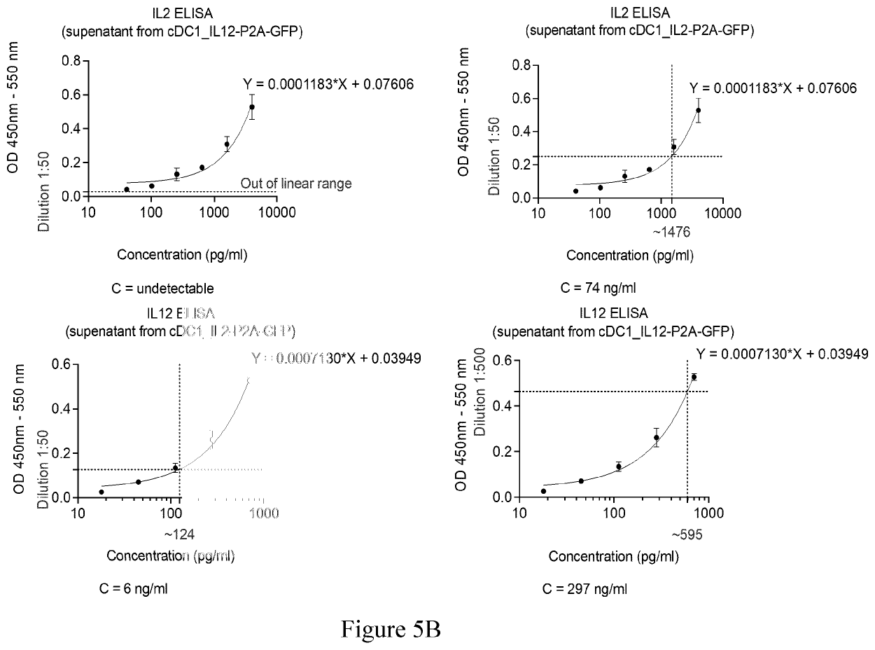

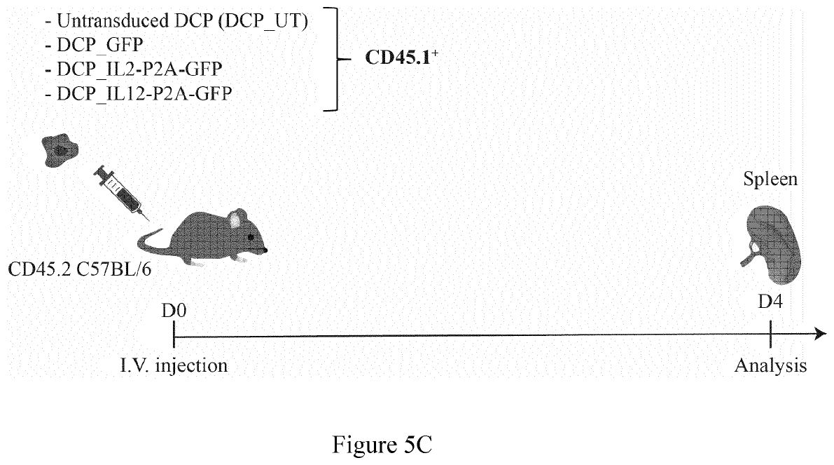

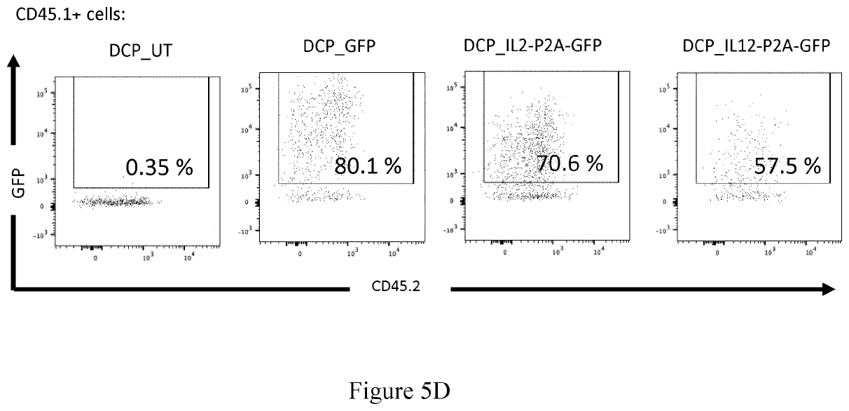

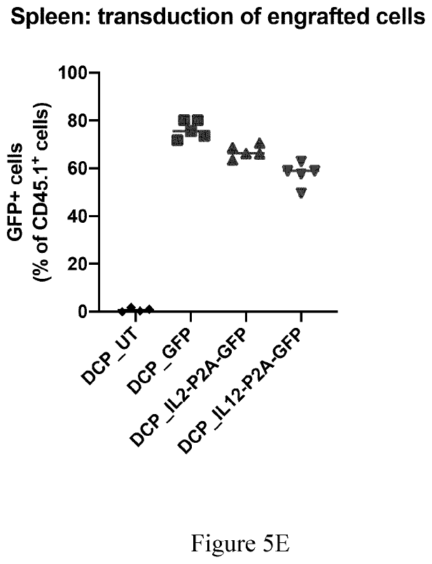

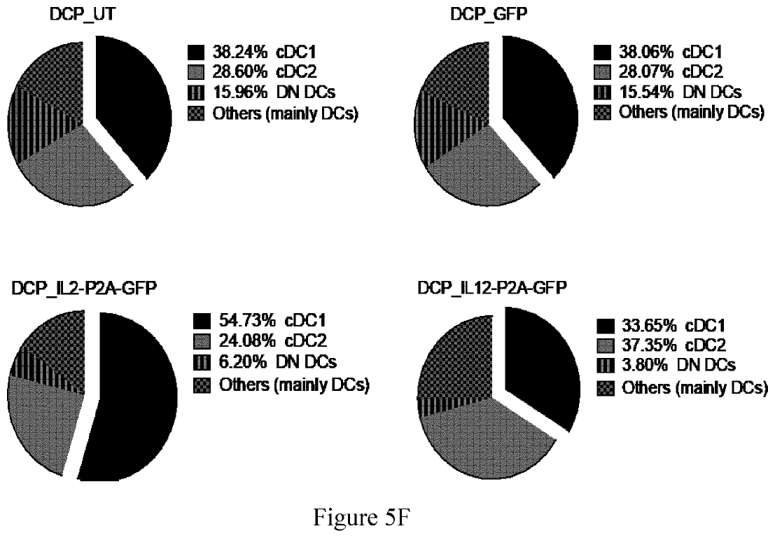

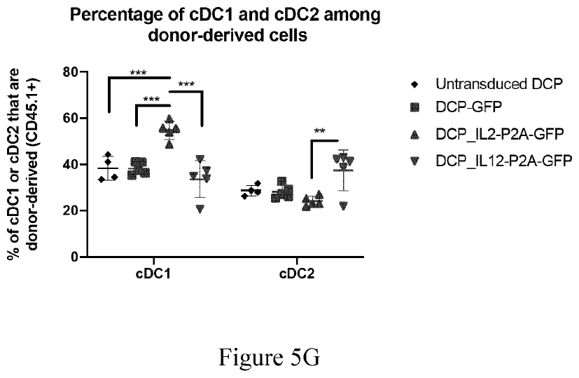

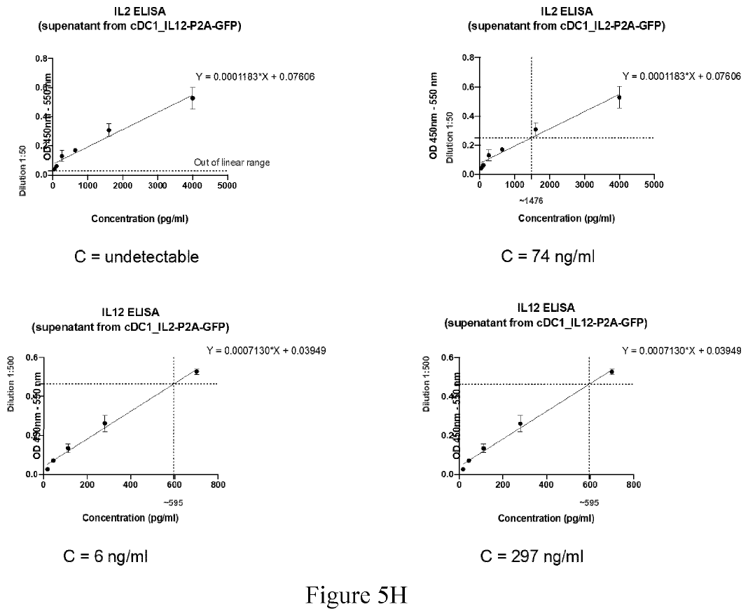

As shown in example 4, exogenous IL-12 and IL-2 did not interfere with the differentiation of DCPs into cDC1 in vitro; moreover, IL-12 enhanced the co-stimulatory capacity of cDC1-like cells. Accordingly, transgenic expression of either IL-12 or IL-2 was investigated for the ability to support DCP differentiation into cDC1 and cDC2 in vivo. To this aim, lentiviral vectors (LVs) were generated expressing either IL-12 or IL-2 together with the marker gene, green fluorescent protein (GFP, SEQ ID NO; 1), termed IL12-P2A-GFP (SEQ ID NO: 5) and IL2-P2A-GFP (SEQ ID NO: 3) LV, respectively. Monocistronic LV were also generated only expressing GFP, termed GFP LV Flow cytometry analysis of mouse BM cells transduced in HSPC medium with LVs at a multiplicity of infection (MOI) of 350 and then differentiated in cDC1 medium showed robust expression of GFP at 5 days post-transduction (FIG. 5A; differences in GFP expression between LVs can be attributed to their different infectivity, which is influenced by the size of the transgenes), a time point when transduced cells mainly comprise DCPs. Furthermore, cells transduced with IL12-P2A-GFP and IL2-P2A-GFP LVs secreted IL-12 and IL-2, respectively, in the cell culture medium, as assessed by ELISA at 2 weeks post-transduction (FIGS. 5B and 5H; “C” indicates concentration), a time point when transduced cells mainly comprise cDC1. Thus, LVs enable robust transduction of DC precursors and sustained expression of either IL-12 or IL-2 in the cDC progeny.

The in vivo differentiation potential of untransduced DCPs (UT) and DCPs transduced with_IL12-P2A-GFP, and IL2-P2A-GFP or GFP LVs were next investigated. DCPs were generated from the BM of CD45.1 mice as described in Example 1. Enriched DCPs were transferred to cDC1 medium at a concentration of 1.5×10⁶ cells/ml and concurrently transduced with LVs at the MOI of 350. The transduced DCPs were harvested 12 h after transduction and infused into recipient mice (5 mice per condition). Each mouse received 2×10⁶ DCPs; the recipient mice were sacrificed 4 days after the infusion of DCPs and splenocytes (isolated by smashing the spleen, as explained in Example 2) were analyzed for the presence of donor-derived (CD45.1⁺) cells (FIG. 5C).

Flow cytometry analysis of splenocytes revealed that the majority of CD45.1⁺ cells expressed GFP (FIGS. 5D-5E), indicative of efficient LV transduction, transgene expression, and lack of counterselection of transduced cells in vivo (differences in GFP expression between LVs can be attributed to their different infectivity, which is influenced by the size of the transgenes). Importantly, transduction of DCPs with IL-12 or IL-2 did not prevent their differentiation into cDC1 and cDC2 (FIGS. 5F-5G), consistent with the in vitro studies shown in Example 4. However, whereas IL-2 slightly enhanced cDC1 production, IL-12 slightly enhanced cDC2 production.

Statistical analysis: f) One-way ANOVA with Tukey's multiple comparison test. P values are coded as *: P<0.05; **: P<0.01; ***: P<0.001; and ****: P<0.0001.

This example thus demonstrates that transgenic expression of IL-12 does not impair differentiation of DCPs into cDC1 in vivo.

### Example 6: DCPs Engineered to Express IL-12 and a Tumor Antigen Boost the Expansion of Tumor Antigen-Specific T Cells in Mice

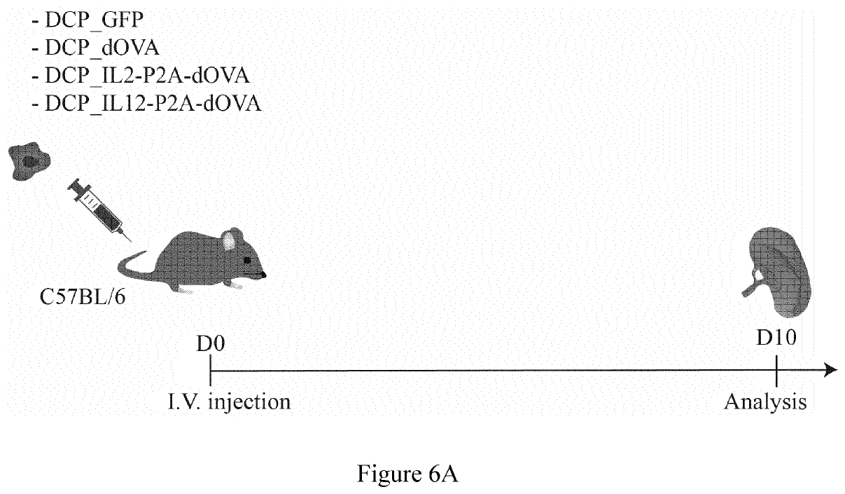

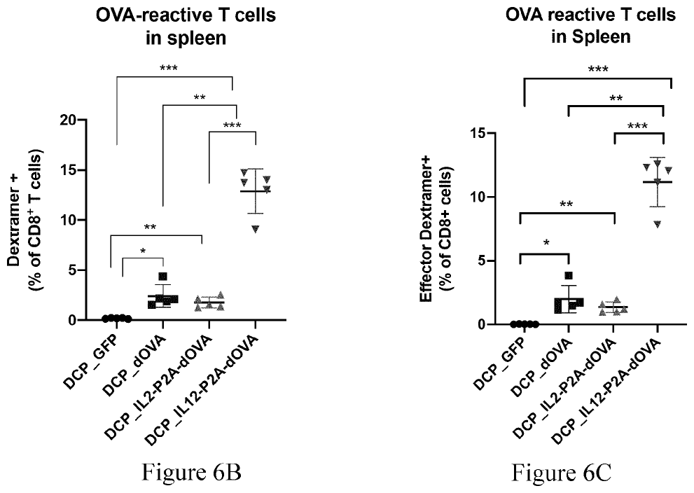

As shown in Examples 4 and 5, IL-2 and IL-12 do not prevent the differentiation of DCPs into cDC1 in vitro and in vivo. In vitro data in Example 4 also demonstrated that IL-12 can activate both CD4⁺ and CD8⁺ T cells. To investigate whether IL-12 and IL-2 can activate antigen-specific T cells in vivo, either cytokine were co-expressed together with a truncated intracellular OVA (dOVA, SEQ ID NO: 7) sequence in DCPs by LV transduction. For transduction, enriched DCPs were transferred to cDC1 medium at a concentration of 1.5×10⁶ cells/ml and concurrently transduced with LVs coding for GFP, dOVA, IL2-P2A-dOVA (SEQ ID NO: 9) or IL12-P2A-dOVA (SEQ ID NO: 11), at MOI of 350. The cells were harvested 12 h after transduction and 0.7×10⁶ transduced DCPs were injected into tumor-free mice. FIG. 6A shows the schematic of the experiment.

Transduced DCPs were infused in tumor-free mice (5 mice per condition). The recipient mice were sacrificed 10 days after vaccination and splenocytes (isolated by smashing the spleen as explained in Example 2) were stained with OVA₂₅₇₋₂₆₄-bound dextramer according to manufacturer's protocol (Immunex) to identify OVA-reactive T cells. Flow cytometry data demonstrated that expression of IL-12 could dramatically boost OVA-reactive T cells, with ˜12.8% of all T cells in the spleen being OVA-reactive following a single DCP vaccination (FIG. 6B). Of note, DCPs expressing dOVA alone and DCPs expressing dOVA together with IL-2 induced a much weaker OVA-specific response. To study the phenotype of the OVA-reactive T cells in vaccinated mice, splenocytes were also stained with anti-CD44 and anti-CD62L antibodies. Flow cytometry analysis revealed that IL12 strongly enhanced the expansion of effector (CD44⁺ CD62L-neg) OVA-reactive T cells (FIG. 6C).

Statistical analysis: b-c) One-way ANOVA with Tukey's multiple comparison test. P values are coded as *: P<0.05; **: P<0.01; ***: P<0.001; and ****: P<0.0001.

This example thus demonstrates that DCPs co-expressing IL-12 and a tumor antigen boost tumor antigen-specific T cells in vivo.

### Example 7: DCPs Engineered to Express IL-12 Together with a Tumor Antigen Block Tumor Initiation in Mice

A prophylactic vaccination study was performed to study the anti-tumor potential of DCP_IL2-P2A-dOVA and DCP_IL12-P2A-dOVA cells.

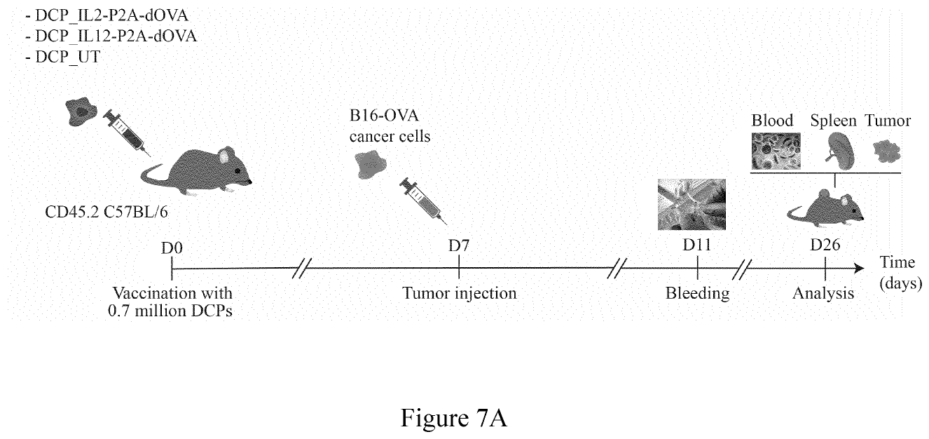

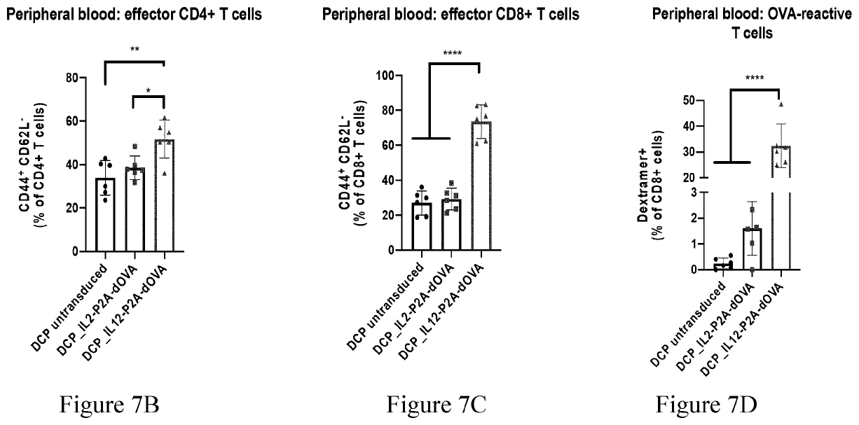

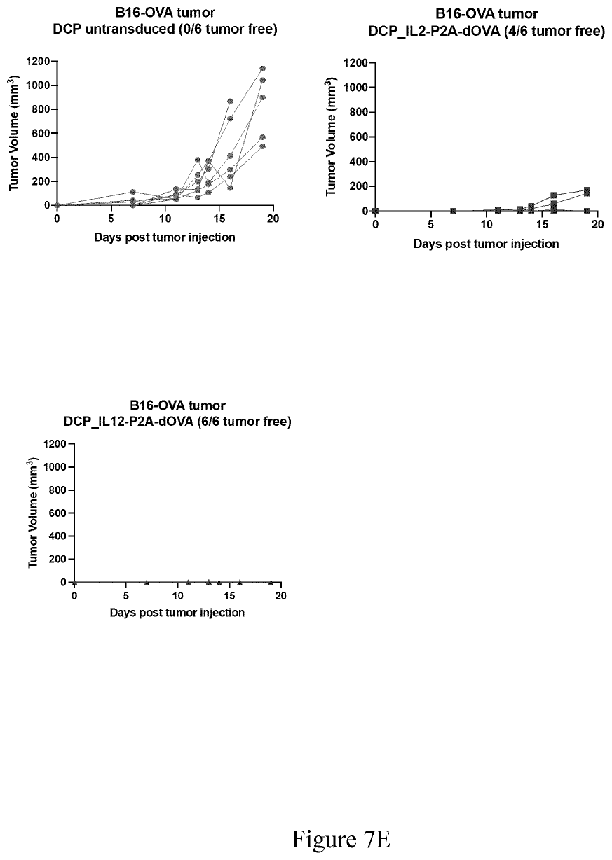

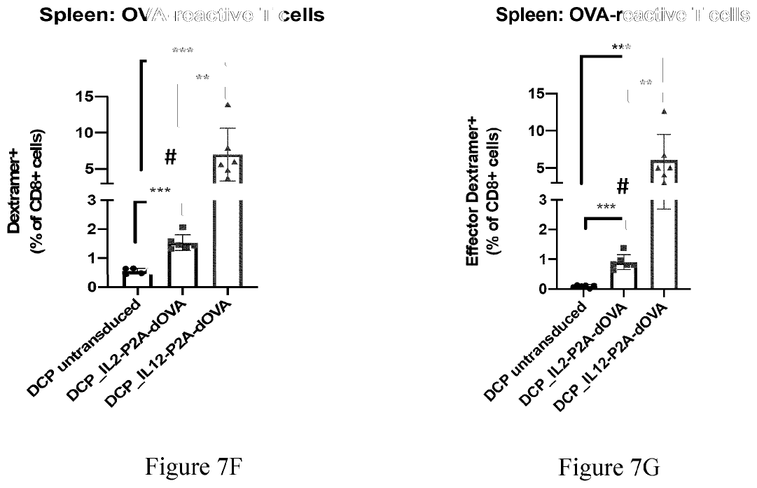

Transduced DCPs were produced as described in Example 6 above. Both transduced and untransduced DCPs were then intravenously injected in tumor free mice (0.7×10⁶ cells per mouse; 6 mice per condition). The mice were then inoculated subcutaneously with B16-OVA cancer cells (5×10⁵ cells) one week after vaccination (FIG. 7A).

A blood sample was taken from the mice 4 days after tumor challenge and circulating T cells were analyzed by flow cytometry. Co-expression of IL-12 and dOVA increased effector T cells (CD44+CD62L-neg) within both CD4⁺ and CD8⁺ T cells (FIGS. 7B-7C). Staining of T cells with OVA₂₅₇₋₂₆₄-bound dextramer indicated that almost a third of all circulating CD8⁺ T cells were OVA-reactive following vaccination with DCP_IL12-P2A-dOVA (FIG. 7D). Conversely, IL-2 stimulated weaker immune responses against OVA.

Vaccination of the mice with DCP_IL12-P2A-dOVA cells fully prevented tumor growth (FIG. 7E). Conversely, DCP_IL2-P2A-dOVA cells did not fully prevent tumor growth. The spleen of the mice was isolated and purified 26 days after vaccination (19 days post-tumor challenge). Flow cytometry analysis indicated robust expansion of OVA-reactive effector T cells (FIGS. 7F-7G) in mice vaccinated with DCP_IL12-P2A-dOVA cells, consistent with results obtained in tumor-free mice (see Example 6 above). Conversely, IL-2 stimulated weaker immune responses against OVA.

Statistical analysis: B-D, F-G) One-way ANOVA with Tukey's multiple comparison test. The # symbol in f-g indicates comparison between two groups of interest using unpaired Student's t test. P values are coded as *: P<0.05; **: P<0.01; ***: P<0.001; and ****: P<0.0001.

This example thus demonstrates that DCPs co-expressing IL-12 and a tumor antigen boost tumor antigen-specific T cells and protect mice from tumor challenge.

### Example 8: DCPs Engineered to Express IL-12 Together with a Tumor Antigen Inhibit Tumor Growth

Owing to their superior efficacy, DCP cells expressing IL12 were prepared and tested as a DCP vaccination in a therapeutic setting involving vaccination of tumor-bearing mice.

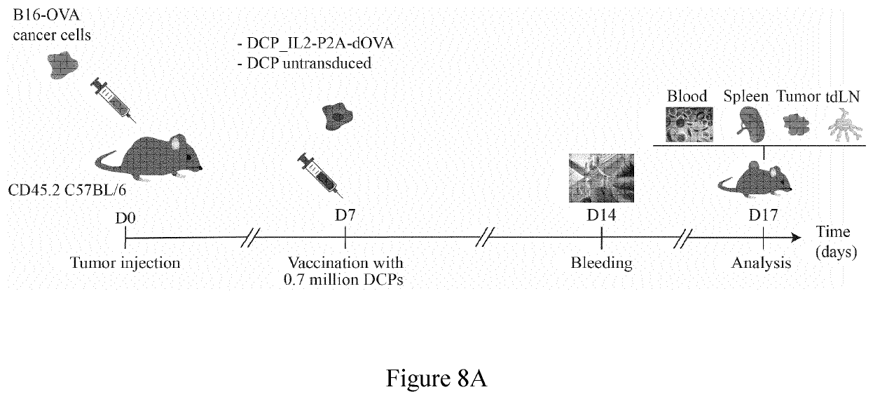

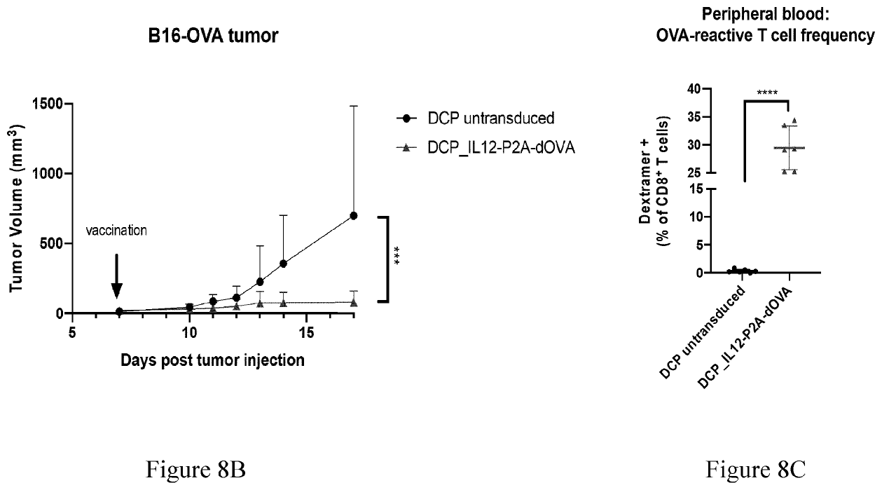

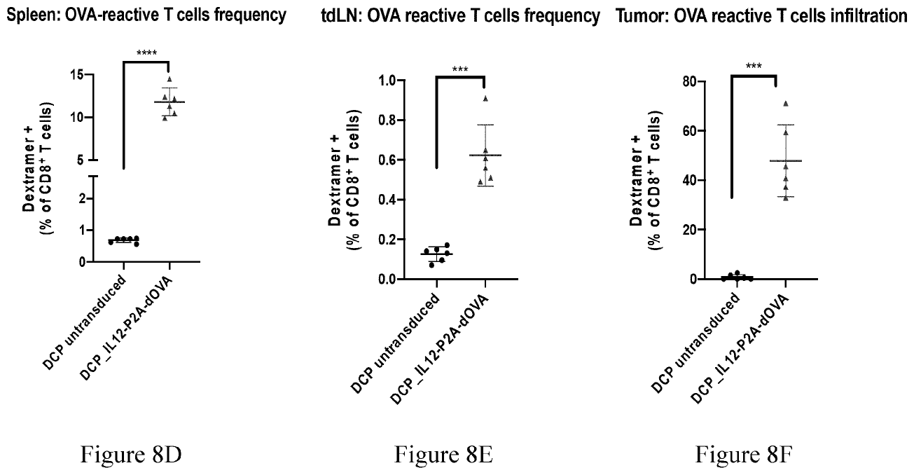

The mice were inoculated subcutaneously with B16-OVA cancer cells (5×10⁵ cells) and intravenously infused with transduced or untransduced DCPs (0.7×10⁶ cells; 6 mice per condition) one week post-tumor challenge (FIG. 8A).

DCP_IL12-P2A-dOVA cells markedly inhibited tumor growth (FIG. 8B). Furthermore, DCP_IL12-P2A-dOVA cells boosted OVA-reactive T cells in blood, spleen, tumor-draining lymph node (tdLN) and tumor of vaccinated mice (FIGS. 8C-8F), as shown by staining of immune cells with OVA₂₅₇₋₂₆₄-bound dextramer. Remarkably, about half of all CD8⁺ T cells were, on average, OVA-reactive in the tumors of mice vaccinated with DCP_IL12-P2A-dOVA cells, which is consistent with the magnitude of anti-tumoral response observed.

Statistical analysis: b) Two-way ANOVA with Sidak multiple comparison test. c-f) unpaired Student's t test. P values are coded as *: P<0.05; **: P<0.01; ***: P<0.001; and ****: P<0.0001.

This example thus demonstrates that DCPs co-expressing IL-12 and a tumor antigen boost tumor antigen-specific T cells and inhibit tumor growth.

### Example 9: DCPs Engineered to Express IL-12 Together with EVIR Enable Tumor-Antigen Agnostic Vaccination and Inhibit Tumor Growth

The studies shown in Examples 7-8 above used DCPs transduced with IL-12 together with a defined surrogate tumor antigen (dOVA) to vaccinate mice with OVA-expressing tumors. The ability of DCPs transduced with IL-12 to elicit anti-tumor immunity in a tumor antigen agnostic manner, i.e., without enforcing the expression of any tumor antigen by the DCPs, was next investigated.

Extracellular vesicle (EV)-internalizing receptor (EVIR) was used with an extracellular scFv domain directed against GD2, a disialogangloside expressed on the plasma membrane of both mouse and human melanomas and their secreted EVs. Unlike conventional DC vaccination against defined tumor antigen(s), which involves in vitro antigen loading of the DCs and elicits a T cell response only against the targeted (loaded) antigen(s), vaccination with EVIR-expressing DCs can elicit a T cells response potentially targeting any EV-associated tumor antigen. Notably, vaccination with EVIR-expressing DCs does not require apriori knowledge of tumor antigens, with the exception of the molecule (e.g., GD2) used to capture tumor EVs in vivo.

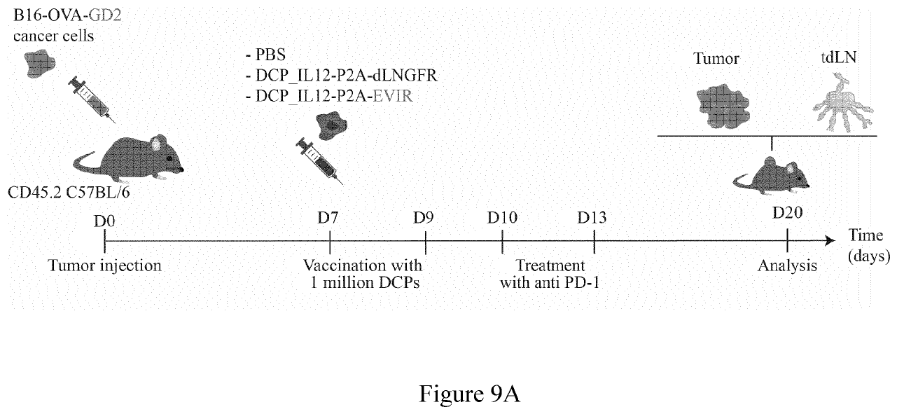

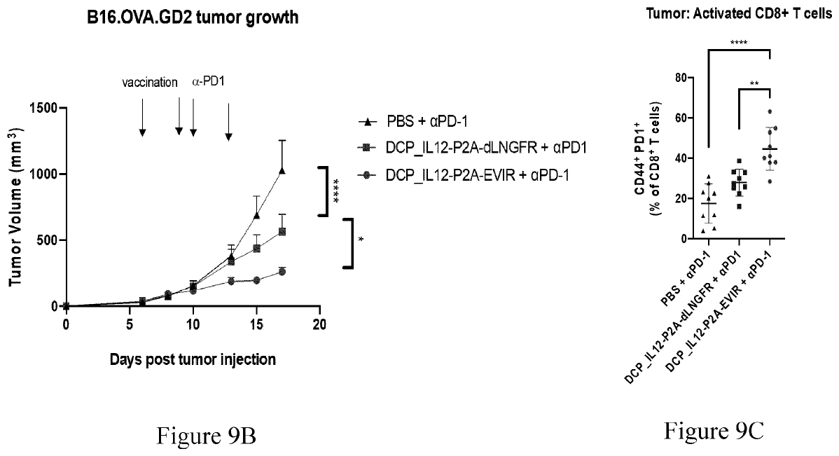

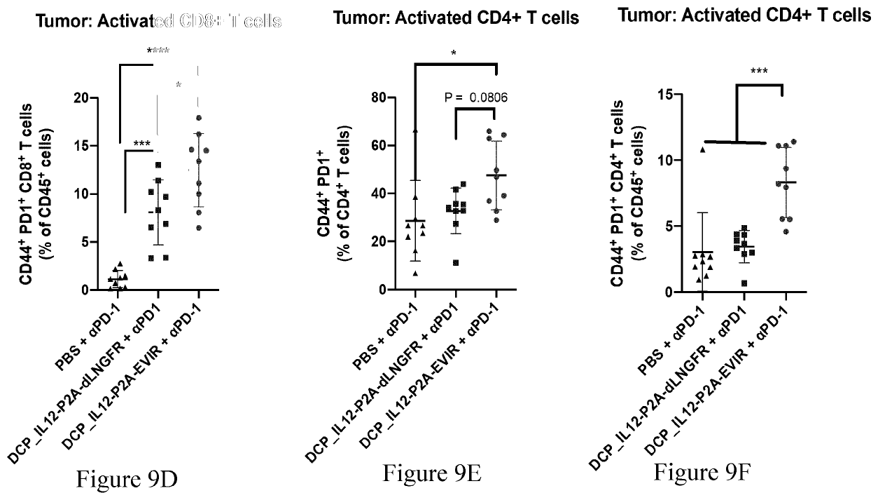

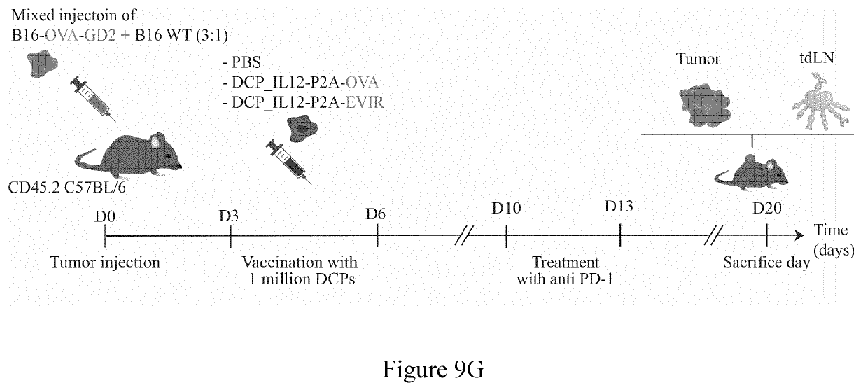

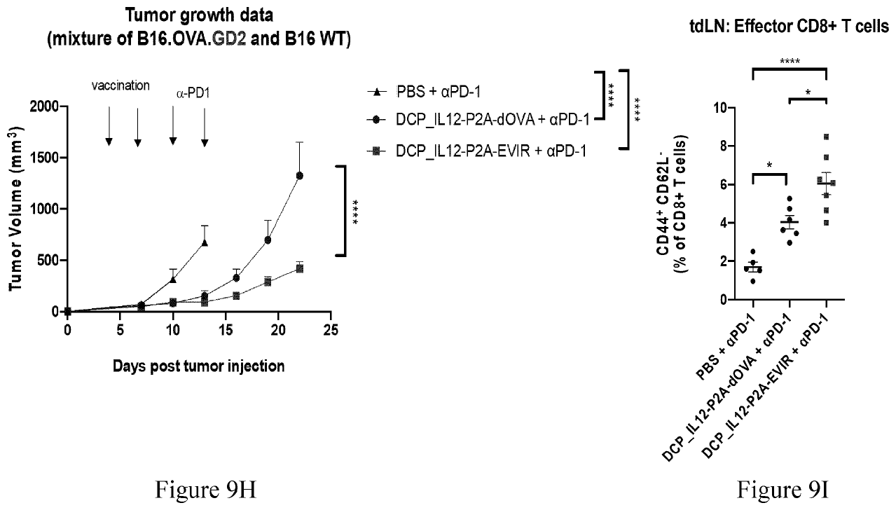

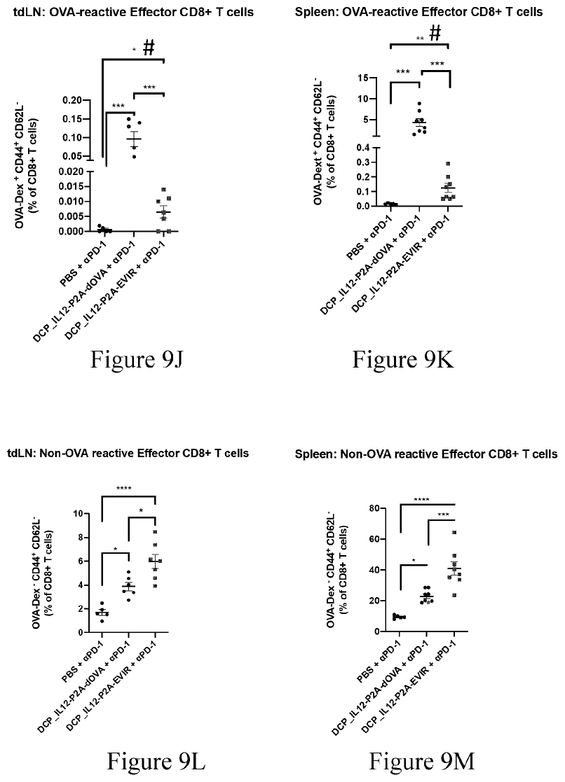

Bicistronic IL12-P2A-EVIR (SEQ ID NO: 17) LV was generated to express both IL-12 and the EVIR in the DCPs. IL12-P2A-dLNGFR (SEQ ID NO: 15) LV was also generated, which expresses IL-12 together with a control non-signaling receptor (truncated low-affinity nerve growth factor receptor, dLNGFR, SEQ ID NO: 13) lacking the extracellular scFv domain of the EVIR. Vaccination studies were then performed in tumor-bearing mice (FIG. 9A). For the generation and transduction of DCPs (DCP_IL12-P2A-EVIR or DCP_IL12-P2A-dLNGFR), the same procedure as shown in Examples 5-8 above were used. The mice (8 mice per condition) were inoculated subcutaneously with B16-OVA-GD2 cancer cells (5×10⁵ cells) and intravenously infused with transduced DCPs (1×10⁶ cells on day 7 and 9 post-tumor challenge) or PBS (as mock treatment). The mice were also treated with an anti-PD1 antibody to unleash the effector functions of T cells at the time points indicated in FIG. 9A.

Vaccination with DCP_IL12-P2A-EVIR cells led to significantly better tumor control than vaccination with DCP_IL12-P2A-dLNGFR cells lacking the functional EVIR (FIG. 9B); of note, this result was achieved without OVA vaccination. The mice were then sacrificed, and the tumors analyzed by flow cytometry. Flow cytometry analysis revealed that vaccination with DCP_IL12-P2A-EVIR cells led to increased tumor infiltration by activated CD4⁺ and CD8⁺ T cells in the tumor microenvironment, as compared to vaccination with DCP_IL12-P2A-dLNGFR lacking the functional EVIR (FIGS. 9C-9F).

The mice were inoculated subcutaneously with a mixture of B16 (1×10⁵ cells) and B16-OVA-GD2 (3×10⁵ cells) melanoma cells and vaccinated with DCP_IL12-P2A-EVIR (8 mice) or DCP_IL12-P2A-dOVA cells (1×10⁶ cells on day 3 and 6 post-tumor challenge; 9 mice), or mock-treated (PBS; 5 mice). All mice were also treated with an anti-PD1 antibody to unleash the effector functions of T cells (FIG. 9G).

Vaccination with DCP_IL12-P2A-EVIR cells produced greater tumor control than vaccination with DCP_IL12-P2A-dOVA cells (FIG. 9H), showing that the EVIR could also elicit anti-tumoral immunity against B16 melanoma cells lacking OVA or GD2. Accordingly, DCP_IL12-P2A-EVIR vaccination elicited increased effector T cells in the tdLNs (FIG. 9I). Moreover, DCP_IL12-P2A-EVIR vaccination induced OVA-specific T cells in tdLN and spleen (see comparison with PBS-treated mice), although their frequency was expectedly lower than in mice vaccinated with DCP_IL12-P2A-dOVA cells (FIGS. 9J-9K). Of note, analysis of tdLN and spleen of mice vaccinated with DCP_IL12-P2A-EVIR cells demonstrated expansion of non-OVA-reactive effector T cells, compared with mice vaccinated with DCP_IL12-P2A-dOVA cells (FIGS. 9L-9M), which may explain the more marked tumor control against B16 tumors containing OVA-negative clones.

Statistical analysis: b, h) Two-way ANOVA with Tukey's multiple comparison test. c-f, i-m) One-way ANOVA with Tukey's multiple comparison test. The # symbol in j-k indicates comparison between two groups of interest by unpaired Student t test. P values are coded as *: P<0.05; **: P<0.01; ***: P<0.001; and ****: P<0.0001.

This example thus shows that DCPs co-expressing IL-12 and EVIR inhibit tumor growth in a tumor-antigen agnostic fashion and are superior to vaccination against a defined tumor antigen.

### Example 10: DCPs Engineered to Express IL-12 and FLT3L Enable Highly Effective Tumor-Antigen Agnostic Vaccination

DCPs either expressing IL-12 or FLT3L were generated by LV transduction and vaccination studies were performed in mice by mixing the two DCP populations. In order to track transduced DCPs and their progeny, IL-12 was coupled to dLNGFR, whereas FLT3L was coupled to GFP (both dLNGFR and GFP are neutral marker proteins).

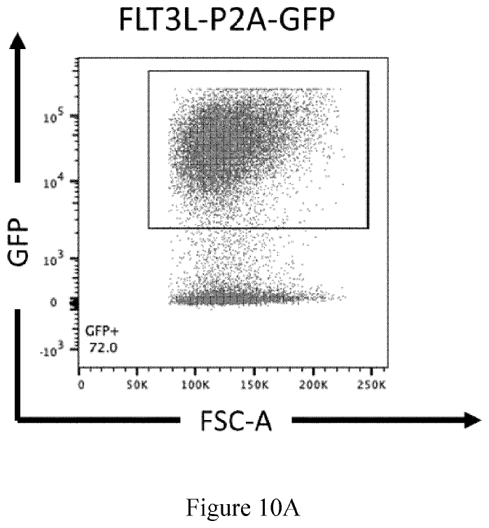

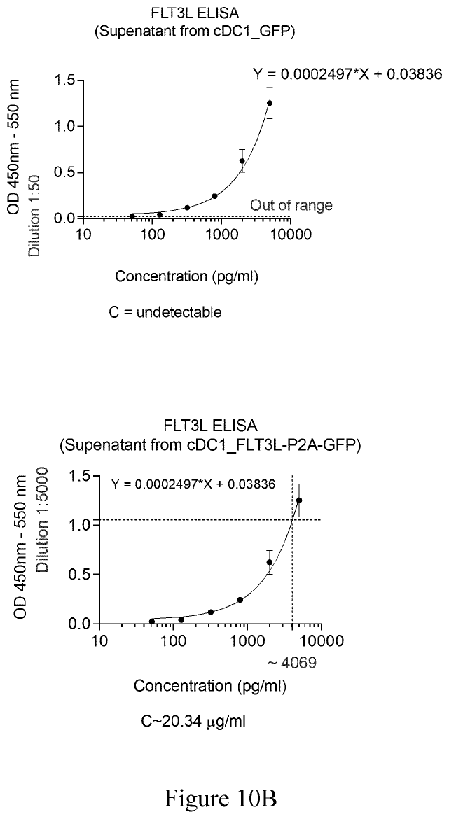

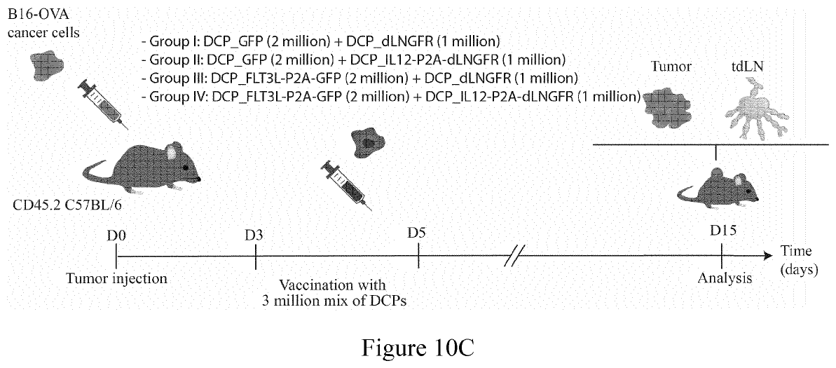

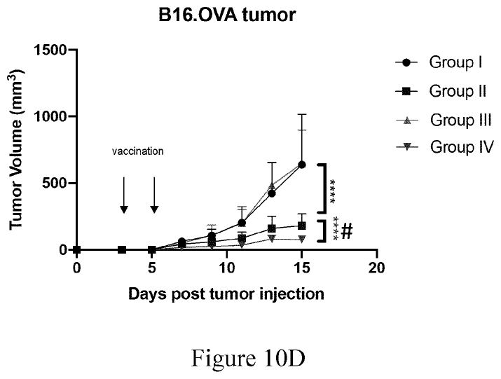

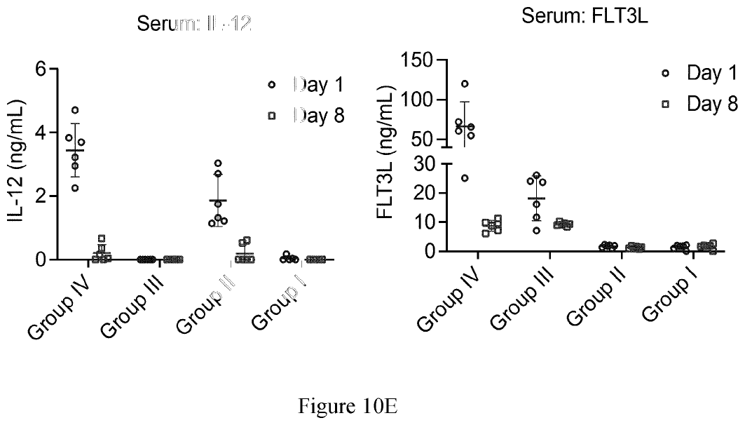

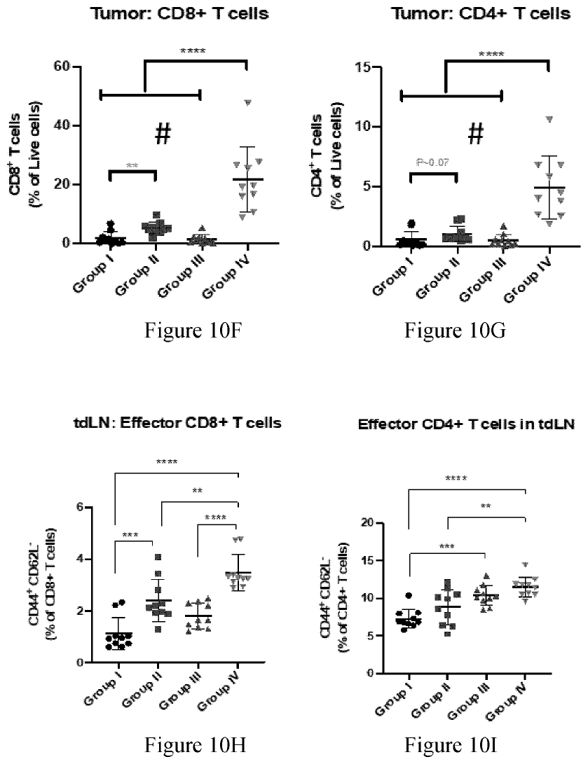

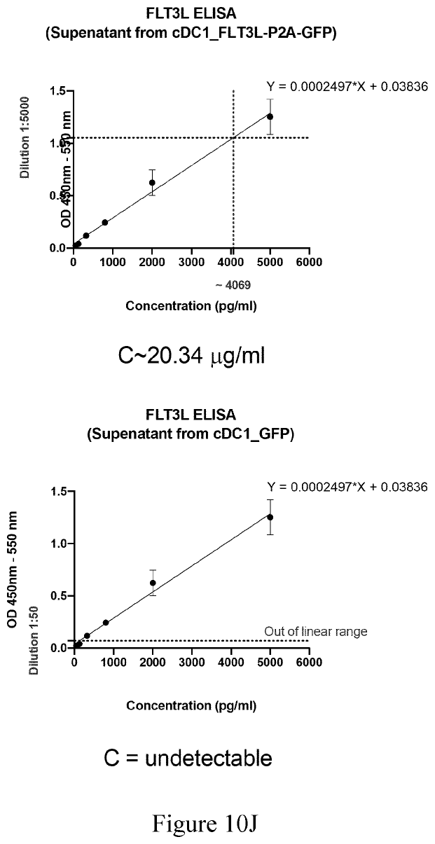

To express FLT3L in DCPs, a FLT3L-P2A-GFP (SEQ ID NO: 19) LV was constructed and was validated as performed for the IL-12 and IL-2 encoding LVs (see Example 5a-b above). Briefly, flow cytometry analysis of mouse BM cells transduced in HSPC medium with LVs at a multiplicity of infection (MOI) of 350 and then differentiated without exogenous FLT3L showed robust expression of GFP at 5 days post-transduction (FIG. 10A), a time point when transduced cells mainly comprise DCPs. Furthermore, cells transduced with FLT3L-P2A-GFP LV secreted FLT3L in the cell culture medium, as assessed by ELISA at 2 weeks post-transduction (FIGS. 10B and 10J), a time point when transduced cells mainly comprise cDC1. Thus, LV enable robust transduction of DC precursors and sustained expression of FLT3L in the cDC progeny.

Vaccination studies were then performed in tumor-bearing mice (FIG. 10C). The mice (10 mice per condition) were inoculated subcutaneously with B16-OVA melanoma cells (5×10⁵ cells) and infused intravenously twice (on day 3 and 5 post-tumor challenge) with a mixture of 1×10⁶ DCP_IL12-P2A-dLNGFR cells and 2×10⁶ DCP_FLT3L-P2A-GFP cells. To study DCPs expressing only one cytokine (i.e., either IL-12 or FLT3L), the cells were mixed with the appropriate number of control DCPs either expressing GFP or dLNGFR. Control mice received DCPs only expressing GFP and dLNGFR.

As shown in FIG. 10D, the combination of DCP_FLT3L-P2A-GFP and DCP_IL12-P2A-LNGFR cells achieved better tumor control than DCP_IL12-P2A-dLNGFR (plus control DCP_GFP) cells. Of note, DCP_FLT3L-P2A-GFP (plus control DCP_dLNGFR) cells was not effective, indicating synergistic activity of the combination of DCP_FLT3L-P2A-GFP and DCP_IL12-P2A-dLNGFR cells. To measure systemic levels of transgenic cytokine in vaccinated mice, serum samples were collected from the tail vein at day 1 and 8 after the last DCP infusion. ELISA of serum IL-12 (BD 555256, BD Bioscieces) and FLT3L (EMFLT3L, Invitrogen) showed an early and transient increase of transgenic cytokines in mice that received DCP_FLT3L-P2A-GFP and DCP_IL12-P2A-dLNGFR, which was followed by rapid decrease to quasi-baseline levels on day 8 post-vaccination (FIG. 10E). Interestingly, flow cytometry analysis of intra-tumoral T cells revealed greater CD8⁺ and CD4⁺ T cell infiltrates after the combined vaccination (FIGS. 10F-10G). Moreover, the combination of DCP_FLT3L-P2A-GFP and DCP_IL12-P2A-LNGFR induced expansion of effector CD8⁺ T cells and CD4⁺ T cells in tdLNs, compared to all other vaccination groups (FIGS. 1011-10I).

Statistical analysis: d) Two-way ANOVA with Tukey's multiple comparison test. e-h) One-way ANOVA with Tukey's multiple comparison test. The # symbol in d indicates comparison between two groups of interest by two-way ANOVA with Sidak multiple comparison test. The # symbol in e-f indicates comparison between two groups of interest by unpaired Student t test. P values are coded as *: P<0.05; **: P<0.01; ***: P<0.001; and ****: P<0.0001.

This example thus demonstrates that DCPs expressing IL-12 and FL3TL inhibit tumor growth in a tumor-antigen agnostic fashion.

### Example 11: DCPs Engineered to Express IL-12 and FLT3L are Superior to moDCs and cDC1

The ability of transgenically expressed FLT3L and IL12 to endow other populations of DCs, namely moDCs or mature cDC1 (see Example 2 above), with the ability to expand T cells and control tumor growth was next investigated.

DCPs, moDCs and cDC1 were generated from the BM of CD45.1 mice as explained in Example 2 and transduced with LVs encoding FLT3L, IL-12 and/or marker genes, as explained in Example 10 above. Briefly, DCPs and moDCs were transduced at the end of day 8 of the differentiation protocol, whereas cDC1 were transduced at the end of day 16 of the differentiation protocol.

moDCs were seeded at a concentration of 1.5×10⁶ cells/ml in moDC medium and transduced for 12-14 h with the FLT3L-P2A-GFP or IL12-P2A-dLNGFR LVs at MOI of 100. cDC1 and DCPs were seeded at a concentration of 1.5×10⁶ cell/ml in cDC1 medium and transduced for 12-14 h with the FLT3L-P2A-GFP or IL12-P2A-dLNGFR LVs at MOI of 350.

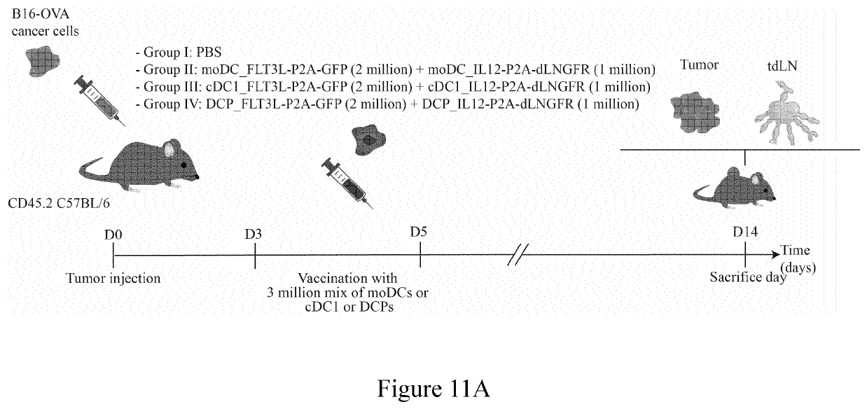

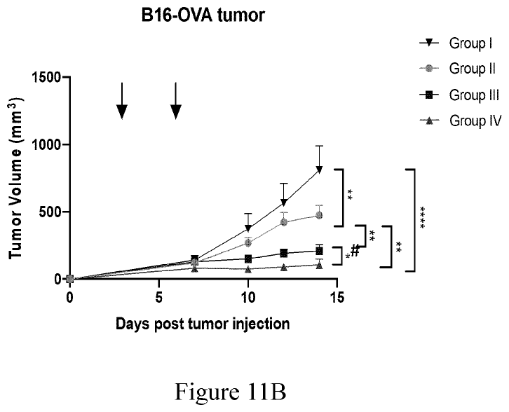

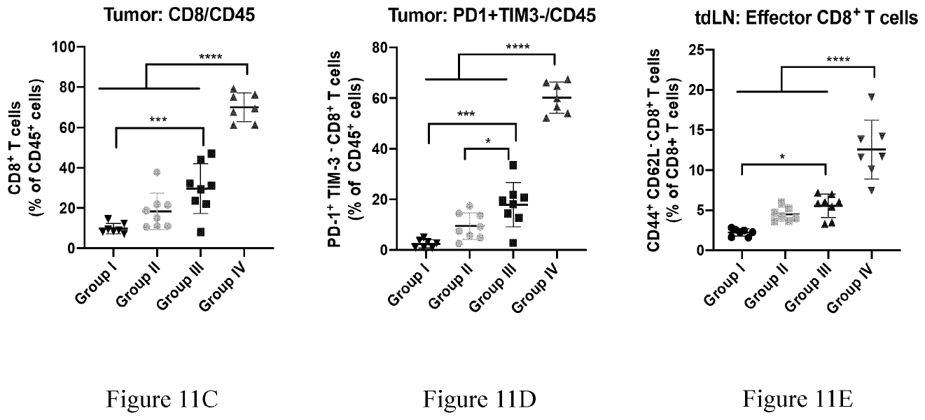

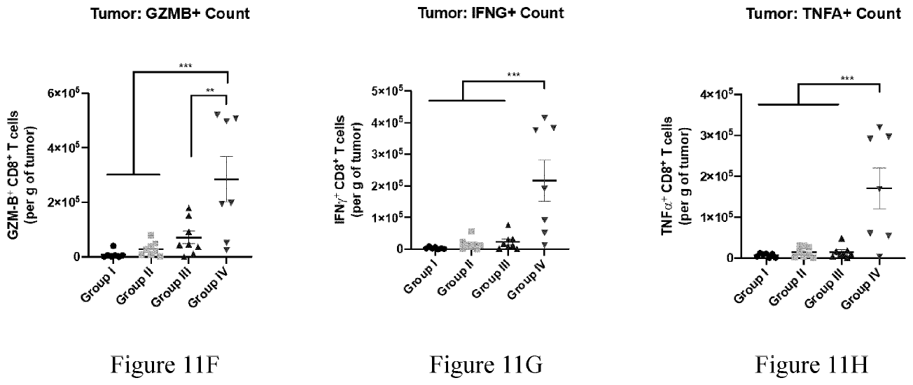

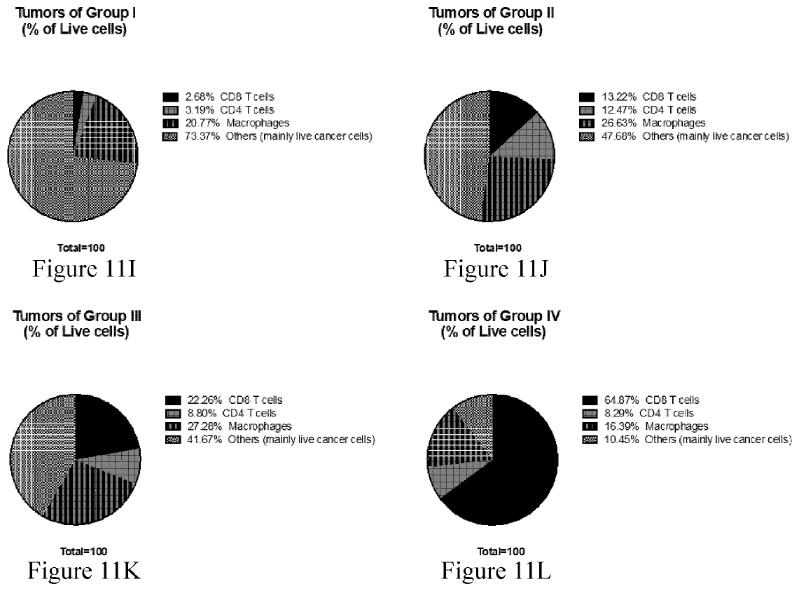

Vaccination studies were then performed in tumor-bearing mice (FIG. 11A). The mice (7-8 mice per condition) were inoculated subcutaneously with B16-OVA melanoma cells (5×10⁵) and infused intravenously twice (on day 3 and 5 post-tumor challenge) with a mixture of 1×10⁶ cells transduced with the IL12-P2A-dLNGFR LV and 2×10⁶ cells transduced with the FLT3L-P2A-GFP LV. Control mice only received PBS.

Expression of IL12-P2A-dLNGFR and FLT3L-P2A-GFP inhibited tumor growth (compared to mock-treatment) regardless of the DC type used. However, vaccination with DCPs achieved better tumor control than vaccination with either cDC1 or moDCs. (FIG. 11B). moDCs did not inhibit tumor growth as well as cDC1 or DCPs.

Flow cytometry analysis of tumors and lymph nodes of treated mice revealed that tumors treated with DCP-IL12-P2A-dLNGFR plus DCP-FLT3L-P2A-GFP cells were massively infiltrated by T cells, with the vast majority of them having an activated phenotype (FIGS. 11C-11D); the magnitude of such effects was much greater for DCPs than cDC1 and moDCs. tdLNs showed similar results (FIG. 11E). Single cell suspensions from tumors stimulated ex vivo with puromycin and ionomycin showed markedly greater production of IFNγ, GZMB and TNFA in samples from DCP-vaccinated mice, compared with the other groups (FIGS. 11F-11H). Overall, tumors of mice vaccinated with DCP-IL12-P2A-dLNGFR plus DCP-FLT3L-P2A-GFP cells displayed a broadly reprogrammed tumor microenvironment, characterized by a residual epithelial (cancer cell) component and markedly abundant T cell infiltrates (FIGS. 11I-11L).

Statistical analysis: b) Two-way ANOVA with Tukey's multiple comparison test. c-h) One-way ANOVA with Tukey's multiple comparison test. The # symbol in b indicates comparison between two groups of interest by two-way ANOVA with Sidak multiple comparison test. P values are coded as *: P<0.05; **: P<0.01; ***: P<0.001; and ****: P<0.0001.

This example thus demonstrates that DCPs expressing IL-12 and FL3TL are superior to moDCs and mature cDC1 and broadly reprogram the tumor microenvironment to a form dominated by activated T cells.

### Example 12: DCPs Engineered to Express IL-12 and FLT3L are Effective in Different Cancer Types

The main advantage of the DCP vaccination platform described herein is that this strategy does not rely on known tumor antigens. Accordingly, DCPs have the potential to function as a universal DC vaccine. In order to test the efficacy of DCP vaccination in a different tumor model, MC38 cancer cells of colorectal cancer origin were used.

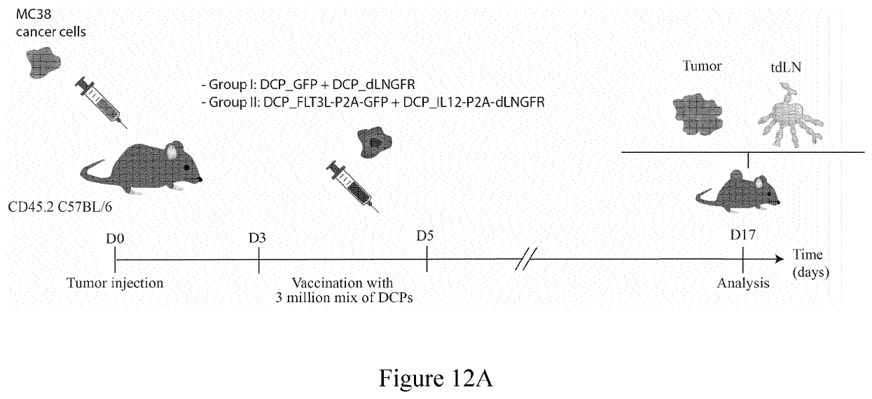

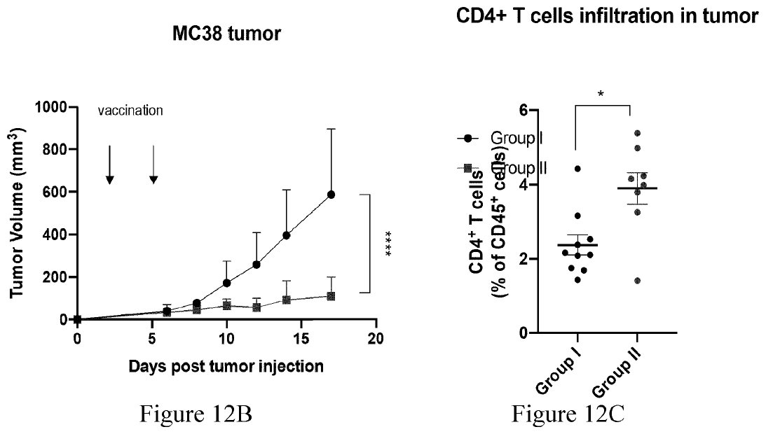

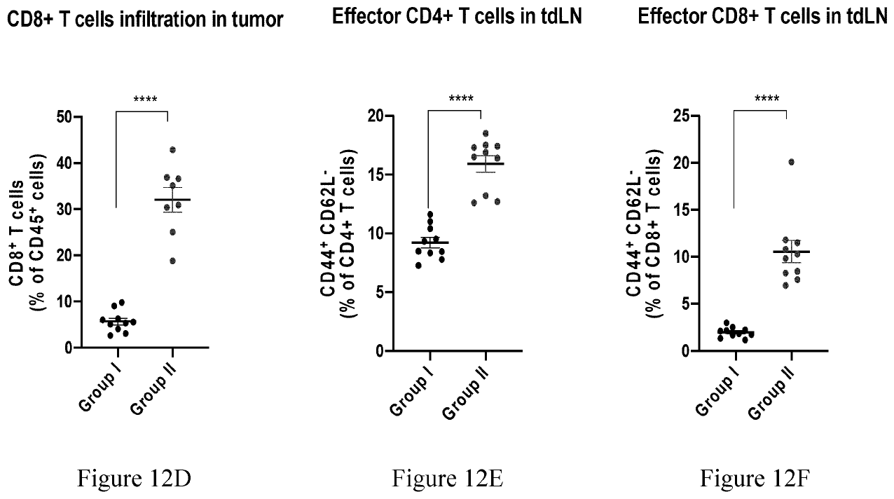

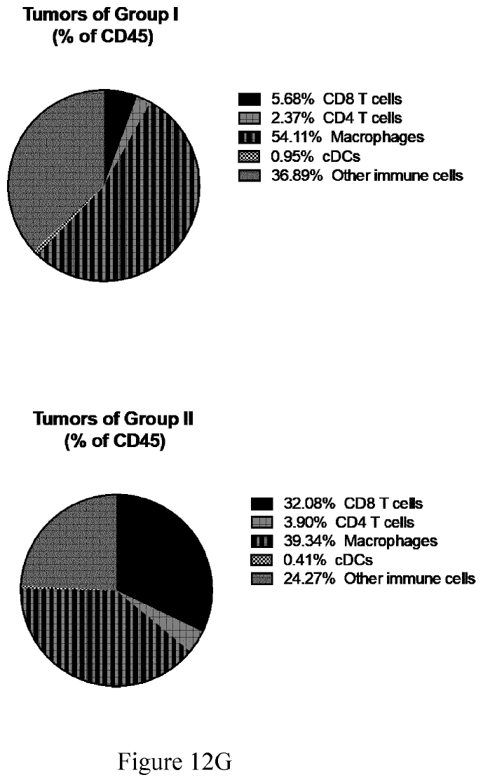

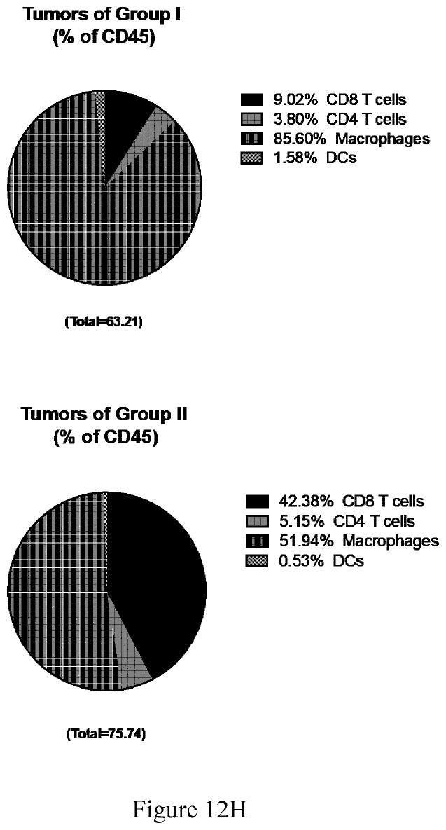

Mice (10 per condition) were inoculated subcutaneously with MC38 cancer cells (5×10⁵ cells) and were infused intravenously twice (on day 3 and 5 post-tumor challenge) with a mixture of 1×10⁶ DCP-IL12-P2A-dLNGFR cells and 2×10⁶ DCP-FLT3L-P2A-GFP cells. Control mice received equal numbers of DCPs transduced with control LVs (expressing only dLNGFR or GFP) (FIG. 12A).

DCP-IL12-P2A-dLNGFR plus DCP-FLT3L-P2A-GFP vaccination may potentially represent a highly effective tumor-agnostic or achieved robust MC38 tumor control (FIG. 12B). Consistent with results in the B16-OVA melanoma model, the engineered DCPs markedly enhanced infiltration of the tumors by CD8⁺ T cells and, to a lesser extent, CD4⁺ T cells (FIGS. 12C-12D). Moreover, the proportion of effector CD4⁺ and CD8⁺ T cells was markedly increased in the tDLNs (FIGS. 12E-12F). Overall, MC38 tumors of mice vaccinated with DCP-IL12-P2A-dLNGFR plus DCP-FLT3L-P2A-GFP cells displayed a broadly reprogrammed immune microenvironment, characterized by reduction of myeloid cells encompassing immunosuppressive macrophages and markedly enhanced CD8⁺ T cells (FIGS. 12G-12H).

Statistical analysis: b) Two-way ANOVA with Tukey's multiple comparison test. c-f) Unpaired Student t test. P values are coded as *: P<0.05; **: P<0.01; ***: P<0.001; and ****: P<0.0001.

This example thus demonstrates that DCPs expressing IL-12 and FL3TL broadly reprogram the tumor microenvironment to a form dominated by activated T cells in both melanoma and colorectal cancer models.

### Example 13: Generation of Human DCPs from CD34⁺ Progenitors

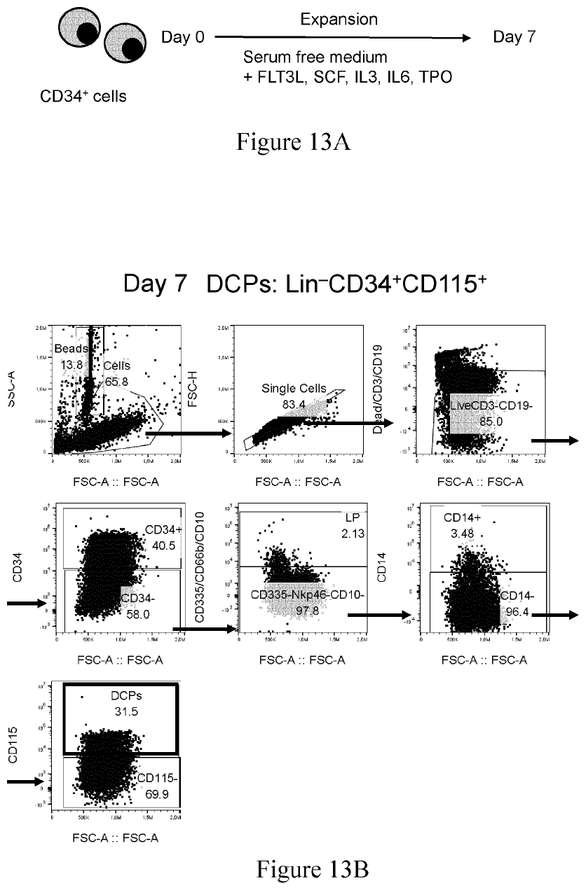

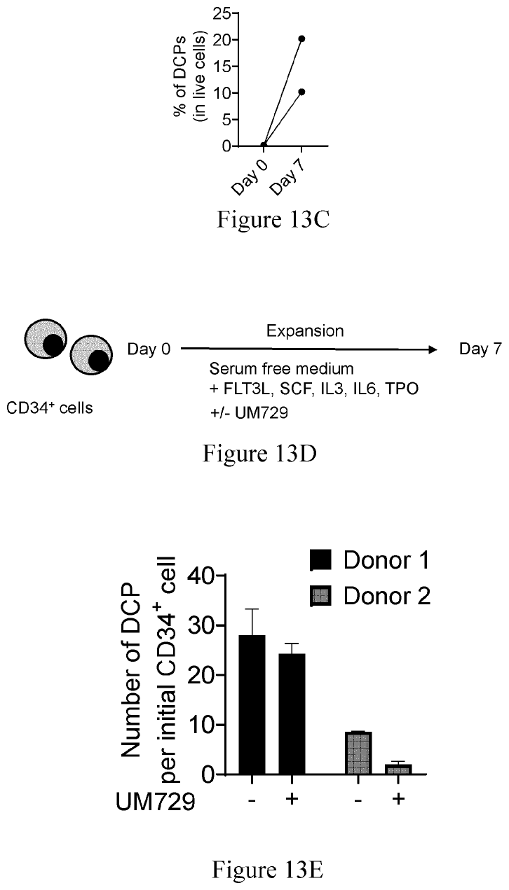

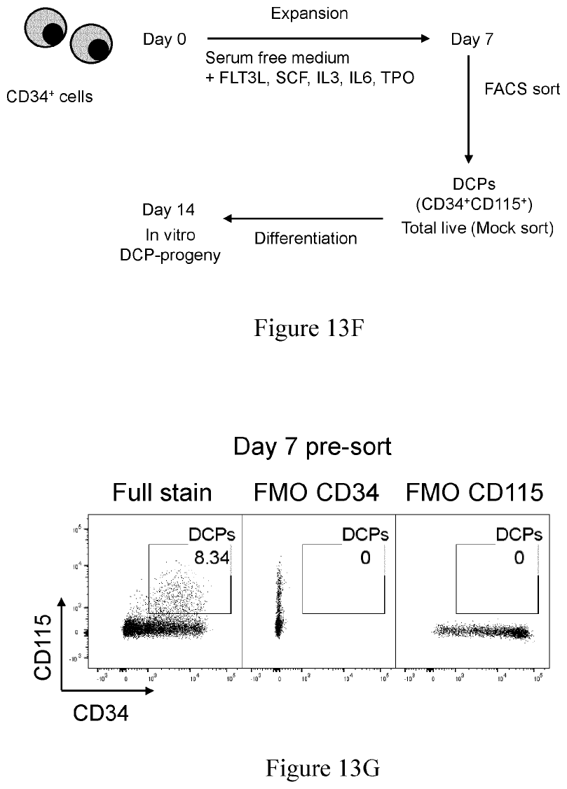

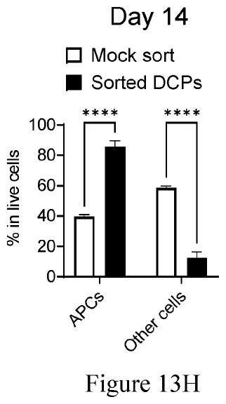

Based at least in part on the pre-clinical efficacy of cytokine-armed mouse DCPs a corresponding protocol for generation of human DCPs was developed. Cord-blood CD34⁺ progenitors were cultured at an initial concentration of 5×10⁴ cell/mL in U-bottom 96 w/plates in StemSpan SFEMII medium (Stem Cell Technologies; 09605) supplemented with StemSpan CD34⁺ Expansion Supplement (Stem Cell Technologies; 02691), which contains FLT3L, SCF, TL3, TL6 and TPO (FIG. 13A). The maximum enrichment of human DCPs, defined as CD3⁻, CD19⁻, CD335⁻, CD66b⁻, CD10⁻, CD14⁻, CD34⁺ and CD115⁺ (FIG. 13B), was obtained at day 7 (FIG. 13C). Surprisingly, addition of the stem cell expansion enhancer UM729 (Stem Cell Technologies; 72332) to the media was detrimental to the yield of human DCPs (FIG. 13D-13E). As there are no mouse models that can sustain human DCPs differentiation into bona fide cDC1 and cDC2, the ability of human DCPs to differentiate into antigen-presenting cells (APCs) (including cDC1, cDC2 and moDCs) was tested in vitro. To this aim, day-7 DCPs were sorted by fluorescence-activated cell sorting (FACS) as CD34⁺ and CD115⁺ cells (FIG. 13F-13G) and allowed to differentiate for additional 7 days in StemSpam SFEMII medium (Stem Cell Technologies; 09605) supplemented with 50 units/ml penicillin (Gibco), 50 g/ml streptomycin (Gibco), 20 ng/ml GM-CSF (Peproteh; 300-03), 100 ng/mL FLT3L (Peprotech; 300-19), 20 ng/mL SCF (Peprotech; 300-07), and 10 ng/mL (1000 IU/mL) IFNa2b (Invivogen; rcyc-hifna2b). Human DCPs gave rise mainly to APCs (cDC1s: CD66b⁻, CD3⁻, CD19⁻, CD14⁻, CD141⁺, and CLEC9A⁺; cDC2s: CD66b⁻, CD3⁻, CD19⁻, CD14⁻, CD141⁻, CLEC9A⁻, and CD1c⁺; monocytes: CD66b⁻, CD3⁻, CD19⁻, and CD14⁺; and immature DCs: CD66b⁻, CD3⁻, CD19⁻, CD14⁻, CD141⁺, and CLEC9A⁻), whereas mock-sorted cells differentiated into a broader range of cell types (including granulocytes: CD66b⁺; T cells: CD66b⁻ and CD3⁺; B cells: CD66b⁻, CD3⁻ and CD19⁺; and other cells: CD66b⁻, CD3⁻, CD19⁻, CD14⁻, CD141⁻, CLEC9A⁻, and CD1c⁻) (FIG. 13H), demonstrating that CD34⁺ and CD115⁺ human DCPs are indeed dendritic cell progenitors.

Statistical analysis: h) Two-way ANOVA with Sidak's multiple comparison test. P values are coded as ****: P<0.0001.

This example thus demonstrates that human DCPs with the ability to differentiate into bona fide antigen-presenting cells comprising cDC1 and cDC2 can be generated from CD34⁺ human hematopoietic stem progenitor cells.

### Example 14: DCP-Derived DCs Outperform moDCs in Antigen Presentation Capacity

To further demonstrate that human DCPs are a source of professional APCs, the antigen-presenting capacity of their progeny (DCP-progeny), obtained as shown in example 13 (FIG. 13F), was assessed and compared to traditional moDCs. To generate human moDCs, blood from healthy human donors was obtained from the Blood Transfusion Center (Lausanne, Switzerland) and peripheral blood mononuclear cells (PBMCs) were isolated by density gradient centrifugation on Lymphoprep (Stem Cell Technologies; 07801). CD14 monocytes were subsequently isolated with magnetic beads (Miltenyi; 130-050-201) following the manufacturer's instructions and cultured in 10% FBS (Gibco; 10270106), 100 U/Ml penicilin, 100 g/mL streptomycin (Gibco; 15140-122), 2 mM glutamine (Gibco; 25030-024), 50 ng/mL GM-CSF (Peprotech; 300-03), 50 ng/mL IL4 (Peprotech; 200-04) in RPMI 1640 (Gibco; 21875-034) at a concentration of 10⁶ cell/mL.

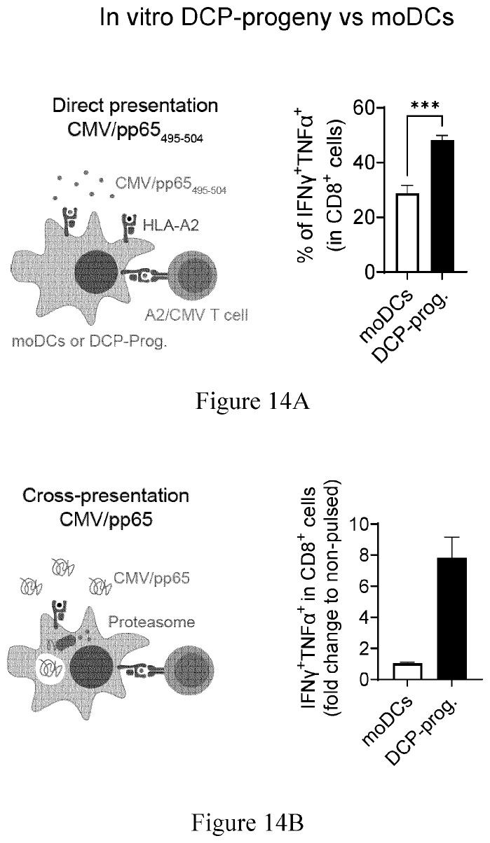

Cytomegalovirus (CMV) protein pp65 (or its HLA-A2-restricted peptide, pp65₄₉₅₋₅₀₄) and HLA-A2-restricted, CMV-specific T cells were used to assess antigen presentation. Two antigen-presentation pathways were examined: (i) presentation of pp65₄₉₅₋₅₀₄ peptide-loaded HLA-A2, which mimics direct presentation and (ii) cross-presentation of the pp65₄₉₅₋₅₀₄ peptide endogenously processed from the native pp65 protein. T cells were co-cultured in the presence of DCP-progeny or moDCs that were previously exposed to pp65₄₉₅₋₅₀₄ (at 1 μg/mL for 1 h at 37° C.) or pp65 protein (ab43041 Abcam, at 10 μg/mL for 2 h at 37° C.), to respectively assay direct presentation and cross-presentation. Pulsed DCP-progeny and moDCs were washed with 10% FBS (Gibco; 10270106), 100 U/Ml penicilin, 100 g/mL streptomycin (15140-122, Gibco) in RPMI 1640 (Gibco; 21875-034) before culturing them with A2/CMV/pp65₄₉₅₋₅₀₄-specific CD8⁺ T cells at 1:1 ratio. Co-cultures were kept overnight at 37° C. and 4 h in the presence of Brefeldin A (1:1000; BD Biosciences, GolgiPlug, 51-2301KZ), before staining for flow cytometry analysis. In each instance, DCP-progeny were superior to moDCs, as shown by the presence of increased IFNγ and TNFα-producing CMV-specific CD8⁺ T cells after co-culture (FIGS. 14A-14B). Of note, moDCs lacked cross-presentation capacity. Thus, these data indicate that human DCPs have the ability to produce progeny with antigen-presentation capacity superior to that of traditional moDCs.

Statistical analysis: a) Unpaired t-test. P value is coded as ***: P<0.001.

This example thus demonstrates that antigen-presenting cells derived from enriched human DCPs have antigen presentation capacity that is superior to that of traditional human monocyte-derived DCs.

### Example 15: Efficient Generation of Transduced Human DCPs

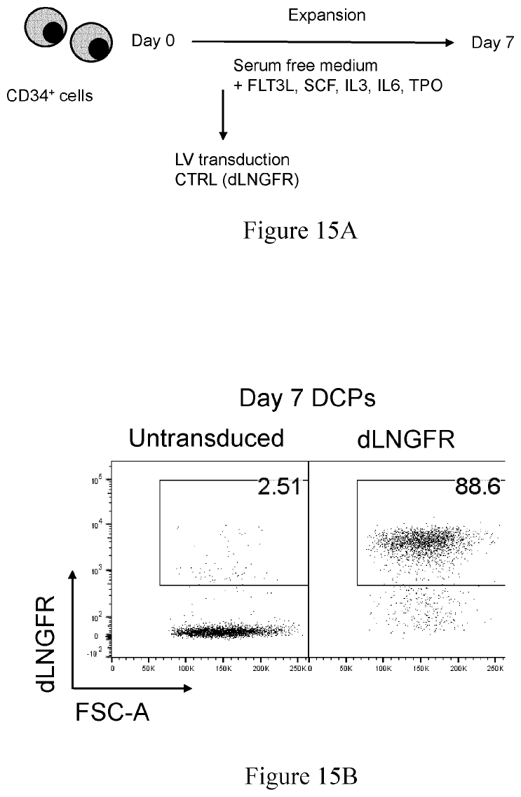

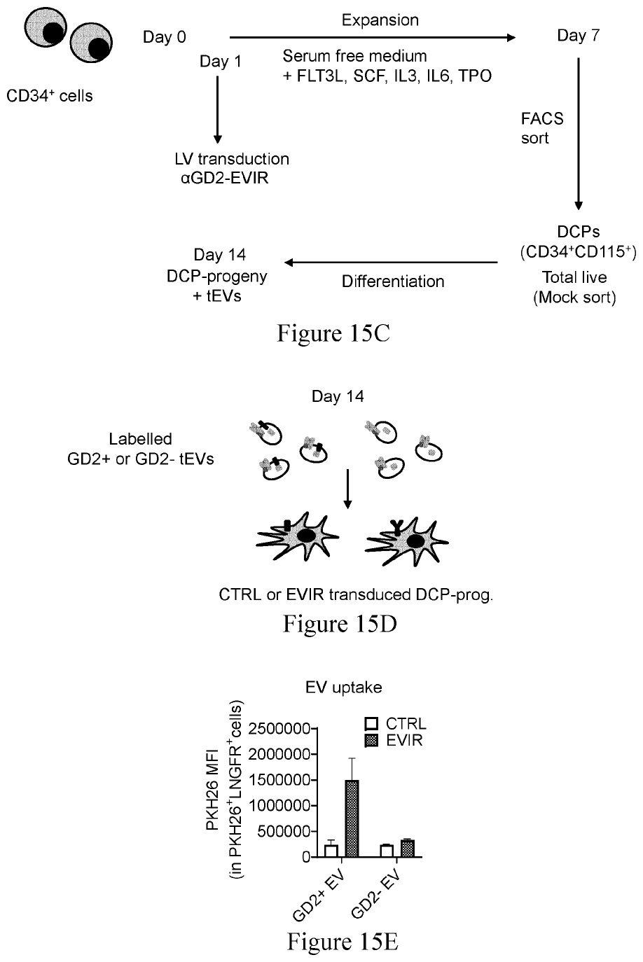

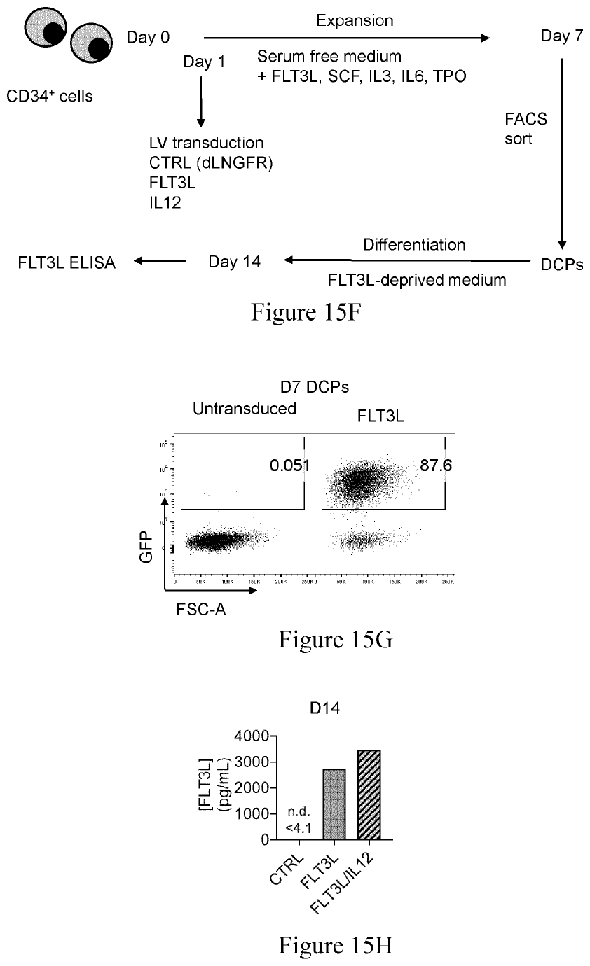

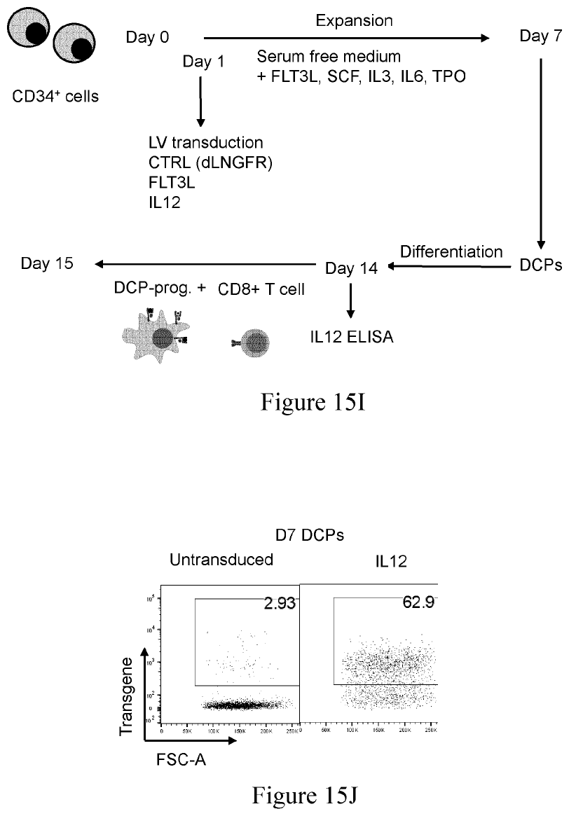

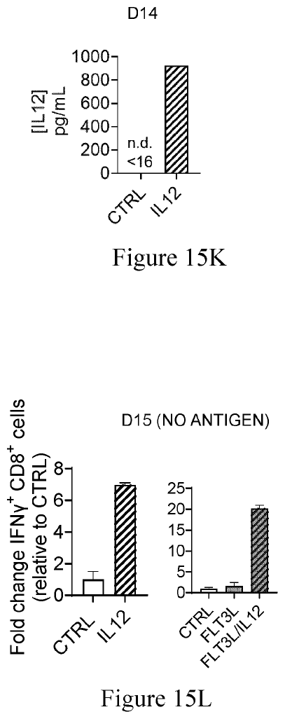

Human DCP transduction was performed at day 1 using concentrated lentiviral vectors (LVs) (FIG. 15A). Before transduction, cells were transferred to retronectin (Takara; T100A) coated wells and dmPGE2 (Stem Cell Technologies; 72372) was added to a final concentration of 10 μM. After 2 h, the cells were transduced with the control dLNGFR-encoding LV at 300 MOI. This protocol generated >80% transgene-expressing DCPs, assessed by flow cytometry (FIG. 15B). DCPs were then transduced with the functional anti-GD2 EVIR. The functionality of GD2-EVIR-DCPs was analyzed by assessing the capacity of DCPs transduced with GD2-EVIR, compared with CTRL dLNGFR, to uptake GD2⁺ or GD2⁻ tumor EVs. Tumor EVs (tEVs) were isolated from human melanoma cell lines expanded in 10% FBS (Gibco; 10270106), 1.1 M arginine (Sigma Aldrich), 0.48 M asparagine (Sigma Aldrich), 11.25 M glutamine (Gibco; 25030-024), 10 mM Hepes (Gibco; 15630-056), 100 U/Ml penicilin, and 100 g/mL sptreptomycin (Gibco; 15140-122) in RPMI 1640 (Gibco; 21875-034). For EV isolation, medium of cells at 20% confluency was replaced with the aforementioned medium modified to contain 5% EV-depleted FBS (obtained by ultracentrifugation of standard FBS at 134,000 g for 16 h at 4° C. followed by filtration through a 0.1 μm vacuum filtration bottle). After 4 days in culture, medium was harvested for EV isolation by sequential ultracentrifugation: medium was centrifuged at 500×g for 5 min, 200×g for 10 min, and 10000×g for 30 min, at 4° C. to remove dead cells and debris. The medium was then ultracentrifuged at 134000×g for 70 min at 4° C. using a Hitachi CP80NX ultracentrifuge. The pellet was washed with 35 mL of PBS and ultracentrifuged again at 134000×g for 70 min at 4° C. to finally resuspend them in PBS. GD2⁺ or GD2⁻ EVs were labelled with the membrane dye PKH26 (1:200, MIDI26-1KT, Sigma, PE) for 10 min at RT and washed them twice with 0.1% BSA in PBS and once with PBS on Vivaspin 500 (300,000 MWCO PES, VS0152 Sartorius) before adding them at 5 μg/mL concentration to CTRL (dLNGFR)-transduced or GD2-EVIR-transduced DCP-progeny (FIG. 15C-15D). The mean fluorescence intensity of PKH26 in transduced DCPs, measured the following day by flow cytometry, indicated enhanced uptake of GD2⁺ tumor EVs by GD2-EVIR-transduced DCP-progeny compared to CTRL-transduced DCP-progeny, indicating that EVIR expression in human DCPs specifically enhances the uptake and internalization of GD2⁻ positive tEVs (FIG. 15E).

DCPs were then transduced with LVs encoding FLT3L and IL-12. DCPs were transduced with FLT3L-GFP, IL12-dLNGFR or dLNGFR coding LVs on day 1, and sorted on day 7 for subsequent culture in FLT3L-deprived StemSpam SFEMII medium (Stem Cell Technologies; 09605) supplemented with 50 units/ml penicillin (Gibco), 50 g/ml streptomycin (Gibco), 20 ng/ml GM-CSF (Peproteh; 300-03), 20 ng/mL SCF (Peprotech; 300-07), and 10 ng/mL (1000 IU/mL) IFNa2b (Invivogen; rcyc-hifna2b) for 7 additional days (FIG. 15F). Day 7 DCPs identified as CD34⁺ and CD115⁺ cells robustly expressed GFP from the FLT3L-GFP LV (FIG. 15G). Also, ELISA analysis (EHFL3LG, Thermo Fisher) of day 14 culture supernatants revealed efficient FLT3L production by FLT3L-transduced cells or a mixture of FLT3L⁻ and IL12-transduced DCPs (2:1 ratio) (FIG. 15H).

The capacity of IL12 to enhance T cell stimulation capacity of human DCPs was assessed (FIG. 15I). dLNGFR analysis by flow cytometry revealed efficient transduction with IL12-dLNGFR LVs on day 7 (FIG. 15J). Moreover, IL12 was detected by ELISA (431701, Biolegend) in the culture supernatants of IL12-transduced DCP-progeny after 7 days of differentiation in StemSpam SFEMII medium (Stem Cell Technologies; 09605) supplemented with 50 units/ml penicillin (Gibco), 50 g/ml streptomycin (Gibco), 20 ng/ml GM-CSF (Peproteh; 300-03), 100 ng/mL FLT3L (Peprotech; 300-19), 20 ng/mL SCF (Peprotech; 300-07), and 10 ng/mL (1000 IU/mL) IFNa2b (Invivogen; rcyc-hifna2b) (FIG. 15K). Notably, IL12-transduced DCP-progeny or a mixture of FLT3L/IL12-transduced (2:1 ratio) DCP-progeny induced antigen-independent IFNγ production by CMV-specific T cells cocultured with DCP-progeny (FIG. 15L).

In summary, the present disclosure demonstrates that human DCPs, like the mouse DCPs of the previous examples, can be armed with effectors (such as EVIR, FLT3L or IL12) to produce a DCP progeny, and these human DCP progeny may recapitulate the anti-tumoral immune functions of mouse DCPs.

List of sequences
GFP
(SEQ ID NO: 1)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAA
(SEQ ID NO: 2)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYK
mIL2-P2A-GFP
(SEQ ID NO: 3)
ATGTACAGCATGCAGCTCGCATCCTGTGTCACATTGACACTTGTGCTCCT
TGTCAACAGCGCACCCACTTCAAGCTCCACTTCAAGCTCTACAGCGGAAG
CACAGCAGCAGCAGCAGCAGCAGCAGCAGCAGCAGCAGCACCTGGAGCAG
CTGTTGATGGACCTACAGGAGCTCCTGAGCAGGATGGAGAATTACAGGAA
CCTGAAACTCCCCAGGATGCTCACCTTCAAATTTTACTTGCCCAAGCAGG
CCACAGAATTGAAAGATCTTCAGTGCCTAGAAGATGAACTTGGACCTCTG
CGGCATGTTCTGGATTTGACTCAAAGCAAAAGCTTTCAATTGGAAGATGC
TGAGAATTTCATCAGCAATATCAGAGTAACTGTTGTAAAACTAAAGGGCT
CTGACAACACATTTGAGTGCCAATTCGATGATGAGTCAGCAACTGTGGTG
GACTTTCTGAGGAGATGGATAGCCTTCTGTCAAAGCATCATCTCAACAAG
CCCTCAAACCGGTTCCGGAGCCACGAACTTCTCTCTGTTAAAGCAAGCAG
GAGACGTGGAAGAAAACCCCGGTCCTATGGTGAGCAAGGGCGAGGAGCTG
TTCACCGGGGTGGTGCCCATCCTGGTCGAGCTGGACGGCGACGTAAACGG
CCACAAGTTCAGCGTGTCCGGCGAGGGCGAGGGCGATGCCACCTACGGCA
AGCTGACCCTGAAGTTCATCTGCACCACCGGCAAGCTGCCCGTGCCCTGG
CCCACCCTCGTGACCACCCTGACCTACGGCGTGCAGTGCTTCAGCCGCTA
CCCCGACCACATGAAGCAGCACGACTTCTTCAAGTCCGCCATGCCCGAAG
GCTACGTCCAGGAGCGCACCATCTTCTTCAAGGACGACGGCAACTACAAG
ACCCGCGCCGAGGTGAAGTTCGAGGGCGACACCCTGGTGAACCGCATCGA
GCTGAAGGGCATCGACTTCAAGGAGGACGGCAACATCCTGGGGCACAAGC
TGGAGTACAACTACAACAGCCACAACGTCTATATCATGGCCGACAAGCAG
AAGAACGGCATCAAGGTGAACTTCAAGATCCGCCACAACATCGAGGACGG
CAGCGTGCAGCTCGCCGACCACTACCAGCAGAACACCCCCATCGGCGACG
GCCCCGTGCTGCTGCCCGACAACCACTACCTGAGCACCCAGTCCGCCCTG
AGCAAAGACCCCAACGAGAAGCGCGATCACATGGTCCTGCTGGAGTTCGT
GACCGCCGCCGGGATCACTCTCGGCATGGACGAGCTGTACAAGTAAGTCG
AC
(SEQ ID NO: 4)
MYSMQLASCVTLTLVLLVNSAPTSSSTSSSTAEAQQQQQQQQQQQQHLEQ
LLMDLQELLSRMENYRNLKLPRMLTFKFYLPKQATELKDLQCLEDELGPL
RHVLDLTQSKSFQLEDAENFISNIRVTVVKLKGSDNTFECQFDDESATVV
DFLRRWIAFCQSIISTSPQTGSGATNFSLLKQAGDVEENPGPMVSKGEEL
FTGVVPILVELDGDVNGHKFSVSGEGEGDATYGKLTLKFICTTGKLPVPW
PTLVTTLTYGVQCFSRYPDHMKQHDFFKSAMPEGYVQERTIFFKDDGNYK
TRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSAL
SKDPNEKRDHMVLLEFVTAAGITLGMDELYK*
mIL12-P2A-GFP
(SEQ ID NO: 5)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGGGTCC
GATCCGGCGGCGGCGGGAGTGGCGGCGGGGGTTCTGGCGGAGGCCTCGCT
AGCGGTGGCTCCATGGTCAGCGTTCCAACAGCCTCACCCTCGGCATCCAG
CAGCTCCTCTCAGTGCCGGTCCAGCATGTGTCAATCACGCTACCTCCTCT
TTTTGGCCACCCTTGCCCTCCTAAACCACCTCAGTTTGGCCAGGGTCATT
CCAGTCTCTGGACCTGCCAGGTGTCTTAGCCAGTCCCGAAACCTGCTGAA
GACCACAGATGACATGGTGAAGACGGCCAGAGAAAAACTGAAACATTATT
CCTGCACTGCTGAAGACATCGATCATGAAGACATCACACGGGACCAAACC
AGCACATTGAAGACCTGTTTACCACTGGAACTACACAAGAACGAGAGTTG
CCTGGCTACTAGAGAGACTTCTTCCACAACAAGAGGGAGCTGCCTGCCCC
CACAGAAGACGTCTTTGATGATGACCCTGTGCCTTGGTAGCATCTATGAG
GACTTGAAGATGTACCAGACAGAGTTCCAGGCCATCAACGCAGCACTTCA
GAATCACAACCATCAGCAGATCATTCTAGACAAGGGCATGCTGGTGGCCA
TCGATGAGCTGATGCAGTCTCTGAATCATAATGGCGAGACTCTGCGCCAG
AAACCTCCTGTGGGAGAAGCAGACCCTTACAGAGTGAAAATGAAGCTCTG
CATCCTGCTTCACGCCTTCAGCACCCGCGTCGTGACCATCAACAGGGTGA
TGGGCTATCTGAGCTCCGCCACCGGTTCCGGAGCCACGAACTTCTCTCTG
TTAAAGCAAGCAGGAGACGTGGAAGAAAACCCCGGTCCTATGGTGAGCAA
GGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCT
GCCCGTGCCCTGGCCCACCCTCGTGACCACCCTGACCTACGGCGTGCAGT
GCTTCAGCCGCTACCCCGACCACATGAAGCAGCACGACTTCTTCAAGTCC
GCCATGCCCGAAGGCTACGTCCAGGAGCGCACCATCTTCTTCAAGGACGA
CGGCAACTACAAGACCCGCGCCGAGGTGAAGTTCGAGGGCGACACCCTGG
TGAACCGCATCGAGCTGAAGGGCATCGACTTCAAGGAGGACGGCAACATC
CTGGGGCACAAGCTGGAGTACAACTACAACAGCCACAACGTCTATATCAT
GGCCGACAAGCAGAAGAACGGCATCAAGGTGAACTTCAAGATCCGCCACA
ACATCGAGGACGGCAGCGTGCAGCTCGCCGACCACTACCAGCAGAACACC
CCCATCGGCGACGGCCCCGTGCTGCTGCCCGACAACCACTACCTGAGCAC
CCAGTCCGCCCTGAGCAAAGACCCCAACGAGAAGCGCGATCACATGGTCC
TGCTGGAGTTCGTGACCGCCGCCGGGATCACTCTCGGCATGGACGAGCTG
TACAAGTAA
(SEQ ID NO: 6)
MCPQKLTISWFAIVLLVSPLMAMWELEKDVYVVEVDWTPDAPGETVNLTC
DTPEEDDITWTSDQRHGVIGSGKTLTITVKEFLDAGQYTCHKGGETLSHS
HLLLHKKENGIWSTEILKNFKNKTFLKCEAPNYSGRFTCSWLVQRNMDLK
FNIKSSSSSPDSRAVTCGMASLSAEKVTLDQRDYEKYSVSCQEDVTCPTA
EETLPIELALEARQQNKYENYSTSFFIRDIIKPDPPKNLQMKPLKNSQVE
VSWEYPDSWSTPHSYFSLKFFVRIQRKKEKMKETEEGCNQKGAFLVEKTS
TEVQCKGGNVCVQAQDRYYNSSCSKWACVPCRVRSGGGGSGGGGSGGGLA
SGGSMVSVPTASPSASSSSSQCRSSMCQSRYLLFLATLALLNHLSLARVI
PVSGPARCLSQSRNLLKTTDDMVKTAREKLKHYSCTAEDIDHEDITRDQT
STLKTCLPLELHKNESCLATRETSSTTRGSCLPPQKTSLMMTLCLGSIYE
DLKMYQTEFQAINAALQNHNHQQIILDKGMLVAIDELMQSLNHNGETLRQ
KPPVGEADPYRVKMKLCILLHAFSTRVVTINRVMGYLSSATGSGATNFSL
LKQAGDVEENPGPMVSKGEELFTGVVPILVELDGDVNGHKFSVSGEGEGD
ATYGKLTLKFICTTGKLPVPWPTLVTTLTYGVQCFSRYPDHMKQHDFFKS
AMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNT
PIGDGPVLLPDNHYLSTQSALSKDPNEKRDHMVLLEFVTAAGITLGMDEL
YK*
dOVA
(SEQ ID NO: 7)
ATGGACAGCACCAGGACACAAATAAATAAGGTTGTTCGCTTTGATAAACT
TCCAGGATTCGGAGACAGTATTGAAGCTCAGTGTGGCACATCTGTAAACG
TTCACTCTTCACTTAGAGACATCCTCAACCAAATCACCAAACCAAATGAT
GTTTATTCGTTCAGCCTTGCCAGTAGACTTTATGCTGAAGAGAGATACCC
AATCCTGCCAGAATACTTGCAGTGTGTGAAGGAACTGTATAGAGGAGGCT
TGGAACCTATCAACTTTCAAACAGCTGCAGATCAAGCCAGAGAGCTCATC
AATTCCTGGGTAGAAAGTCAGACAAATGGAATTATCAGAAATGTCCTTCA
GCCAAGCTCCGTGGATTCTCAAACTGCAATGGTTCTGGTTAATGCCATTG
TCTTCAAAGGACTGTGGGAGAAAGCATTTAAGGATGAAGACACACAAGCA
ATGCCTTTCAGAGTGACTGAGCAAGAAAGCAAACCTGTGCAGATGATGTA
CCAGATTGGTTTATTTAGAGTGGCATCAATGGCTTCTGAGAAAATGAAGA
TCCTGGAGCTTCCATTTGCCAGTGGGACAATGAGCATGTTGGTGCTGTTG
CCTGATGAAGTCTCAGGCCTTGAGCAGCTTGAGAGTATAATCAACTTTGA
AAAACTGACTGAATGGACCAGTTCTAATGTTATGGAAGAGAGGAAGATCA
AAGTGTACTTACCTCGCATGAAGATGGAGGAAAAATACAACCTCACATCT
GTCTTAATGGCTATGGGCATTACTGACGTGTTTAGCTCTTCAGCCAATCT
GTCTGGCATCTCCTCAGCAGAGAGCCTGAAGATATCTCAAGCTGTCCATG
CAGCACATGCAGAAATCAATGAAGCAGGCAGAGAGGTGGTAGGGTCAGCA
GAGGCTGGAGTGGATGCTGCAAGCGTCTCTGAAGAATTTAGGGCTGACCA
TCCATTCCTCTTCTGTATCAAGCACATCGCAACCAACGCCGTTCTCTTCT
TTGGCAGATGTGTTTCCCCTGGCGGCGGCTGA
(SEQ ID NO: 8)
MDSTRTQINKVVRFDKLPGFGDSIEAQCGTSVNVHSSLRDILNQITKPND
VYSFSLASRLYAEERYPILPEYLQCVKELYRGGLEPINFQTAADQARELI
NSWVESQINGIIRNVLQPSSVDSQTAMVLVNAIVFKGLWEKAFKDEDTQA
MPFRVTEQESKPVQMMYQIGLFRVASMASEKMKILELPFASGTMSMLVLL
PDEVSGLEQLESIINFEKLTEWTSSNVMEERKIKVYLPRMKMEEKYNLTS
VLMAMGITDVFSSSANLSGISSAESLKISQAVHAAHAEINEAGREVVGSA
EAGVDAASVSEEFRADHPFLFCIKHIATNAVLFFGRCVSPGGG*
mIL2-P2A-dOVA
(SEQ ID NO: 9)
ATGTACAGCATGCAGCTCGCATCCTGTGTCACATTGACACTTGTGCTCCT
TGTCAACAGCGCACCCACTTCAAGCTCCACTTCAAGCTCTACAGCGGAAG
CACAGCAGCAGCAGCAGCAGCAGCAGCAGCAGCAGCAGCACCTGGAGCAG
CTGTTGATGGACCTACAGGAGCTCCTGAGCAGGATGGAGAATTACAGGAA
CCTGAAACTCCCCAGGATGCTCACCTTCAAATTTTACTTGCCCAAGCAGG
CCACAGAATTGAAAGATCTTCAGTGCCTAGAAGATGAACTTGGACCTCTG
CGGCATGTTCTGGATTTGACTCAAAGCAAAAGCTTTCAATTGGAAGATGC
TGAGAATTTCATCAGCAATATCAGAGTAACTGTTGTAAAACTAAAGGGCT
CTGACAACACATTTGAGTGCCAATTCGATGATGAGTCAGCAACTGTGGTG
GACTTTCTGAGGAGATGGATAGCCTTCTGTCAAAGCATCATCTCAACAAG
CCCTCAAACCGGTTCCGGAGCCACGAACTTCTCTCTGTTAAAGCAAGCAG
GAGACGTGGAAGAAAACCCCGGTCCTACGCGTATGGACAGCACCAGGACA
CAAATAAATAAGGTTGTTCGCTTTGATAAACTTCCAGGATTCGGAGACAG
TATTGAAGCTCAGTGTGGCACATCTGTAAACGTTCACTCTTCACTTAGAG
ACATCCTCAACCAAATCACCAAACCAAATGATGTTTATTCGTTCAGCCTT
GCCAGTAGACTTTATGCTGAAGAGAGATACCCAATCCTGCCAGAATACTT
GCAGTGTGTGAAGGAACTGTATAGAGGAGGCTTGGAACCTATCAACTTTC
AAACAGCTGCAGATCAAGCCAGAGAGCTCATCAATTCCTGGGTAGAAAGT
CAGACAAATGGAATTATCAGAAATGTCCTTCAGCCAAGCTCCGTGGATTC
TCAAACTGCAATGGTTCTGGTTAATGCCATTGTCTTCAAAGGACTGTGGG
AGAAAGCATTTAAGGATGAAGACACACAAGCAATGCCTTTCAGAGTGACT
GAGCAAGAAAGCAAACCTGTGCAGATGATGTACCAGATTGGTTTATTTAG
AGTGGCATCAATGGCTTCTGAGAAAATGAAGATCCTGGAGCTTCCATTTG
CCAGTGGGACAATGAGCATGTTGGTGCTGTTGCCTGATGAAGTCTCAGGC
CTTGAGCAGCTTGAGAGTATAATCAACTTTGAAAAACTGACTGAATGGAC
CAGTTCTAATGTTATGGAAGAGAGGAAGATCAAAGTGTACTTACCTCGCA
TGAAGATGGAGGAAAAATACAACCTCACATCTGTCTTAATGGCTATGGGC
ATTACTGACGTGTTTAGCTCTTCAGCCAATCTGTCTGGCATCTCCTCAGC
AGAGAGCCTGAAGATATCTCAAGCTGTCCATGCAGCACATGCAGAAATCA
ATGAAGCAGGCAGAGAGGTGGTAGGGTCAGCAGAGGCTGGAGTGGATGCT
GCAAGCGTCTCTGAAGAATTTAGGGCTGACCATCCATTCCTCTTCTGTAT
CAAGCACATCGCAACCAACGCCGTTCTCTTCTTTGGCAGATGTGTTTCCC
CTGGCGGCGGCTGA
(SEQ ID NO: 10)
MYSMQLASCVTLTLVLLVNSAPTSSSTSSSTAEAQQQQQQQQQQQQHLEQ
LLMDLQELLSRMENYRNLKLPRMLTFKFYLPKQATELKDLQCLEDELGPL
RHVLDLTQSKSFQLEDAENFISNIRVTVVKLKGSDNTFECQFDDESATVV
DFLRRWIAFCQSIISTSPQTGSGATNFSLLKQAGDVEENPGPTRMDSTRT
QINKVVRFDKLPGFGDSIEAQCGTSVNVHSSLRDILNQITKPNDVYSFSL
ASRLYAEERYPILPEYLQCVKELYRGGLEPINFQTAADQARELINSWVES
QTNGIIRNVLQPSSVDSQTAMVLVNAIVFKGLWEKAFKDEDTQAMPFRVT
EQESKPVQMMYQIGLFRVASMASEKMKILELPFASGTMSMLVLLPDEVSG
LEQLESIINFEKLTEWTSSNVMEERKIKVYLPRMKMEEKYNLTSVLMAMG
ITDVFSSSANLSGISSAESLKISQAVHAAHAEINEAGREVVGSAEAGVDA
ASVSEEFRADHPFLFCIKHIATNAVLFFGRCVSPGGG*
mIL12-P2A-dOVA
(SEQ ID NO: 11)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGGGTCC
GATCCGGCGGCGGCGGGAGTGGCGGCGGGGGTTCTGGCGGAGGCCTCGCT
AGCGGTGGCTCCATGGTCAGCGTTCCAACAGCCTCACCCTCGGCATCCAG
CAGCTCCTCTCAGTGCCGGTCCAGCATGTGTCAATCACGCTACCTCCTCT
TTTTGGCCACCCTTGCCCTCCTAAACCACCTCAGTTTGGCCAGGGTCATT
CCAGTCTCTGGACCTGCCAGGTGTCTTAGCCAGTCCCGAAACCTGCTGAA
GACCACAGATGACATGGTGAAGACGGCCAGAGAAAAACTGAAACATTATT
CCTGCACTGCTGAAGACATCGATCATGAAGACATCACACGGGACCAAACC
AGCACATTGAAGACCTGTTTACCACTGGAACTACACAAGAACGAGAGTTG
CCTGGCTACTAGAGAGACTTCTTCCACAACAAGAGGGAGCTGCCTGCCCC
CACAGAAGACGTCTTTGATGATGACCCTGTGCCTTGGTAGCATCTATGAG
GACTTGAAGATGTACCAGACAGAGTTCCAGGCCATCAACGCAGCACTTCA
GAATCACAACCATCAGCAGATCATTCTAGACAAGGGCATGCTGGTGGCCA
TCGATGAGCTGATGCAGTCTCTGAATCATAATGGCGAGACTCTGCGCCAG
AAACCTCCTGTGGGAGAAGCAGACCCTTACAGAGTGAAAATGAAGCTCTG
CATCCTGCTTCACGCCTTCAGCACCCGCGTCGTGACCATCAACAGGGTGA
TGGGCTATCTGAGCTCCGCCACCGGTTCCGGAGCCACGAACTTCTCTCTG
TTAAAGCAAGCAGGAGACGTGGAAGAAAACCCCGGTCCTACGCGTATGGA
CAGCACCAGGACACAAATAAATAAGGTTGTTCGCTTTGATAAACTTCCAG
GATTCGGAGACAGTATTGAAGCTCAGTGTGGCACATCTGTAAACGTTCAC
TCTTCACTTAGAGACATCCTCAACCAAATCACCAAACCAAATGATGTTTA
TTCGTTCAGCCTTGCCAGTAGACTTTATGCTGAAGAGAGATACCCAATCC
TGCCAGAATACTTGCAGTGTGTGAAGGAACTGTATAGAGGAGGCTTGGAA
CCTATCAACTTTCAAACAGCTGCAGATCAAGCCAGAGAGCTCATCAATTC
CTGGGTAGAAAGTCAGACAAATGGAATTATCAGAAATGTCCTTCAGCCAA
GCTCCGTGGATTCTCAAACTGCAATGGTTCTGGTTAATGCCATTGTCTTC
AAAGGACTGTGGGAGAAAGCATTTAAGGATGAAGACACACAAGCAATGCC
TTTCAGAGTGACTGAGCAAGAAAGCAAACCTGTGCAGATGATGTACCAGA
TTGGTTTATTTAGAGTGGCATCAATGGCTTCTGAGAAAATGAAGATCCTG
GAGCTTCCATTTGCCAGTGGGACAATGAGCATGTTGGTGCTGTTGCCTGA
TGAAGTCTCAGGCCTTGAGCAGCTTGAGAGTATAATCAACTTTGAAAAAC
TGACTGAATGGACCAGTTCTAATGTTATGGAAGAGAGGAAGATCAAAGTG
TACTTACCTCGCATGAAGATGGAGGAAAAATACAACCTCACATCTGTCTT
AATGGCTATGGGCATTACTGACGTGTTTAGCTCTTCAGCCAATCTGTCTG
GCATCTCCTCAGCAGAGAGCCTGAAGATATCTCAAGCTGTCCATGCAGCA
CATGCAGAAATCAATGAAGCAGGCAGAGAGGTGGTAGGGTCAGCAGAGGC
TGGAGTGGATGCTGCAAGCGTCTCTGAAGAATTTAGGGCTGACCATCCAT
TCCTCTTCTGTATCAAGCACATCGCAACCAACGCCGTTCTCTTCTTTGGC
AGATGTGTTTCCCCTGGCGGCGGCTGA
(SEQ ID NO: 12)
MCPQKLTISWFAIVLLVSPLMAMWELEKDVYVVEVDWTPDAPGETVNLTC
DTPEEDDITWTSDQRHGVIGSGKTLTITVKEFLDAGQYTCHKGGETLSHS
HLLLHKKENGIWSTEILKNFKNKTFLKCEAPNYSGRFTCSWLVQRNMDLK
FNIKSSSSSPDSRAVTCGMASLSAEKVTLDQRDYEKYSVSCQEDVTCPTA
EETLPIELALEARQQNKYENYSTSFFIRDIIKPDPPKNLQMKPLKNSQVE
VSWEYPDSWSTPHSYFSLKFFVRIQRKKEKMKETEEGCNQKGAFLVEKTS
TEVQCKGGNVCVQAQDRYYNSSCSKWACVPCRVRSGGGGSGGGGSGGGLA
SGGSMVSVPTASPSASSSSSQCRSSMCQSRYLLFLATLALLNHLSLARVI
PVSGPARCLSQSRNLLKTTDDMVKTAREKLKHYSCTAEDIDHEDITRDQT
STLKTCLPLELHKNESCLATRETSSTTRGSCLPPQKTSLMMTLCLGSIYE
DLKMYQTEFQAINAALQNHNHQQIILDKGMLVAIDELMQSLNHNGETLRQ
KPPVGEADPYRVKMKLCILLHAFSTRVVTINRVMGYLSSATGSGATNFSL
LKQAGDVEENPGPTRMDSTRTQINKVVRFDKLPGFGDSIEAQCGTSVNVH
SSLRDILNQITKPNDVYSFSLASRLYAEERYPILPEYLQCVKELYRGGLE
PINFQTAADQARELINSWVESQINGIIRNVLQPSSVDSQTAMVLVNAIVF
KGLWEKAFKDEDTQAMPFRVTEQESKPVQMMYQIGLFRVASMASEKMKIL
ELPFASGTMSMLVLLPDEVSGLEQLESIINFEKLTEWTSSNVMEERKIKV
YLPRMKMEEKYNLTSVLMAMGITDVFSSSANLSGISSAESLKISQAVHAA
HAEINEAGREVVGSAEAGVDAASVSEEFRADHPFLFCIKHIATNAVLFFG
RCVSPGGG*
dLNGFR
(SEQ ID NO: 13)
ATGGACGGGCCGCGCCTGCTGCTGTTGCTGCTTCTGGGGGTGTCCCTTGG
AGGTGCCAAGGAGGCATGCCCCACAGGCCTGTACACACACAGCGGTGAGT
GCTGCAAAGCCTGCAACCTGGGCGAGGGTGTGGCCCAGCCTTGTGGAGCC
AACCAGACCGTGTGTGAGCCCTGCCTGGACAGCGTGACGTTCTCCGACGT
GGTGAGCGCGACCGAGCCGTGCAAGCCGTGCACCGAGTGCGTGGGGCTCC
AGAGCATGTCGGCGCCGTGCGTGGAGGCCGACGACGCCGTGTGCCGCTGC
GCCTACGGCTACTACCAGGATGAGACGACTGGGCGCTGCGAGGCGTGCCG
CGTGTGCGAGGCGGGCTCGGGCCTCGTGTTCTCCTGCCAGGACAAGCAGA
ACACCGTGTGCGAGGAGTGCCCCGACGGCACGTATTCCGACGAGGCCAAC
CACGTGGACCCGTGCCTGCCCTGCACCGTGTGCGAGGACACCGAGCGCCA
GCTCCGCGAGTGCACACGCTGGGCCGACGCCGAGTGCGAGGAGATCCCTG
GCCGTTGGATTACACGGTCCACACCCCCAGAGGGCTCGGACAGCACAGCC
CCCAGCACCCAGGAGCCTGAGGCACCTCCAGAACAAGACCTCATAGCCAG
CACGGTGGCAGGTGTGGTGACCACAGTGATGGGCAGCTCCCAGCCCGTGG
TGACCCGAGGCACCACCGACAACCTCATCCCTGTCTATTGCTCCATCCTG
GCTGCTGTGGTTGTGGGCCTTGTGGCCTACATAGCCTTCAAGAGGTGGAA
CAGGGGGATCCTCTAG
(SEQ ID NO: 14)
MDGPRLLLLLLLGVSLGGAKEACPTGLYTHSGECCKACNLGEGVAQPCGA
NQTVCEPCLDSVTFSDVVSATEPCKPCTECVGLQSMSAPCVEADDAVCRC
AYGYYQDETTGRCEACRVCEAGSGLVFSCQDKQNTVCEECPDGTYSDEAN
HVDPCLPCTVCEDTERQLRECTRWADAECEEIPGRWITRSTPPEGSDSTA
PSTQEPEAPPEQDLIASTVAGVVTTVMGSSQPVVTRGTTDNLIPVYCSIL
AAVVVGLVAYIAFKRWNRGIL*
mIL12-P2A-dLNGFR
(SEQ ID NO: 15)
ATGTGCCCTCAGAAGCTCACTATCTCCTGGTTTGCTATTGTCCTCCTGGT
CAGCCCCCTCATGGCTATGTGGGAACTGGAAAAAGACGTGTACGTGGTCG
AGGTGGACTGGACACCCGATGCCCCTGGCGAGACAGTGAACCTGACATGT
GACACCCCCGAGGAGGACGATATCACCTGGACATCCGATCAGAGGCACGG
AGTGATCGGATCTGGCAAGACCCTGACAATCACCGTGAAGGAGTTCCTGG
ACGCTGGCCAGTACACATGCCACAAGGGCGGAGAGACACTGTCTCACAGC
CACCTGCTGCTGCACAAGAAGGAGAACGGAATCTGGTCCACAGAGATCCT
GAAGAACTTCAAGAACAAGACCTTTCTGAAGTGTGAGGCCCCCAACTACT
CTGGCCGGTTCACCTGCAGCTGGCTGGTGCAGCGCAACATGGACCTGAAG
TTTAACATCAAGAGCTCCTCTAGCTCCCCTGATAGCAGGGCTGTGACATG
TGGCATGGCCTCCCTGTCTGCTGAGAAGGTGACCCTGGACCAGAGAGATT
ACGAGAAGTACAGCGTGTCCTGTCAGGAGGACGTGACATGCCCAACCGCC
GAGGAGACACTGCCAATCGAGCTGGCCCTGGAGGCTCGGCAGCAGAACAA
GTACGAGAACTACTCTACCAGCTTCTTTATCCGCGACATCATCAAGCCTG
ATCCCCCTAAGAACCTGCAGATGAAGCCACTGAAGAACTCCCAGGTGGAG
GTGTCTTGGGAGTACCCCGACTCCTGGTCTACACCTCACTCTTACTTCAG
CCTGAAGTTCTTTGTGCGGATCCAGCGCAAGAAGGAGAAGATGAAGGAGA
CAGAGGAGGGATGTAACCAGAAGGGCGCCTTTCTGGTGGAGAAGACAAGC
ACCGAGGTGCAGTGTAAGGGCGGAAACGTGTGCGTGCAGGCCCAGGATCG
GTACTACAACTCTAGCTGCTCCAAGTGGGCTTGCGTGCCTTGTAGGGTGA
GAAGCGGAGGAGGAGGATCCGGAGGAGGAGGATCTGGAGGAGGCCTGGCT
TCCGGAGGATCTATGGTGAGCGTGCCAACAGCTAGCCCTTCCGCTTCCTC
TAGCTCCTCTCAGTGTCGGAGCTCCATGTGCCAGTCCCGCTACCTGCTGT
TCCTGGCCACCCTGGCTCTGCTGAACCACCTGAGCCTGGCTCGCGTGATC
CCAGTGTCCGGACCAGCTAGGTGTCTGTCTCAGAGCCGCAACCTGCTGAA
GACCACAGACGATATGGTGAAGACAGCCCGGGAGAAGCTGAAGCACTACA
GCTGCACCGCTGAGGACATCGATCACGAGGACATCACACGCGATCAGACA
TCTACCCTGAAGACCTGTCTGCCTCTGGAGCTGCACAAGAACGAGAGCTG
CCTGGCCACAAGGGAGACAAGCAGCACCACAAGAGGATCTTGTCTGCCAC
CCCAGAAGACAAGCCTGATGATGACCCTGTGCCTGGGCTCCATCTACGAG
GACCTGAAGATGTACCAGACCGAGTTTCAGGCCATCAACGCCGCTCTGCA
GAACCACAACCACCAGCAGATCATCCTGGACAAGGGCATGCTGGTGGCTA
TCGATGAGCTGATGCAGTCCCTGAACCACAACGGAGAGACACTGAGGCAG
AAGCCTCCAGTGGGCGAGGCCGATCCATACAGAGTGAAGATGAAGCTGTG
CATCCTGCTGCACGCTTTCTCTACAAGGGTGGTGACCATCAACAGAGTGA
TGGGATACCTGTCCTCTGCCACAGGATCCGGCGCTACCAACTTTTCTCTG
CTGAAGCAGGCTGGCGACGTGGAGGAGAACCCTGGACCAATGGATGGACC
TAGGCTCCTCCTGCTGCTGCTGCTGGGCGTGTCCCTGGGAGGAGCTAAGG
AGGCTTGCCCAACAGGACTGTACACCCACTCTGGCGAGTGCTGTAAGGCC
TGTAACCTGGGAGAGGGAGTGGCTCAGCCATGCGGAGCTAACCAGACAGT
GTGCGAGCCTTGTCTGGACTCCGTGACCTTCTCTGACGTGGTGAGCGCCA
CAGAGCCATGCAAGCCCTGTACCGAGTGCGTGGGCCTGCAGTCCATGTCT
GCCCCATGCGTGGAGGCTGACGATGCCGTGTGCAGATGTGCTTACGGATA
CTACCAGGACGAGACAACAGGCAGGTGTGAGGCTTGCAGGGTGTGCGAGG
CTGGAAGCGGCCTGGTGTTTTCCTGCCAGGATAAGCAGAACACAGTGTGC
GAGGAGTGTCCAGACGGAACCTACAGCGATGAGGCCAACCACGTGGACCC
TTGCCTGCCATGTACAGTGTGCGAGGATACCGAGAGGCAGCTGAGAGAGT
GTACCAGATGGGCTGACGCTGAGTGCGAGGAGATCCCAGGACGGTGGATC
ACACGCAGCACCCCACCTGAGGGAAGCGACTCCACAGCTCCTTCCACCCA
GGAGCCAGAGGCTCCACCAGAGCAGGATCTGATCGCCTCTACAGTGGCTG
GAGTGGTGACCACAGTGATGGGCAGCTCCCAGCCAGTGGTGACCAGAGGC
ACCACAGACAACCTGATCCCCGTGTACTGCAGCATCCTGGCCGCTGTGGT
GGTGGGGCTGGTGGCATACATTGCTTTCAAGAGATGGAATAGGGGAATCC
TGTGA
(SEQ ID NO: 16)
MCPQKLTISWFAIVLLVSPLMAMWELEKDVYVVEVDWTPDAPGETVNLTC
DTPEEDDITWTSDQRHGVIGSGKTLTITVKEFLDAGQYTCHKGGETLSHS
HLLLHKKENGIWSTEILKNFKNKTFLKCEAPNYSGRFTCSWLVQRNMDLK
FNIKSSSSSPDSRAVTCGMASLSAEKVTLDQRDYEKYSVSCQEDVTCPTA
EETLPIELALEARQQNKYENYSTSFFIRDIIKPDPPKNLQMKPLKNSQVE
VSWEYPDSWSTPHSYFSLKFFVRIQRKKEKMKETEEGCNQKGAFLVEKTS
TEVQCKGGNVCVQAQDRYYNSSCSKWACVPCRVRSGGGGSGGGGSGGGLA
SGGSMVSVPTASPSASSSSSQCRSSMCQSRYLLFLATLALLNHLSLARVI
PVSGPARCLSQSRNLLKTTDDMVKTAREKLKHYSCTAEDIDHEDITRDQT
STLKTCLPLELHKNESCLATRETSSTTRGSCLPPQKTSLMMTLCLGSIYE
DLKMYQTEFQAINAALQNHNHQQIILDKGMLVAIDELMQSLNHNGETLRQ
KPPVGEADPYRVKMKLCILLHAFSTRVVTINRVMGYLSSATGSGATNFSL
LKQAGDVEENPGPMDGPRLLLLLLLGVSLGGAKEACPTGLYTHSGECCKA
CNLGEGVAQPCGANQTVCEPCLDSVTFSDVVSATEPCKPCTECVGLQSMS
APCVEADDAVCRCAYGYYQDETTGRCEACRVCEAGSGLVFSCQDKQNTVC
EECPDGTYSDEANHVDPCLPCTVCEDTERQLRECTRWADAECEEIPGRWI
TRSTPPEGSDSTAPSTQEPEAPPEQDLIASTVAGVVTTVMGSSQPVVTRG
TTDNLIPVYCSILAAVVVGLVAYIAFKRWNRGIL*
mIL12-P2A-EVIR(anti-GD2)
(SEQ ID NO: 17)
ATGTGCCCTCAGAAACTGACTATCTCCTGGTTTGCTATTGTCCTCCTCGT
GTCCCCTCTCATGGCTATGTGGGAACTGGAAAAAGACGTGTACGTGGTCG
AGGTGGACTGGACCCCAGATGCTCCAGGAGAGACAGTGAACCTGACATGT
GACACCCCTGAGGAGGACGATATCACCTGGACATCTGATCAGAGACACGG
CGTGATCGGAAGCGGCAAGACCCTGACAATCACCGTGAAGGAGTTCCTGG
ACGCCGGACAGTACACCTGCCACAAGGGCGGAGAGACACTGTCTCACAGC
CACCTGCTGCTGCACAAGAAGGAGAACGGCATCTGGAGCACCGAGATCCT
GAAGAACTTCAAGAACAAGACATTTCTGAAGTGTGAGGCCCCCAACTACA
GCGGACGGTTCACCTGCTCCTGGCTGGTGCAGCGCAACATGGACCTGAAG
TTTAACATCAAGAGCTCCTCTAGCTCCCCTGATTCCAGGGCTGTGACCTG
TGGCATGGCCTCCCTGTCTGCTGAGAAGGTGACACTGGACCAGAGAGATT
ACGAGAAGTACAGCGTGTCCTGTCAGGAGGACGTGACATGCCCTACCGCC
GAGGAGACACTGCCAATCGAGCTGGCCCTGGAGGCTCGGCAGCAGAACAA
GTACGAGAACTACTCTACAAGCTTCTTTATCCGCGACATCATCAAGCCCG
ATCCCCCTAAGAACCTGCAGATGAAGCCTCTGAAGAACTCTCAGGTGGAG
GTGAGCTGGGAGTACCCAGACTCCTGGTCTACCCCCCACTCTTACTTCAG
CCTGAAGTTCTTTGTGCGGATCCAGCGCAAGAAGGAGAAGATGAAGGAGA
CAGAGGAGGGCTGTAACCAGAAGGGAGCCTTTCTGGTGGAGAAGACAAGC
ACCGAGGTGCAGTGTAAGGGCGGAAACGTGTGCGTGCAGGCCCAGGATAG
GTACTACAACTCTAGCTGCTCCAAGTGGGCTTGCGTGCCATGTAGGGTGA
GATCCGGAGGAGGAGGATCTGGAGGAGGAGGAAGCGGAGGAGGCCTGGCT
TCCGGAGGCTCTATGGTGAGCGTGCCAACCGCTAGCCCATCCGCTTCCTC
TAGCTCCTCTCAGTGTCGGAGCTCCATGTGCCAGAGCCGCTACCTGCTGT
TCCTGGCCACACTGGCTCTGCTGAACCACCTGTCCCTGGCTAGAGTGATC
CCCGTGAGCGGACCAGCTAGGTGTCTGTCTCAGAGCCGCAACCTGCTGAA
GACCACAGACGATATGGTGAAGACCGCCCGGGAGAAGCTGAAGCACTACT
CCTGCACAGCTGAGGACATCGATCACGAGGACATCACCCGCGATCAGACA
TCCACCCTGAAGACATGTCTGCCCCTGGAGCTGCACAAGAACGAGTCTTG
CCTGGCCACAAGGGAGACAAGCAGCACCACAAGAGGCAGCTGTCTGCCAC
CCCAGAAGACATCCCTGATGATGACCCTGTGCCTGGGAAGCATCTACGAG
GACCTGAAGATGTACCAGACCGAGTTCCAGGCCATCAACGCCGCTCTGCA
GAACCACAACCACCAGCAGATCATCCTGGACAAGGGCATGCTGGTGGCTA
TCGATGAGCTGATGCAGAGCCTGAACCACAACGGCGAGACACTGAGGCAG
AAGCCTCCAGTGGGAGAGGCCGATCCTTACAGAGTGAAGATGAAGCTGTG
CATCCTGCTGCACGCTTTTTCCACAAGGGTGGTGACCATCAACAGAGTGA
TGGGCTACCTGTCCTCTGCCACAGGATCTGGCGCTACCAACTTCAGCCTG
CTGAAGCAGGCTGGCGACGTGGAGGAGAACCCAGGACCTATGGATTTTCA
GGTGCAGATCTTCTCCTTTCTGCTGATCTCCGCTTCTGTGATCATGTCCA
GGGGAGAGGTGCAGCTGCTGCAGTCTGGCCCTGAGCTGGAGAAGCCAGGA
GCCTCCGTGATGATCTCTTGCAAGGCTAGCGGCAGCTCCTTCACCGGATA
CAACATGAACTGGGTGAGGCAGAACATCGGCAAGAGCCTGGAGTGGATCG
GAGCCATCGACCCATACTACGGAGGCACCTCCTACAACCAGAAGTTTAAG
GGCAGAGCCACACTGACCGTGGACAAGTCTAGCTCCACCGCTTACATGCA
CCTGAAGTCCCTGACATCTGAGGATAGCGCCGTGTACTACTGCGTGAGCG
GAATGGAGTACTGGGGACAGGGCACATCCGTGACCGTGTCTAGCGGCGGA
GGAGGCTCTGGCGGCGGCGGCTCCGGCGGAGGAGGAAGCGACGTGGTCAT
GACACAGACCCCACTGTCCCTGCCCGTGAGCCTGGGCGATCAGGCCAGCA
TCTCCTGCAGGTCCTCTCAGTCCCTGGTGCACAGAAACGGCAACACCTAC
CTGCACTGGTACCTGCAGAAGCCAGGACAGTCTCCCAAGCTGCTGATCCA
CAAGGTGAGCAACCGGTTCTCCGGCGTGCCTGACCGCTTTTCTGGAAGCG
GCTCCGGAACAGATTTCACCCTGAAGATCAGCAGGGTGGAGGCTGAGGAC
CTGGGAGTGTACTTCTGTTCTCAGAGCACCCACGTGCCCCCTCTGACATT
TGGCGCTGGAACCAAGCTGGAGCTGACAGGCCTGCTGGGAGTGTCTCTGG
GAGGAGCTAAGGAGGCTTGCCCAACAGGCCTGTACACCCACAGCGGAGAG
TGCTGTAAGGCCTGTAACCTGGGAGAGGGAGTGGCTCAGCCATGCGGAGC
TAACCAGACCGTGTGCGAGCCCTGTCTGGACTCTGTGACATTCAGCGATG
TGGTGTCCGCTACCGAGCCTTGCAAGCCATGTACAGAGTGCGTGGGCCTG
CAGTCCATGTCTGCCCCATGCGTGGAGGCTGACGATGCCGTGTGCAGGTG
TGCTTACGGCTACTACCAGGACGAGACAACAGGAAGGTGTGAGGCTTGCA
GGGTGTGCGAGGCTGGATCCGGACTGGTGTTTTCTTGCCAGGATAAGCAG
AACACCGTGTGCGAGGAGTGTCCAGACGGCACATACAGCGATGAGGCCAA
CCACGTGGACCCCTGCCTGCCTTGTACCGTGTGCGAGGATACAGAGAGGC
AGCTGAGAGAGTGTACCAGATGGGCTGACGCTGAGTGCGAGGAGATCCCA
GGCCGGTGGATCACACGCTCTACCCCACCAGAGGGAAGCGACTCCACAGC
TCCCAGCACCCAGGAGCCTGAGGCTCCTCCAGAGCAGGATCTGATCGCCT
CTACCGTGGCTGGCGTGGTGACCACAGTGATGGGAAGCTCCCAGCCTGTG
GTGACACGCGGCACCACAGATAACCTGATCCCAGTGTACTGCTCCATCCT
GGCCGCTGTGGTGGTGGGACTCGTGGCATACATTGCTTTCAAACGGTGGA
ATAGGGGAATCCTGTGA
(SEQ ID NO: 18)
MCPQKLTISWFAIVLLVSPLMAMWELEKDVYVVEVDWTPDAPGETVNLTC
DTPEEDDITWTSDQRHGVIGSGKTLTITVKEFLDAGQYTCHKGGETLSHS
HLLLHKKENGIWSTEILKNFKNKTFLKCEAPNYSGRFTCSWLVQRNMDLK
FNIKSSSSSPDSRAVTCGMASLSAEKVTLDQRDYEKYSVSCQEDVTCPTA
EETLPIELALEARQQNKYENYSTSFFIRDIIKPDPPKNLQMKPLKNSQVE
VSWEYPDSWSTPHSYFSLKFFVRIQRKKEKMKETEEGCNQKGAFLVEKTS
TEVQCKGGNVCVQAQDRYYNSSCSKWACVPCRVRSGGGGSGGGGSGGGLA
SGGSMVSVPTASPSASSSSSQCRSSMCQSRYLLFLATLALLNHLSLARVI
PVSGPARCLSQSRNLLKTTDDMVKTAREKLKHYSCTAEDIDHEDITRDQT
STLKTCLPLELHKNESCLATRETSSTTRGSCLPPQKTSLMMTLCLGSIYE
DLKMYQTEFQAINAALQNHNHQQIILDKGMLVAIDELMQSLNHNGETLRQ
KPPVGEADPYRVKMKLCILLHAFSTRVVTINRVMGYLSSATGSGATNFSL
LKQAGDVEENPGPMDFQVQIFSFLLISASVIMSRGEVQLLQSGPELEKPG
ASVMISCKASGSSFTGYNMNWVRQNIGKSLEWIGAIDPYYGGTSYNQKFK
GRATLTVDKSSSTAYMHLKSLTSEDSAVYYCVSGMEYWGQGTSVTVSSGG
GGSGGGGSGGGGSDVVMTQTPLSLPVSLGDQASISCRSSQSLVHRNGNTY
LHWYLQKPGQSPKLLIHKVSNRFSGVPDRFSGSGSGTDFTLKISRVEAED
LGVYFCSQSTHVPPLTFGAGTKLELTGLLGVSLGGAKEACPTGLYTHSGE
CCKACNLGEGVAQPCGANQTVCEPCLDSVTFSDVVSATEPCKPCTECVGL
QSMSAPCVEADDAVCRCAYGYYQDETTGRCEACRVCEAGSGLVFSCQDKQ
NTVCEECPDGTYSDEANHVDPCLPCTVCEDTERQLRECTRWADAECEEIP
GRWITRSTPPEGSDSTAPSTQEPEAPPEQDLIASTVAGVVTTVMGSSQPV
VTRGTTDNLIPVYCSILAAVVVGLVAYIAFKRWNRGIL*
mFLT3L-P2A-GFP
(SEQ ID NO: 19)
ATGGATTTTCAGGTCCAGATTTTCTCCTTCCTCCTCATTTCAGCCAGCGT
CATTATGTCTCGGGGGACACCTGACTGTTACTTCAGCCACAGTCCCATCT
CCTCCAACTTCAAAGTGAAGTTTAGAGAGTTGACTGACCACCTGCTTAAA
GATTACCCAGTCACTGTGGCCGTCAATCTTCAGGACGAGAAGCACTGCAA
GGCCTTGTGGAGCCTCTTCCTAGCCCAGCGCTGGATAGAGCAACTGAAGA
CTGTGGCAGGGTCTAAGATGCAAACGCTTCTGGAGGACGTCAACACCGAG
ATACATTTTGTCACCTCATGTACCTTCCAGCCCCTACCAGAATGTCTGCG
ATTCGTCCAGACCAACATCTCCCACCTCCTGAAGGACACCTGCACACAGC
TGCTTGCTCTGAAGCCCTGTATCGGGAAGGCCTGCCAGAATTTCTCTCGG
TGCCTGGAGGTGCAGTGCCAGCCGGACTCCTCCACCCTGCTGCCCCCAAG
GAGTCCCATAGCCCTAGAAGCCACGGAGCTCCCAGAGCCTCGGCCCAGGC
AGACCGGTTCCGGAGCCACGAACTTCTCTCTGTTAAAGCAAGCAGGAGAC
GTGGAAGAAAACCCCGGTCCTATGGTGAGCAAGGGCGAGGAGCTGTTCAC
CGGGGTGGTGCCCATCCTGGTCGAGCTGGACGGCGACGTAAACGGCCACA
AGTTCAGCGTGTCCGGCGAGGGCGAGGGCGATGCCACCTACGGCAAGCTG
ACCCTGAAGTTCATCTGCACCACCGGCAAGCTGCCCGTGCCCTGGCCCAC
CCTCGTGACCACCCTGACCTACGGCGTGCAGTGCTTCAGCCGCTACCCCG
ACCACATGAAGCAGCACGACTTCTTCAAGTCCGCCATGCCCGAAGGCTAC
GTCCAGGAGCGCACCATCTTCTTCAAGGACGACGGCAACTACAAGACCCG
CGCCGAGGTGAAGTTCGAGGGCGACACCCTGGTGAACCGCATCGAGCTGA
AGGGCATCGACTTCAAGGAGGACGGCAACATCCTGGGGCACAAGCTGGAG
TACAACTACAACAGCCACAACGTCTATATCATGGCCGACAAGCAGAAGAA
CGGCATCAAGGTGAACTTCAAGATCCGCCACAACATCGAGGACGGCAGCG
TGCAGCTCGCCGACCACTACCAGCAGAACACCCCCATCGGCGACGGCCCC
GTGCTGCTGCCCGACAACCACTACCTGAGCACCCAGTCCGCCCTGAGCAA
AGACCCCAACGAGAAGCGCGATCACATGGTCCTGCTGGAGTTCGTGACCG
CCGCCGGGATCACTCTCGGCATGGACGAGCTGTACAAGTAAG
(SEQ ID NO: 20)
MDFQVQIFSFLLISASVIMSRGTPDCYFSHSPISSNFKVKFRELTDHLLK
DYPVTVAVNLQDEKHCKALWSLFLAQRWIEQLKTVAGSKMQTLLEDVNTE
IHFVTSCTFQPLPECLRFVQTNISHLLKDTCTQLLALKPCIGKACQNFSR
CLEVQCQPDSSTLLPPRSPIALEATELPEPRPRQTGSGATNFSLLKQAGD
VEENPGPMVSKGEELFTGVVPILVELDGDVNGHKFSVSGEGEGDATYGKL
TLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGP
VLLPDNHYLSTQSALSKDPNEKRDHMVLLEFVTAAGITLGMDELYK*
hFLT3L-P2A-GFP
(SEQ ID NO: 21)
ATGGATTTTCAGGTCCAGATCTTCTCCTTTCTGCTGATTTCCGCCTCTGT
GATCATGTCCAGAGGCACCCAGGACTGCTCTTTCCAGCACAGCCCTATCA
GCTCCGATTTTGCCGTGAAGATCAGGGAGCTGTCTGACTACCTGCTGCAG
GATTATCCAGTGACCGTGGCCAGCAATCTGCAGGACGAGGAGCTGTGCGG
AGGCCTGTGGAGGCTGGTGCTGGCACAGCGGTGGATGGAGAGACTGAAGA
CAGTGGCCGGCTCTAAGATGCAGGGCCTGCTGGAGCGGGTGAACACCGAG
ATCCACTTCGTGACAAAGTGCGCCTTTCAGCCCCCTCCAAGCTGTCTGCG
GTTTGTGCAGACCAATATCAGCAGACTGCTGCAGGAGACATCCGAGCAGC
TGGTGGCCCTGAAGCCATGGATCACCAGGCAGAACTTCAGCAGGTGCCTG
GAGCTGCAGTGTCAGCCAGACTCTAGCACACTGCCCCCTCCATGGTCCCC
CCGCCCTCTGGAGGCAACCGCACCAACAGCCGGCTCCGGCGCCACCAACT
TTTCTCTGCTGAAGCAGGCCGGCGATGTGGAGGAGAATCCAGGCCCCATG
GTGAGCAAGGGCGAGGAGCTGTTCACAGGCGTGGTGCCCATCCTGGTGGA
GCTGGACGGCGATGTGAATGGCCACAAGTTTAGCGTGTCCGGAGAGGGAG
AGGGCGATGCAACCTACGGCAAGCTGACACTGAAGTTCATCTGCACCACA
GGCAAGCTGCCTGTGCCATGGCCCACCCTGGTGACCACACTGACATACGG
CGTGCAGTGTTTCTCCCGGTATCCCGACCACATGAAGCAGCACGATTTCT
TTAAGTCTGCCATGCCTGAGGGCTACGTGCAGGAGAGGACCATCTTCTTT
AAGGACGATGGCAACTATAAGACCCGCGCCGAGGTGAAGTTCGAGGGCGA
CACACTGGTGAACCGGATCGAGCTGAAGGGCATCGACTTTAAGGAGGATG
GCAATATCCTGGGCCACAAGCTGGAGTACAACTATAATTCCCACAACGTG
TATATCATGGCCGATAAGCAGAAGAACGGCATCAAGGTGAACTTCAAGAT
CAGACACAATATCGAGGACGGCTCTGTGCAGCTGGCCGATCACTACCAGC
AGAACACCCCAATCGGCGACGGACCTGTGCTGCTGCCAGATAATCACTAT
CTGTCTACACAGAGCGCCCTGTCCAAGGACCCTAACGAGAAGAGGGATCA
CATGGTGCTGCTGGAGTTTGTCACTGCCGCTGGGATTACCCTGGGAATGG
ATGAGCTGTATAAATAA
hFLT3L-P2A-GFP
(SEQ ID NO: 22)
MDFQVQIFSFLLISASVIMSRGTQDCSFQHSPISSDFAVKIRELSDYLLQ
DYPVTVASNLQDEELCGGLWRLVLAQRWMERLKTVAGSKMQGLLERVNTE
IHFVTKCAFQPPPSCLRFVQTNISRLLQETSEQLVALKPWITRQNFSRCL
ELQCQPDSSTLPPPWSPRPLEATAPTAGSGATNFSLLKQAGDVEENPGPM
VSKGEELFTGVVPILVELDGDVNGHKFSVSGEGEGDATYGKLTLKFICTT
GKLPVPWPTLVTTLTYGVQCFSRYPDHMKQHDFFKSAMPEGYVQERTIFF
KDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHY
LSTQSALSKDPNEKRDHMVLLEFVTAAGITLGMDELYK*
hIL-12-P2A-dLNGFR
(SEQ ID NO: 23)
ATGTGCCATCAGCAGCTGGTCATCTCTTGGTTTAGTCTGGTGTTTCTGGC
TTCTCCACTGGTCGCAATCTGGGAACTGAAAAAGGACGTGTACGTGGTGG
AGCTGGACTGGTATCCTGATGCCCCAGGCGAGATGGTGGTGCTGACCTGT
GACACACCCGAGGAGGATGGCATCACCTGGACACTGGATCAGAGCTCCGA
GGTGCTGGGAAGCGGCAAGACCCTGACAATCCAGGTGAAGGAGTTCGGCG
ACGCAGGACAGTACACCTGCCACAAGGGAGGCGAGGTGCTGTCTCACAGC
CTGCTGCTGCTGCACAAGAAGGAGGACGGCATCTGGTCCACAGACATCCT
GAAGGATCAGAAGGAGCCCAAGAACAAGACCTTCCTGAGGTGTGAGGCCA
AGAATTATTCTGGCCGCTTTACCTGCTGGTGGCTGACCACAATCTCCACC
GATCTGACATTTTCTGTGAAGTCTAGCAGGGGATCCTCTGACCCTCAGGG
AGTGACATGCGGAGCAGCCACCCTGAGCGCCGAGCGGGTGAGAGGCGATA
ACAAGGAGTACGAGTATTCTGTGGAGTGTCAGGAGGACAGCGCCTGCCCA
GCAGCAGAGGAGTCCCTGCCCATCGAAGTGATGGTGGATGCCGTGCACAA
GCTGAAGTACGAGAATTATACCAGCTCCTTCTTTATCCGGGACATCATCA
AGCCCGATCCCCCTAAGAACCTGCAGCTGAAGCCTCTGAAGAATAGCAGA
CAGGTGGAGGTGTCCTGGGAGTACCCCGACACCTGGTCCACACCTCACAG
CTATTTCTCCCTGACCTTTTGCGTGCAGGTGCAGGGCAAGTCCAAGAGGG
AGAAGAAGGACCGCGTGTTCACCGATAAGACATCTGCCACCGTGATCTGC
CGGAAGAACGCCTCCATCTCTGTGAGGGCCCAGGATCGCTACTATTCTAG
CTCCTGGTCTGAGTGGGCCAGCGTGCCTTGCTCCGGCGGCGGCGGCTCTG
GAGGAGGAGGAAGCGGAGGAGGAGGCTCCAGAAATCTGCCAGTGGCAACC
CCAGACCCTGGAATGTTCCCATGTCTGCACCACTCCCAGAACCTGCTGCG
GGCCGTGTCTAATATGCTGCAGAAGGCCAGACAGACACTGGAGTTTTACC
CTTGCACCAGCGAGGAGATCGACCACGAGGATATCACAAAGGATAAGACC
TCCACAGTGGAGGCCTGTCTGCCACTGGAGCTGACCAAGAACGAGTCTTG
CCTGAATAGCAGGGAGACATCCTTCATCACCAACGGCTCTTGTCTGGCCA
GCCGCAAGACATCCTTTATGATGGCCCTGTGCCTGTCTAGCATCTACGAG
GACCTGAAGATGTATCAGGTGGAGTTCAAGACCATGAACGCCAAGCTGCT
GATGGACCCAAAGCGGCAGATCTTTCTGGATCAGAATATGCTGGCCGTGA
TCGACGAGCTGATGCAGGCCCTGAACTTCAATAGCGAGACAGTGCCTCAG
AAGTCCTCTCTGGAGGAGCCAGATTTCTACAAGACCAAGATCAAGCTGTG
CATCCTGCTGCACGCCTTTAGGATCCGCGCCGTGACAATCGACAGAGTGA
TGTCTTATCTGAACGCAAGCGGATCCGGAGCAACCAACTTCAGCCTGCTG
AAGCAGGCCGGCGATGTGGAGGAGAATCCAGGACCAATGGACGGACCAAG
GTTATTACTGCTGCTGCTGCTGGGCGTGTCTCTGGGAGGAGCAAAGGAGG
CATGCCCAACAGGCCTGTACACCCACAGCGGAGAGTGCTGTAAGGCATGT
AACCTGGGAGAGGGAGTGGCACAGCCTTGCGGAGCCAATCAGACAGTGTG
CGAGCCATGTCTGGACTCTGTGACCTTCAGCGATGTGGTGTCCGCCACAG
AGCCCTGCAAGCCTTGTACCGAGTGCGTGGGCCTGCAGTCTATGAGCGCC
CCTTGCGTGGAGGCAGACGATGCCGTGTGCAGATGTGCCTACGGCTACTA
TCAGGATGAGACAACAGGCCGGTGTGAGGCATGCCGGGTGTGCGAGGCAG
GATCTGGCCTGGTGTTTAGCTGCCAGGACAAGCAGAACACAGTGTGCGAG
GAGTGTCCAGACGGCACCTATTCCGATGAGGCCAATCACGTGGATCCATG
CCTGCCCTGTACAGTGTGCGAGGACACCGAGCGGCAGCTGAGAGAGTGTA
CCAGATGGGCAGATGCAGAGTGCGAGGAGATCCCAGGCCGGTGGATCACA
AGAAGCACCCCACCCGAGGGCTCCGATTCTACAGCACCTTCCACCCAGGA
GCCAGAGGCACCTCCAGAGCAGGACCTGATCGCCAGCACAGTGGCAGGAG
TGGTGACCACAGTGATGGGAAGCTCCCAGCCAGTGGTGACCAGGGGCACC
ACAGACAACCTGATCCCCGTGTACTGCTCCATCCTGGCAGCAGTGGTGGT
GGGCCTGGTGGCCTATATCGCTTTCAAGAGGTGGAATCGGGGCATCCTGT
AA
hIL-12-P2A-dLNGFR
(SEQ ID NO: 24)
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCSGGGGSGGGGSGGGGSRNLPVAT
PDPGMFPCLHHSQNLLRAVSNMLQKARQTLEFYPCTSEEIDHEDITKDKT
STVEACLPLELTKNESCLNSRETSFITNGSCLASRKTSFMMALCLSSIYE
DLKMYQVEFKTMNAKLLMDPKRQIFLDQNMLAVIDELMQALNFNSETVPQ
KSSLEEPDFYKTKIKLCILLHAFRIRAVTIDRVMSYLNASGSGATNFSLL
KQAGDVEENPGPMDGPRLLLLLLLGVSLGGAKEACPTGLYTHSGECCKAC
NLGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVCE
ECPDGTYSDEANHVDPCLPCTVCEDTERQLRECTRWADAECEEIPGRWIT
RSTPPEGSDSTAPSTQEPEAPPEQDLIASTVAGVVTTVMGSSQPVVTRGT
TDNLIPVYCSILAAVVVGLVAYIAFKRWNRGIL*

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1-22. (canceled)

23. An in vitro cell composition that comprises a synthetically partially-differentiated dendritic cell progenitor, wherein the synthetically partially-differentiated dendritic cell progenitor has a phenotype of: CD115⁺, CD11c⁻, and Clec9A⁻, as determined by flow cytometry.

24. The in vitro cell composition of claim 23, wherein the phenotype of the synthetically partially differentiated dendritic progenitor cell further comprises one or more phenotypes selected from CD11b⁻, MHCII⁻, CD45R/B220⁻, and cKIT⁻ as determined by flow cytometry.

25. A differentiated concentional dendritic cell (cDC) comprising cDC1, differentiated from the synthetically partially-differentiated dendritic cell progenitor of claim 23.

26. The differentiated cDC dendritic cell of claim 25, wherein the synthetically-differentiated cDC is an engineered dendritic cell expressing an interleukin or an effector.

27. The differentiated cDC dendritic cell of claim 26, wherein the engineered dendritic cell expresses the interleukin, wherein the interleukin is IL12.

28. The differentiated cDC dendritic cell of claim 26, wherein the engineered dendritic cell expresses the effector, wherein the effector is selected from the group consisting of: extracellular vesicle-internalizing receptor (EVIR), FMS-like tyrosine kinase 3 ligand (FLT3L), IL-12, TNF-α, IL-1, IL-2, IL-6, CXCL8, interferon (IFN), GM-CSF, and G-CSF.

29. An in vitro cell composition that comprises a synthetically partially-differentiated dendritic cell progenitor, wherein the synthetically partially-differentiated dendritic cell progenitor comprises one or more phenotypes selected from CD115⁺, CD34⁺, CD3⁻, CD19⁻, CD335⁻, CD66b⁻, CD10⁻ and CD14⁻ as determined by flow cytometry.

30. An antigen-presenting cell (APC) differentiated from the synthetically partially-differentiated dendritic cell progenitor of claim 29.

31. The APC of claim 30, wherein the APC is an engineered dendritic cell expressing an interleukin or an effector.

32. The APC of claim 31, wherein the engineered dendritic cell expresses the interleukin, wherein the interleukin is IL12.

33. The APC of claim 31, wherein the engineered dendritic cell expresses the effector, wherein the effector is selected from the group consisting of: extracellular vesicle-internalizing receptor (EVIR), FMS-like tyrosine kinase 3 ligand (FLT3L), GM-CSF, IL-6, IL-12, IFNα2β, IFNγ, SCF, and TNF-α.

34. A method of making a synthetically partially differentiated dendritic cell progenitor, the method comprising:
(a) obtaining a shortly-expanded hematopoietic stem/progenitor cell (HSPC); and
(b) contacting the shortly-expanded HSPC with a synthetic medium comprising FMS-like tyrosine kinase 3 ligand (FLT3L) and GM-CSF, with or without IL-1, IL-2, IL-4, IL-6, IL-12, CXCL8, G-CSF, TNF-α, IFNa, PGE2, or retronectin, in an amount sufficient to differentiate the HSPC cell into a synthetically partially-differentiated dendritic cell progenitor having a phenotype of: CD115⁺, CD11c⁻, and Clec9A⁻, as determined by flow cytometry.

35. The method of claim 34, further comprising contacting the HSPC in a medium comprising: FBS, L-glutamine, SCF, TPO, FLT3L, IL-3, IL-6, and IL-1b, thereby making the shortly-expanded HSPC prior to the contacting of (b).

36. A method of making a synthetically partially differentiated dendritic cell progenitor, the method comprising:
(a) obtaining a shortly-expanded CD34⁺ human hematopoietic stem progenitor cell (human HSPC); and
(b) contacting the shortly-expanded human HSPC with a synthetic medium comprising FMS-like tyrosine kinase 3 ligand (FLT3L), IL-3, IL-6, TPO, and SCF, with or without IFNγ, IL-12, retronectin, TNF-α, or UM729, in an amount sufficient to differentiate the HSPC cell into a synthetically partially-differentiated dendritic cell progenitor having one or more phenotypes selected from CD115⁺, CD34⁺, CD3⁻, CD19⁻, CD335⁻, CD66b⁻, CD10⁻, and CD14⁻ as determined by flow cytometry.

37. A pharmaceutical composition for use in treatment of a condition, comprising: (a) the in vitro cell composition of claim 23, and (b) a pharmaceutically-acceptable excipient, diluent, or carrier.

38. The pharmaceutical composition of claim 37, wherein the condition is a cancer.

39. The pharmaceutical composition of claim 37, further comprising an interleukin or an effector.

40. The pharmaceutical composition of claim 37, wherein the pharmaceutical composition comprises the differentiated cDC dendritic cell, and wherein the differentiated cDC dendritic cell is an engineered dendritic cell that expresses an interleukin or an effector.

41. The pharmaceutical composition of claim 37, wherein the pharmaceutical composition comprises the APC, and wherein the APC is an engineered dendritic cell that expresses an interleukin or an effector.

42. The pharmaceutical composition of claim 39, wherein the interleukin is IL-12.

43. The pharmaceutical composition of claim 39, wherein the effector is selected from the group consisting of: extracellular vesicle-internalizing receptor (EVIR), FMS-like tyrosine kinase 3 ligand (FLT3L), IL-12, TNF-α, IL-1, IL-2, IL-6, CXCL8, interferon (IFN), GM-CSF, and G-CSF.

44. The pharmaceutical composition of claim 39, wherein the effector is not expressed on a cell of the cancer.
